SEQUENCE LISTING (170 sequences)

SEQ ID NO: 1 - protein, 12 aa, synthetic construct
  SITE 1: note = Acetyl tyrosine
  SITE 12: note = valine amide
YSMEHFRWGKPV

SEQ ID NO: 2 - protein, 11 aa, synthetic construct
  SITE 1: note = Acetyl serine
  SITE 11: note = valine amide
SMEHFRWGKPV

SEQ ID NO: 3 - protein, 10 aa, synthetic construct
  SITE 1: note = Acetyl methionine
  SITE 10: note = valine amide
MEHFRWGKPV

SEQ ID NO: 4 - protein, 9 aa, synthetic construct
  SITE 1: note = Acetyl glutamic acid
  SITE 9: note = valine amide
EHFRWGKPV

SEQ ID NO: 5 - protein, 12 aa, synthetic construct
  SITE 1: note = Acetyl serine
  SITE 12: note = proline amide
SYSMEHFRWGKP

SEQ ID NO: 6 - protein, 11 aa, synthetic construct
  SITE 1: note = Acetyl serine
  SITE 11: note = lysine amide
SYSMEHFRWGK

SEQ ID NO: 7 - protein, 8 aa, synthetic construct
  SITE 1: note = Acetyl glutamic acid
  SITE 8: note = proline amide
EHFRWGKP

SEQ ID NO: 8 - protein, 7 aa, synthetic construct
  SITE 1: note = Acetyl norleucine
  SITE 2: note = (s)-2-amino-6-azidohexanoic acid
  SITE 7: note = propargyl-glycineamide
  SITE 2..7: note = triazole bridge
XKHFRWX

SEQ ID NO: 9 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 7: note = propargyl-glycineamide
  SITE 2..7: note = triazole bridge
XKHFRWX

SEQ ID NO: 10 - protein, 8 aa, synthetic construct
  SITE 1: note = N2-acetyl-N6-diazo-L-lysine
  SITE 8: note = propargyl-glycineamide
  SITE 1..8: note = triazole bridge
KEHFRWGX

SEQ ID NO: 11 - protein, 6 aa, synthetic construct
  SITE 1: note = acetyl glutamic acid
  SITE 6: note = cysteinamide
  SITE 1..6: note = thioether bridge
EHFRWC

SEQ ID NO: 12 - protein, 7 aa, synthetic construct
  SITE 1: note = acetyl glutamic acid
  SITE 7: note = cysteinamide
  SITE 1..7: note = thioether bridge
EHFRWGC

SEQ ID NO: 13 - protein, 8 aa, synthetic construct
  SITE 1: note = Acetyl R-2-(4-pentenyl)alanine
  SITE 8: note = S-2-(4-pentenyl) alanine amide
  SITE 1..8: note = hydrocarbon staple
XEHFRWGX

SEQ ID NO: 14 - protein, 8 aa, synthetic construct
  SITE 1: note = Acetyl S-2-(4-pentenyl) alanine
  SITE 8: note = S-2-(4-pentenyl) alanine amide
  SITE 1..8: note = hydrocarbon staple
XEHFRWGX

SEQ ID NO: 15 - protein, 8 aa, synthetic construct
  SITE 1: note = Acetyl R-2-(7-octenyl) alanine
  SITE 8: note = S-2-(4-pentenyl) alanine amide
  SITE 1..8: note = hydrocarbon staple
XEHFRWGX

SEQ ID NO: 16 - protein, 13 aa, synthetic construct
  SITE 1: note = 4-phenyl-butanoic acid serine
  SITE 13: note = valine amide
SYSMEHFRWGKPV

SEQ ID NO: 17 - protein, 15 aa, synthetic construct
  SITE 1: note = Octanoyl polyethylene glycol-8 glycine
  SITE 15: note = valine amide
GGSYSMEHFRWGKPV

SEQ ID NO: 18 - protein, 13 aa, synthetic construct
  SITE 1: note = 4-pyridylacetyl acid serine
  SITE 13: note = valine amide
SYSMEHFRWGKPV

SEQ ID NO: 19 - protein, 13 aa, synthetic construct
  SITE 1: note = 3,5-hydroxybenzoic acid serine
  SITE 13: note = valine amide
SYSMEHFRWGKPV

SEQ ID NO: 20 - protein, 13 aa, synthetic construct
  SITE 1: note = Octanoyl serine
  SITE 13: note = valine amide
SYSMEHFRWGKPV

SEQ ID NO: 21 - protein, 13 aa, synthetic construct
  SITE 1: note = Acetyl serine
  SITE 6: note = N-methyl histidine
  SITE 13: note = valine amide
SYSMEHFRWGKPV

SEQ ID NO: 22 - protein, 13 aa, synthetic construct
  SITE 1: note = Acetyl serine
  SITE 7: note = N-methyl phenylalanine
  SITE 13: note = valine amide
SYSMEHFRWGKPV

SEQ ID NO: 23 - protein, 13 aa, synthetic construct
  SITE 1: note = Acetyl serine
  SITE 8: note = N-methyl arginine
  SITE 13: note = valine amide
SYSMEHFRWGKPV

SEQ ID NO: 24 - protein, 13 aa, synthetic construct
  SITE 1: note = Acetyl serine
  SITE 9: note = N-methyl tryptophan
  SITE 13: note = valine amide
SYSMEHFRWGKPV

SEQ ID NO: 25 - protein, 13 aa, synthetic construct
  SITE 1: note = Acetyl serine
  SITE 7: note = N-methyl-D-phenylalanine
  SITE 13: note = valine amide
SYSMEHFRWGKPV

SEQ ID NO: 26 - protein, 7 aa, synthetic construct
  SITE 1: note = Acetyl norleucine
  SITE 2: note = R-2-(4-pentenyl)alanine
  SITE 4: note = D-phenylalanine
  SITE 6: note = S-2-(4-pentenyl) alanine
  SITE 7: note = tryptophan amide
XXHFRXW

SEQ ID NO: 27 - protein, 7 aa, synthetic construct
  SITE 1: note = Acetyl norleucine
  SITE 2: note = N-(2-carboxyethyl)glycine
  SITE 4: note = D-phenylalanine
  SITE 6: note = Diaminobuyric acid
  SITE 7: note = tryptophan amide
XXHFRXW

SEQ ID NO: 28 - protein, 7 aa, synthetic construct
  SITE 1: note = Acetyl norleucine
  SITE 4: note = D-phenylalanine
  SITE 6: note = N-(2-aminoethyl) glycine
  SITE 7: note = tryptophan amide
XEHFRXW

SEQ ID NO: 29 - protein, 7 aa, synthetic construct
  SITE 1: note = Acetyl norleucine
  SITE 2: note = N-(2-carboxyethyl)glycine
  SITE 4: note = D-phenylalanine
  SITE 6: note = N-(2-aminoethyl) glycine
  SITE 7: note = tryptophan amide
XXHFRXW

SEQ ID NO: 30 - protein, 7 aa, synthetic construct
  SITE 1: note = Acetyl norleucine
  SITE 2: note = N-(2-aminoethyl) glycine
  SITE 4: note = D-phenylalanine
  SITE 6: note = N-(2-aminoethyl) glycine
XXHFRXW

SEQ ID NO: 31 - protein, 7 aa, synthetic construct
  SITE 1: note = Acetyl norleucine
  SITE 2: note = alpha methyl cysteine
  SITE 4: note = D-phenylalanine
  SITE 6: note = alpha methyl cysteine
  SITE 7: note = tryptophan amide
XCHFRCW

SEQ ID NO: 32 - protein, 7 aa, synthetic construct
  SITE 1: note = Acetyl norleucine
  SITE 4: note = D-phenylalanine
XCHFRCW

SEQ ID NO: 33 - protein, 8 aa, synthetic construct
  SITE 1: note = Acetyl R-2-(7-octenyl) alanine
  SITE 4: note = D-phenylalanine
  SITE 8: note = S-2-(7-octenyl) alanine amide
  SITE 1..8: note = hydrocarbon staple
XEHFRWGX

SEQ ID NO: 34 - protein, 8 aa, synthetic construct
  SITE 1: note = Acetyl norleucine
  SITE 2: note = R-2-(7-octenyl) alanine
  SITE 4: note = D-phenylalanine
  SITE 7: note = S-2-(4-pentenyl) alanine
  SITE 8: note = glycineamide
  SITE 2..7: note = hydrocarbon staple
XXHFRWXG

SEQ ID NO: 35 - protein, 8 aa, synthetic construct
  SITE 1: note = Acetyl norleucine
  SITE 8: note = lysine amide
XEHFRWGK

SEQ ID NO: 36 - protein, 9 aa, synthetic construct
  SITE 1: note = acetyl norleucine
  SITE 9: note = cysteinamide
XEHFRWGKC

SEQ ID NO: 37 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 38 - protein, 7 aa, synthetic construct
  SITE 1: note = Acetyl norleucine
  SITE 2: note = R-2-(4-pentenyl)alanine
  SITE 4: note = D-phenylalanine
  SITE 7: note = S-2-(4-pentenyl) alanine amide
XXHFRWX

SEQ ID NO: 39 - protein, 7 aa, synthetic construct
  SITE 1: note = Acetyl norleucine
  SITE 2: note = S-2-(4-pentenyl) alanine
  SITE 4: note = D-phenylalanine
  SITE 7: note = S-2-(4-pentenyl) alanine amide
XXHFRWX

SEQ ID NO: 40 - protein, 7 aa, synthetic construct
  SITE 1: note = Acetyl norleucine
  SITE 4: note = D-phenylalanine
XCHFRWC

SEQ ID NO: 41 - protein, 7 aa, synthetic construct
  SITE 1: note = Acetyl norleucine
  SITE 2: note = Diaminobuyric acid
  SITE 4: note = D-phenylalanine
  SITE 7: note = Diaminobuyric acid
XXHFRWX

SEQ ID NO: 42 - protein, 7 aa, synthetic construct
  SITE 1: note = Acetyl norleucine
  SITE 2: note = (S)-2-Amino-4-azidobutanoic acid
  SITE 4: note = D-phenylalanine
  SITE 7: note = (S)-2-Amino-4-azidobutanoic acid
XXHFRWX

SEQ ID NO: 43 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 7: note = propargyl-glycineamide
XKHFRWX

SEQ ID NO: 44 - protein, 7 aa, synthetic construct
  SITE 1: note = Acetyl norleucine
  SITE 2: note = (s)-2-amino-6-azidohexanoic acid
  SITE 4: note = N-methyl D-phenylalanine
  SITE 7: note = propargyl-glycineamide
XKHFRWX

SEQ ID NO: 45 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 5: note = N-methyl arginine
  SITE 7: note = propargyl-glycineamide
XKHFRWX

SEQ ID NO: 46 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 6: note = N-methyl tryptophan
  SITE 7: note = propargyl-glycineamide
XKHFRWX

SEQ ID NO: 47 - protein, 7 aa, synthetic construct
  SITE 1: note = Acetyl norleucine
  SITE 2: note = (s)-2-amino-6-azidohexanoic acid
  SITE 3: note = 4-amino-phenylalanine
  SITE 4: note = D-phenylalanine
  SITE 7: note = propargyl-glycineamide
XKFFRWX

SEQ ID NO: 48 - protein, 7 aa, synthetic construct
  SITE 1: note = Acetyl norleucine
  SITE 2: note = (s)-2-amino-6-azidohexanoic acid
  SITE 4: note = 4-trifluoromethyl-phenylalanine
  SITE 7: note = propargyl-glycineamide
XKHFRWX

SEQ ID NO: 49 - protein, 7 aa, synthetic construct
  SITE 1: note = Acetyl norleucine
  SITE 2: note = (s)-2-amino-6-azidohexanoic acid
  SITE 4: note = 4-fluoro-D-phenylalanine
  SITE 7: note = propargyl-glycineamide
XKHFRWX

SEQ ID NO: 50 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 5: note = N-methyl arginine
  SITE 6: note = N-methyl tryptophan
  SITE 7: note = propargyl-glycineamide
XKHFRWX

SEQ ID NO: 51 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 6: note = N-methyl tryptophan
  SITE 7: note = propargyl-glycineamide
XKHFRWX

SEQ ID NO: 52 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 5: note = N-methyl arginine
  SITE 7: note = propargyl-glycineamide
XKHFRWX

SEQ ID NO: 53 - protein, 8 aa, synthetic construct
  SITE 1: note = 3,5-hydroxybenzoic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 54 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 5: note = Homoarginine
  SITE 7: note = propargylglycine
  SITE 8: note = lysine amide
XKHFRWXK

SEQ ID NO: 55 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 5: note = N-omega,N'-omega-dimethyl-L-arginine
  SITE 7: note = propargylglycine
  SITE 8: note = lysine amide
XKHFRWXK

SEQ ID NO: 56 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = asparagine amide
XKHFRWXN

SEQ ID NO: 57 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = homoglutaminamide
XKHFRWXQ

SEQ ID NO: 58 - protein, 8 aa, synthetic construct
  SITE 1: note = Acetyl norleucine
  SITE 2: note = (s)-2-amino-6-azidohexanoic acid
  SITE 4: note = O-methyl-D-tyrosine
  SITE 7: note = propargylglycine
  SITE 8: note = lysine amide
XKHYRWXK

SEQ ID NO: 59 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = N-omega-dimethyl-lysine amide
XKHFRWXK

SEQ ID NO: 60 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 6: note = 1-methyl-tryptophan
  SITE 7: note = propargylglycine
  SITE 8: note = lysine amide
XKHFRWXK

SEQ ID NO: 61 - protein, 8 aa, synthetic construct
  SITE 1: note = Acetyl norleucine
  SITE 2: note = (s)-2-amino-6-azidohexanoic acid
  SITE 3: note = O-methyl-tyrosine
  SITE 4: note = D-phenylalanine
  SITE 7: note = propargylglycine
  SITE 8: note = lysine amide
XKXFRWXK

SEQ ID NO: 62 - protein, 8 aa, synthetic construct
  SITE 1: note = 3,5-dihydroxyphenylacetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 63 - protein, 8 aa, synthetic construct
  SITE 1: note = (3-methoxyphenyl)acetic acid (3-methoxyphenyl)acetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 64 - protein, 8 aa, synthetic construct
  SITE 1: note = 3-hydroxy-5-methoxybenzeneacetic acid 3-hydroxy-5-methoxybenzeneacetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 65 - protein, 8 aa, synthetic construct
  SITE 1: note = Acetyl norleucine
  SITE 2: note = (S)-2-amino-5-azidopentanoic acid
  SITE 4: note = D-phenylalanine
  SITE 7: note = propargylglycine
  SITE 8: note = lysine amide
XXHFRWXK

SEQ ID NO: 66 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = homopropargylglycine
  SITE 8: note = lysine amide
XKHFRWXK

SEQ ID NO: 67 - protein, 8 aa, synthetic construct
  SITE 1: note = Acetyl norleucine
  SITE 2: note = propargylglycine
  SITE 4: note = D-phenylalanine
  SITE 7: note = (s)-2-amino-6-azidohexanoic acid
  SITE 8: note = lysine amide
XXHFRWKK

SEQ ID NO: 68 - protein, 8 aa, synthetic construct
  SITE 1: note = Acetyl norleucine
  SITE 2: note = (S)-2-amino-5-azidopentanoic acid
  SITE 4: note = D-phenylalanine
  SITE 7: note = homopropargylglycine
  SITE 8: note = lysine amide
XXHFRWXK

SEQ ID NO: 69 - protein, 8 aa, synthetic construct
  SITE 1: note = Acetyl norleucine
  SITE 2: note = (R)-2-amino-6-azidohexanoic acid
  SITE 4: note = D-phenylalanine
  SITE 7: note = propargylglycine
  SITE 8: note = lysine amide
XKHFRWXK

SEQ ID NO: 70 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = D-propargylglycine
  SITE 8: note = lysine amide
XKHFRWXK

SEQ ID NO: 71 - protein, 8 aa, synthetic construct
  SITE 1: note = 3,5-hydroxybenzoic acid norleucine
  SITE 2: note = (s)-2-amino-6-azidohexanoic acid
  SITE 4: note = 4-fluoro-D-phenylalanine
  SITE 7: note = propargylglycine
  SITE 8: note = homoglutaminamide
XKHFRWXQ

SEQ ID NO: 72 - protein, 7 aa, synthetic construct
  SITE 1: note = 3,5-hydroxybenzoic acid norleucine
  SITE 2: note = (s)-2-amino-6-azidohexanoic acid
  SITE 4: note = 4-fluoro-D-phenylalanine
  SITE 7: note = propargyl-glycineamide
XKHFRWX

SEQ ID NO: 73 - protein, 7 aa, synthetic construct
  SITE 1: note = 3,5-hydroxybenzoic acid norleucine
  SITE 2: note = (s)-2-amino-6-azidohexanoic acid
  SITE 4: note = D-phenylalanine
  SITE 7: note = propargyl-glycineamide
XKHFRWX

SEQ ID NO: 74 - protein, 7 aa, synthetic construct
  SITE 1: note = 4-pyridylacetyl acid norleucine
  SITE 2: note = (s)-2-amino-6-azidohexanoic acid
  SITE 4: note = D-phenylalanine
  SITE 7: note = propargyl-glycineamide
XKHFRWX

SEQ ID NO: 75 - protein, 7 aa, synthetic construct
  SITE 1: note = 3,5-hydroxybenzoic acid norleucine
  SITE 2: note = (s)-2-amino-6-azidohexanoic acid
  SITE 3: note = N-methyl histidine
  SITE 4: note = 4-fluoro-D-phenylalanine
  SITE 6: note = N-methyl tryptophan
  SITE 7: note = propargyl-glycineamide
XKHFRWX

SEQ ID NO: 76 - protein, 13 aa, synthetic construct
  SITE 1: note = Acetyl serine
  SITE 13: note = valine amide
SYSMEHFRWGKPV

SEQ ID NO: 77 - protein, 13 aa, synthetic construct
  SITE 1: note = Acetyl serine
  SITE 4: note = Norleucine
  SITE 7: note = D-phenylalanine
  SITE 13: note = valine amide
SYSXEHFRWGKPV

SEQ ID NO: 78 - protein, 39 aa, synthetic construct
  SITE 1: note = Acetyl serine
  SITE 39: note = phenylalanine amide
SYSMEHFRWGKPVGKKRRPVKVYPNGAEDESAEAFPLEF

SEQ ID NO: 79 - protein, 7 aa, synthetic construct
  SITE 1: note = Acetyl norleucine
  SITE 4: note = D-phenylalanine
  SITE 6: note = Diaminobuyric acid
  SITE 7: note = tryptophan amide
XEHFRXW

SEQ ID NO: 80 - protein, 266 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 81 - protein, 7 aa, synthetic construct
MEHFRWG

SEQ ID NO: 82 - protein, 4 aa, synthetic construct
HFRW

SEQ ID NO: 83 - protein, 10 aa, synthetic construct
  SITE 1: note = Acetyl serine
  SITE 10: note = glycineamide
SYSMEHFRWG

SEQ ID NO: 84 - protein, 9 aa, synthetic construct
  SITE 1: note = Acetyl serine
  SITE 9: note = tryptophan amide
SYSMEHFRW

SEQ ID NO: 85 - protein, 11 aa, synthetic construct
  SITE 1: note = Acetyl tyrosine
  SITE 11: note = proline amide
YSMEHFRWGKP

SEQ ID NO: 86 - protein, 10 aa, synthetic construct
  SITE 1: note = Acetyl tyrosine
  SITE 10: note = lysine amide
YSMEHFRWGK

SEQ ID NO: 87 - protein, 9 aa, synthetic construct
  SITE 1: note = Acetyl tyrosine
  SITE 9: note = glycineamide
YSMEHFRWG

SEQ ID NO: 88 - protein, 8 aa, synthetic construct
  SITE 1: note = Acetyl tyrosine
  SITE 8: note = tryptophan amide
YSMEHFRW

SEQ ID NO: 89 - protein, 10 aa, synthetic construct
  SITE 1: note = Acetyl serine
  SITE 10: note = proline amide
SMEHFRWGKP

SEQ ID NO: 90 - protein, 9 aa, synthetic construct
  SITE 1: note = Acetyl serine
  SITE 9: note = lysine amide
SMEHFRWGK

SEQ ID NO: 91 - protein, 8 aa, synthetic construct
  SITE 1: note = Acetyl serine
  SITE 8: note = glycineamide
SMEHFRWG

SEQ ID NO: 92 - protein, 7 aa, synthetic construct
  SITE 1: note = Acetyl serine
  SITE 7: note = tryptophan amide
SMEHFRW

SEQ ID NO: 93 - protein, 9 aa, synthetic construct
  SITE 1: note = Acetyl methionine
  SITE 9: note = proline amide
MEHFRWGKP

SEQ ID NO: 94 - protein, 8 aa, synthetic construct
  SITE 1: note = Acetyl methionine
  SITE 8: note = lysine amide
MEHFRWGK

SEQ ID NO: 95 - protein, 7 aa, synthetic construct
  SITE 1: note = Acetyl methionine
  SITE 7: note = glycineamide
MEHFRWG

SEQ ID NO: 96 - protein, 6 aa, synthetic construct
  SITE 1: note = Acetyl methionine
  SITE 6: note = tryptophan amide
MEHFRW

SEQ ID NO: 97 - protein, 7 aa, synthetic construct
  SITE 1: note = Acetyl glutamic acid
  SITE 7: note = lysine amide
EHFRWGK

SEQ ID NO: 98 - protein, 6 aa, synthetic construct
  SITE 1: note = Acetyl glutamic acid
  SITE 6: note = glycineamide
EHFRWG

SEQ ID NO: 99 - protein, 5 aa, synthetic construct
  SITE 1: note = Acetyl glutamic acid
  SITE 5: note = tryptophan amide
EHFRW

SEQ ID NO: 100 - protein, 8 aa, synthetic construct
  SITE 1: note = Acetyl histidine
  SITE 8: note = valine amide
HFRWGKPV

SEQ ID NO: 101 - protein, 7 aa, synthetic construct
  SITE 1: note = Acetyl histidine
  SITE 7: note = proline amide
HFRWGKP

SEQ ID NO: 102 - protein, 6 aa, synthetic construct
  SITE 1: note = Acetyl histidine
  SITE 6: note = lysine amide
HFRWGK

SEQ ID NO: 103 - protein, 5 aa, synthetic construct
  SITE 1: note = Acetyl histidine
  SITE 5: note = glycineamide
HFRWG

SEQ ID NO: 104 - protein, 4 aa, synthetic construct
  SITE 1: note = Acetyl histidine
  SITE 4: note = tryptophan amide
HFRW

SEQ ID NO: 105 - protein, 6 aa, synthetic construct
LLLLLL

SEQ ID NO: 106 - protein, 6 aa, synthetic construct
GLLLLL

SEQ ID NO: 107 - protein, 6 aa, synthetic construct
LGLLLL

SEQ ID NO: 108 - protein, 6 aa, synthetic construct
LLGLLL

SEQ ID NO: 109 - protein, 6 aa, synthetic construct
LLLGLL

SEQ ID NO: 110 - protein, 6 aa, synthetic construct
LLLLGL

SEQ ID NO: 111 - protein, 6 aa, synthetic construct
LLLLLG

SEQ ID NO: 112 - protein, 6 aa, synthetic construct
GGLLLL

SEQ ID NO: 113 - protein, 6 aa, synthetic construct
GLGLLL

SEQ ID NO: 114 - protein, 6 aa, synthetic construct
GLLGLL

SEQ ID NO: 115 - protein, 6 aa, synthetic construct
GLLLGL

SEQ ID NO: 116 - protein, 6 aa, synthetic construct
GLLLLG

SEQ ID NO: 117 - protein, 6 aa, synthetic construct
LGGLLL

SEQ ID NO: 118 - protein, 6 aa, synthetic construct
LGLGLL

SEQ ID NO: 119 - protein, 6 aa, synthetic construct
LGLLGL

SEQ ID NO: 120 - protein, 6 aa, synthetic construct
LGLLLG

SEQ ID NO: 121 - protein, 6 aa, synthetic construct
LLGGLL

SEQ ID NO: 122 - protein, 6 aa, synthetic construct
LLGLGL

SEQ ID NO: 123 - protein, 6 aa, synthetic construct
LLGLLG

SEQ ID NO: 124 - protein, 6 aa, synthetic construct
LLLGGL

SEQ ID NO: 125 - protein, 6 aa, synthetic construct
LLLGLG

SEQ ID NO: 126 - protein, 6 aa, synthetic construct
LLLLGG

SEQ ID NO: 127 - protein, 6 aa, synthetic construct
GGGLLL

SEQ ID NO: 128 - protein, 6 aa, synthetic construct
GGLGLL

SEQ ID NO: 129 - protein, 6 aa, synthetic construct
GGLLGL

SEQ ID NO: 130 - protein, 6 aa, synthetic construct
GGLLLG

SEQ ID NO: 131 - protein, 6 aa, synthetic construct
GLGGLL

SEQ ID NO: 132 - protein, 6 aa, synthetic construct
GLGLGL

SEQ ID NO: 133 - protein, 6 aa, synthetic construct
GLGLLG

SEQ ID NO: 134 - protein, 6 aa, synthetic construct
GLLGGL

SEQ ID NO: 135 - protein, 6 aa, synthetic construct
GLLGLG

SEQ ID NO: 136 - protein, 6 aa, synthetic construct
GLLLGG

SEQ ID NO: 137 - protein, 6 aa, synthetic construct
LLLGGG

SEQ ID NO: 138 - protein, 6 aa, synthetic construct
LLGLGG

SEQ ID NO: 139 - protein, 6 aa, synthetic construct
LLGGLG

SEQ ID NO: 140 - protein, 6 aa, synthetic construct
LLGGGL

SEQ ID NO: 141 - protein, 6 aa, synthetic construct
LGLLGG

SEQ ID NO: 142 - protein, 6 aa, synthetic construct
LGLGLG

SEQ ID NO: 143 - protein, 6 aa, synthetic construct
LGLGGL

SEQ ID NO: 144 - protein, 6 aa, synthetic construct
LGGLLG

SEQ ID NO: 145 - protein, 6 aa, synthetic construct
LGGLGL

SEQ ID NO: 146 - protein, 6 aa, synthetic construct
LGGGLL

SEQ ID NO: 147 - protein, 6 aa, synthetic construct
LLGGGG

SEQ ID NO: 148 - protein, 6 aa, synthetic construct
LGLGGG

SEQ ID NO: 149 - protein, 6 aa, synthetic construct
LGGLGG

SEQ ID NO: 150 - protein, 6 aa, synthetic construct
LGGGLG

SEQ ID NO: 151 - protein, 6 aa, synthetic construct
LGGGGL

SEQ ID NO: 152 - protein, 6 aa, synthetic construct
GLLGGG

SEQ ID NO: 153 - protein, 6 aa, synthetic construct
GLGLGG

SEQ ID NO: 154 - protein, 6 aa, synthetic construct
GLGGLG

SEQ ID NO: 155 - protein, 6 aa, synthetic construct
GLGGGL

SEQ ID NO: 156 - protein, 6 aa, synthetic construct
GGLLGG

SEQ ID NO: 157 - protein, 6 aa, synthetic construct
GGLGLG

SEQ ID NO: 158 - protein, 6 aa, synthetic construct
GGLGGL

SEQ ID NO: 159 - protein, 6 aa, synthetic construct
GGGLLG

SEQ ID NO: 160 - protein, 6 aa, synthetic construct
GGGLGL

SEQ ID NO: 161 - protein, 6 aa, synthetic construct
GGGGLL

SEQ ID NO: 162 - protein, 6 aa, synthetic construct
LGGGGG

SEQ ID NO: 163 - protein, 6 aa, synthetic construct
GLGGGG

SEQ ID NO: 164 - protein, 6 aa, synthetic construct
GGLGGG

SEQ ID NO: 165 - protein, 6 aa, synthetic construct
GGGLGG

SEQ ID NO: 166 - protein, 6 aa, synthetic construct
GGGGLG

SEQ ID NO: 167 - protein, 6 aa, synthetic construct
GGGGGL

SEQ ID NO: 168 - protein, 6 aa, synthetic construct
GGGGGG

SEQ ID NO: 169 - protein, 4 aa, synthetic construct
  SITE 1: note = palmitic acid polyethylene glycol-8 glycine
GGSY

SEQ ID NO: 170 - protein, 5 aa, synthetic construct
  SITE 1: note = Acetyl palmitoyl lysine
KGGSY

(19) United States
(12) Patent Application Publication
(10) Pub. No.: US 2026/0152527 A1
(43) Pub. Date: Jun. 4, 2026

(54) NOVEL MELANOCORTIN ANALOGS

(71) Applicant: SightStream Biotherapeutics, Inc., Dallas, TX (US)

(72) Inventors: Norris Andrew Whitlock, Mount Pleasant, SC (US); Jason M. Werner, Dallas, TX (US); Yvonne Angell, Shanghai (CN); Yan Wang, San Francisco, CA (US); Renato Skerlj, Newton, MA (US)

(21) Appl. No.: 19/100,381

(22) PCT Filed: Aug. 2, 2023
(86) PCT No.: PCT/US2023/071547

§ 371 Date: Jan. 31, 2025

Related U.S. Application Data
(60) Provisional application No. 63/394,868, filed on Aug. 3, 2022.

(51) Int. Cl. C07K 7/08; A61K 38/00; A61P 27/02

(52) U.S. Cl. CPC C07K 7/08; A61P 27/02; A61K 38/00

(57) ABSTRACT

The present disclosure presents melanocortin receptor-specific peptides (e.g., novel α-MSH peptide analogs) which may be used in the treatment of melanocortin receptor-mediated diseases, indications, conditions, and syndromes.

### CROSS-REFERENCE TO RELATED APPLICATION(S)

This application claims priority to, and the benefit of, co-pending U.S. Provisional Application No. 63/394,868, filed Aug. 3, 2022. The disclosures of said provisional application are hereby incorporated by reference in their entirety.

### STATEMENT REGARDING SEQUENCE LISTING

The Sequence Listing associated with this application is provided in .xml format in lieu of a paper copy and is hereby incorporated by reference into the specification. The name of the .xml file containing the Sequence Listing is KERA-005-WO1_Seq_Listing.xml. The xml file is 275 kb, was created on Jul. 7, 2022, and is being submitted electronically via Patent Center.

### FIELD OF THE DISCLOSURE

The present disclosure presents melanocortin receptor-specific peptides (e.g., novel α-MSH peptide analogs) which may be used in the treatment of melanocortin receptor-mediated diseases, indications, conditions, and syndromes.

### BACKGROUND

Eye is an organ comprising a number of components, including the cornea, aqueous humor, lens, vitreous humor, retina, the retinal pigment epithelium, and choroid. Ocular diseases are conditions affecting the different tissues of the eye. A number of diseases and disorders affect the different components of the eye, and may cause impaired vision, full or partial blindness, irritation, dryness, sensitivity, photophobia, and/or light aversion.

Melanocortin receptors (MCRs) are ubiquitous in the eye. Human endothelial cells were recently found to express certain melanocortin receptors (MCRs), including the MC1-receptor (MC1-R). These ocular MC1-receptors thus have great potential for being targeted in the treatment of various ophthalmic indications, including ophthalmic diseases related to corneal endothelial cells (CEnC). However, many melanocortins (and analogs thereof) have limited selectivity between various MCRs and tissue types, with few MCR-specific peptides having been identified as effective therapeutics or approved for pharmaceutical applications.

A strong need therefore exists for novel MCR-targeting compounds (e.g., novel α-MSH peptide analogs) which are highly specific to particular MCR subtypes, including ophthalmic tissues, retina, corneal epithelium and corneal MCRs related to ophthalmic diseases.

### SUMMARY

Novel MCR-targeting compounds (e.g., novel α-MSH peptide analogs) which are highly specific to particular MCR subtypes are described herein. The α-MSH peptide analogs may be used for the treatment of one or more ophthalmic indications in a subject.

Disclosed herein are peptides of the formula: Z-XAA1-XAA2-XAA3-XAA4-XAA5-XAA6-XAA7-XAA8-XAA9-XAA10-XAA11-XAA12-XAA13-Y, wherein: Z is Ac, PBA, octanoyl-PEG8G-G, PyAA, HyBA, HyPA, MoPA, HymBA, C7, C8, or C9; AA1, AA2, AA3 are absent; AA4 is Met or Nle or Lys(N3) or optionally Lys(N3) forms a cyclic peptide as a triazole group with Pra group at the 11 position; AA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F), Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 10 position; AA11 is absent, Pra or optionally Pra forms a cyclic peptide as a triazole group with Lys(N3) group at the 4 position; AA12, AA13 are absent; and Y is NH2 or absent.

In some embodiments, the peptide comprises an amino acid sequence selected from the group consisting of SEQ ID NO: 8, SEQ ID NO: 9, SEQ ID NO: 10, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, and SEQ ID NO: 75; optionally wherein the amino acid sequence is selected from the group consisting of SEQ ID NO: 9, SEQ ID NO: 49, SEQ ID NO: 71, SEQ ID NO: 72, and SEQ ID NO: 73; optionally wherein the peptide comprises the amino acid sequence selected from the group consisting of SEQ ID NO: 49, SEQ ID NO: 72, and SEQ ID NO: 73. In certain embodiments, the peptide comprises the amino acid sequence Seq ID NO: 72.

Disclosed herein are peptides of the formula: Z-XAA1-XAA2-XAA3-XAA4-XAA5-XAA6-XAA7-XAA8-XAA9-XAA10-XAA11-XAA12-XAA13-Y, wherein: (a) Z is absent, or comprises an N-terminus sequence; (b) AA1 is absent, comprises a Ser¹ amino acid, or comprises a Ser¹ substitution moiety; (c) AA2 is absent, comprises a Tyr² amino acid, or comprises a Tyr² substitution moiety; (d) AA3 is absent, comprises a Ser³ amino acid, or comprises a Ser³ substitution moiety; (e) AA4 is absent, comprises a Met⁴ amino acid, or comprises a Met⁴ substitution moiety; (f) AA5 is absent, comprises a Glu⁵ amino acid, or comprises a Glu⁵ substitution moiety; (g) AA6 is absent, comprises a His⁶ amino acid, or comprises a His⁶ substitution moiety; (h) AA7 is absent, comprises a Phe⁷ amino acid, or comprises a Phe⁷ substitution moiety; (i) AA8 is absent, comprises a Arg⁸ amino acid, or comprises a Arg⁸ substitution moiety; (j) AA9 is absent, comprises a Trp⁹ amino acid, or comprises a Trp⁹ substitution moiety; (k) AA10 is absent, comprises a Gly¹⁰ amino acid, or comprises a Gly¹⁰ substitution moiety; (1) AA11 is absent, comprises a Lys¹¹ amino acid, or comprises a Lys¹¹ substitution moiety; (m) AA12 is absent, comprises a Pro¹² amino acid, or comprises a Pro¹² substitution moiety; (n) AA13 is absent, comprises a Val¹³ amino acid, or comprises a Val¹³ substitution moiety; and (o) Y is absent, or comprises a C-terminus sequence.

In some embodiments, Z comprises an N-terminus sequence selected from: Ac, norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethiy-phenyiglycine, 4-isopropyl-phenylglycine, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, (S)-2-amino-3-(1H-indazol-3-yl)propanoic acid, Ac-Nle; Ac-Arg; 7-Ahept; BzlSO₂, PyrPropHep; 2-Nac; Nba; Npa; Pba; Ppa; Tos; or a combination thereof; optionally wherein the N-terminus sequence comprises a string of 5 or 6 amino acids (G/L-G/L-G/L-G/L-G/L-G/L), each independently selected from Glu or Lys; or optionally wherein the N-terminus sequence comprises poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., PLy-PGa-α-MSH).

In some embodiments, AA1 comprises a Ser¹ amino acid, or comprises a Ser¹ substitution moiety selected from: D-Ser, NMe-Ser, Ile, Thr, Tyr, Tyr(Me), or D-stereoisomers thereof; AA2 comprises a Tyr² amino acid, or comprises a Tyr² substitution moiety selected from: D-Tyr, Ile, 2-Nal, 2-Pal, 3-Pal, Phe, 3C1-Phe, 4F-Phe, 4C1-Phe, 4M-Phe, 4T-Phe, Phe(2,4-DiCl), Ser, Thr, Tic, Tyr(Me), or D-stereoisomers thereof; AA3 comprises a Ser³ amino acid, or comprises a Ser³ substitution moiety selected from: D-Ser, Ile, Leu, Nle, Tyr, Val, BrAc, or D-stereoisomers thereof, and optionally forms a cyclic peptide with group at the 9, 10, 11 or 12 position; AA4 comprises a Met⁴ amino acid, or comprises a Met⁴ substitution moiety selected from: Lys, Lys(N3), BrAc, R4-R10, S4-S10, D-Met, Asp, Can, Cba, Cha, Cpna, Cpra, Cys, D-Cys, hCys, D-hCys, Glu, Gly, Hcy, Hle, Ile, Leu, (cyclohexyl)Gly, Nle, Nle(Met), Pen, D-Pen, Ser, Tyr, or Val, or D-stereoisomers thereof and optionally forms a cyclic peptide with group at the 9, 10, 11 or 12 position; AA5 comprises a Glu⁵ amino acid, or comprises a Glu⁵ substitution moiety selected from: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OrnN3, Nle, Orn, Ser, Succ, Tyr, or 4-aminobutyric acid, or D-stereoisomers thereof and optionally forms a cyclic peptide with group at the 9, 10, 11 or 12 position; AA6 comprises a His⁶ amino acid, or comprises a His⁶ substitution moiety selected from: D-His, NMe-His, NMe-D-His, Phe-4-NH2, Aib, Aic, Ala, D-Ala, Arg, Asn, Asp, Cha, Chg, Cit, Cys, D-Cys, Dab, Dap, Gln, Glu, His(1-Me), His(3-Me), Hyp, Hyp(Bzl), Ile, Leu, Lys, Met, Met(O), Met(O2), Nle, D-Nle, Orn, 2-Pal, 3-Pal, 4-Pal, Phe, Pro, Sar, Ser, Ser(Bzl), Thr, Thr(OBzl), Tic, Tle, Trp, Tyr, Tyr(Me), or Val, cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, (3-furyl)alanine, a Phe moiety optionally substituted by halogen, hydroxyl, alkoxy, nitro, benzoyl, methyl, trifluoromethyl, amino, or cyano groups, or D-stereoisomers thereof and optionally forms a cyclic peptide with group at the 9, 10, 11 or 12 position; AA7 comprises a Phe⁷ amino acid, or comprises a Phe⁷ substitution moiety selected from: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tic, Thi, D-Thi, Trp, Tyr, D-Tyr, Tyr(Me), Tyr(Me), D-Tyr(Me), Val, or D-Val, or D-stereoisomers thereof; AA8 comprises a Arg⁸ amino acid, or comprises a Arg⁸ substitution moiety selected from: hArg, norArg, D-Arg, Arg(Me), Arg(Me)2, Ala, Cys, D-Cys, Dab, Dap, Dpr(beta-Ala), Leu, Lys, hLys, Nle, (Nlys)Gly, NMe-Arg, Orn, Phe, Phe(4-Cl), D-Phe(4-Cl), Ser, or Trp, or D-stereoisomers thereof; AA9 comprises a Trp⁹ amino acid, or comprises a Trp⁹ substitution moiety selected from: D-Trp, Trp(6-Me), Trp(7-Me), Aic, Atc, Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq, Tpi, or D-Tpi, R4-R10, S4-S10, Ndab, Orn or D-stereoisomers thereof and optionally forms a cyclic peptide with group at the 3, 4 or 5 position; AA10 comprises a Gly¹⁰ amino acid, or comprises a Gly¹⁰ substitution moiety selected from: D-Gly, Ala, D-Ala, Arg, 6-Ahx, Cys, D-Cys, Daa, Dab, DabN3, Glu, Lys, D-Lys, hLys, LysN3, Orn, Pra, Hpra, Trp, R4-R10, S4-S10, or 5-aminopentanoic acid, or D-stereoisomers thereof and optionally forms a cyclic peptide with group at the 3, 4 or 5 position; AA11 comprises a Lys¹¹ amino acid, or comprises a Lys¹¹ substitution moiety selected from: D-Lys, Ala, Asn, Asp, Cys, D-Cys, Dab, Glu, hGlu, Hgin, Gly, Lys(Me)2, Pra, or Orn, or D-stereoisomers thereof and optionally forms a cyclic peptide with group at the 3, 4 or 5 position; AA12 comprises a Pro¹² amino acid, or comprises a Pro¹² substitution moiety selected from: D-Pro, Ala, Asp, Cys, Gly, Ile, Leu, Met, Phe, Ser, Trp, Val, or D-stereoisomers thereof and optionally forms a cyclic peptide with group at the 3, 4 or 5 position; and/or AA13 comprises a Val¹³ amino acid, or comprises a Val¹³ substitution moiety selected from: D-Val, NMe-Val, Ala, Can, Cba, Cha, Cpna, Cpra, Cys, Hcy, Hle, Ile, Leu, Met, Nle, Pro, or D-stereoisomers thereof.

In some embodiments, Y comprises a C-terminus sequence selected from: NH₂, OH, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, or Trp-NH₂.

In one embodiment, Z is Ac, PBA, octanoyl-PEG8G-G, PyAA, HyBA, HyPA, MoPA, HymBA, C7, C8, or C9; AA1 is Ser or absent; AA2 is Tyr or absent; AA3 is Met or absent; AA4 is Met or Nle or absent; AA5 is Glu; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Trp, or Ala; AA11 is Lys, Gly, Asn, HgIn, Lys(Me)2, or absent; AA12 is Pro or absent; AA13 is Val or absent; and Y is NH2 or absent. In some embodiments, the peptide comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 35.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle or Lys(N3) or optionally Lys(N3) forms a cyclic peptide as a triazole group with Pra group at the 11 position; AA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 10 position; AA11 is absent, Pra or optionally Pra forms a cyclic peptide as a triazole group with Lys(N3) group at the 4 position; AA12, AA13 are absent; and Y is NH2 or absent. In some embodiments, the peptide comprises an amino acid sequence selected from the group consisting of SEQ ID NO: 8, SEQ ID NO: 9, SEQ ID NO: 10, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, and SEQ ID NO: 75.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; AA11, AA12, AA13 are absent; and Y is NH2 or absent. In some embodiments, the peptide comprises an amino acid sequence selected from the group consisting of SEQ ID NO: 8, SEQ ID NO: 9, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, and SEQ ID NO: 75.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; AA11 is Lys, Gly, Asn, HgIn, or Lys(Me)2; AA12, AA13 are absent; and Y is NH2 or absent. In some embodiment, the peptide comprises an amino acid sequence selected from the group consisting of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; AA11, AA12, AA13 are absent; and Y is NH2 or absent. In some embodiments, the peptide comprises an amino acid sequence selected from the group consisting of SEQ ID NO: 38 and SEQ ID NO: 39.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; AA11 is Lys, Gly, Asn, HgIn, or Lys(Me)2; AA12, AA13 are absent; and Y is NH2 or absent.

In one embodiment, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Cys or α-Me-Cys or optionally Cys or α-Me-Cys form a cyclic peptide with Cys or α-Me-Cys group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Cys or α-Me-Cys or optionally Cys or α-Me-Cys form a cyclic peptide with Cys or α-Me-Cys group at the 5 position; AA11 is absent, Lys, Gly, Asn, HgIn, or Lys(Me)2; AA12, AA13 are absent; and Y is NH2 or absent. In some embodiments, the peptide comprises an amino acid sequence of SEQ ID NO: 40.

In one embodiments, Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Dab or optionally Dab forms a cyclic peptide with Dab group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Dab or optionally Dab forms a cyclic peptide with Dab group at the 5 position; AA11 is absent, Lys, Gly, Asn, HgIn, or Lys(Me)2; AA12, AA13 are absent; and Y is NH2 or absent. In some embodiments, the peptide comprises an amino acid sequence of SEQ ID NO: 41.

In some embodiments, the peptide comprises a sequence selected from Table 3.

Disclosed herein are peptides comprising an amino acid sequence selected from the group consisting of SEQ ID NO: 1, SEQ ID NO: 2, SEQ ID NO: 3, and SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, and SEQ ID NO: 75.

Also disclosed herein are α-MSH analogs comprising a peptide described herein.

Further disclosed herein are α-MSH analogs comprising a peptide portion conjugated to a non-peptide portion, wherein the peptide portion comprises a peptide described herein. In some embodiments, the non-peptide portion is selected from: lipids, carbohydrates, small molecules, RNA, DNA, polymers, or combinations thereof. In one embodiment, the non-peptide portion is selected from: a cholesterol oleate moiety, a cholesteryl laurate moiety, an α-tocopherol moiety, a phytol moiety, an oleate moiety, an unsaturated cholesterol-ester moiety, or a lipophilic compound selected from acetanilides, anilides, aminoquinolines, benzhydryl compounds, benzodiazepines, benzofurans, cannabinoids, cyclic polypeptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, or vinca alkaloids. In one embodiment, the non-peptide portion is selected from: polyalkylene oxide homopolymers, polypropylene glycols, polyoxyethylenated polyols and copolymers thereof, polyethylene glycol (PEG), an albumin binding moiety, or a cell penetrating moiety. In one embodiment, the non-peptide portion is selected from: fatty acids, phospholipids, and sterols. In one embodiment, the non-peptide portion is selected from the group consisting of Palm-PEG8-G-G-Ser-Tyr (SEQ ID NO: 169), Ac-K(Palm)-G-G-Ser-Tyr (SEQ ID NO: 170), capric acid (C10), lauric acid (C12), myristic acid (C14), palmitic acid (C16), stearic acid (C18), PEG4, and PEG8.

Also disclosed herein are pharmaceutical compositions comprising an α-MSH analog described herein, and at least one pharmaceutically acceptable excipient.

Further disclosed herein are methods for treating or preventing one or more ophthalmic indications in a subject, the method comprising administering to the subject a therapeutically effective amount of a pharmaceutical composition described herein.

In some embodiments, the one or more ophthalmic indications comprise an ophthalmic indication related to corneal endothelial cells (CEnC) (e.g., related to CEnC loss, abnormal CEnC morphology, CEnC dysfunction, or a combination thereof). In one embodiment, the one or more ophthalmic indications comprise: a corneal injury, a corneal dystrophy, an anterior corneal dystrophy, a stromal corneal dystrophy, a posterior corneal dystrophy, corneal endothelial dystrophy, Fuchs endothelial dystrophy, congenital hereditary endothelial dystrophy, posterior polymorphous corneal dystrophy, and/or Schnyder crystalline corneal dystrophy. In some embodiments, the therapeutically effective amount of the pharmaceutical composition is administered to the subject via topical administration.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 presents the sequence structure for natural α-MSH peptide.

FIG. 2 provides Tables summarizing the experimental design conditions for assessing K110, K150, K172, K173, and K174 in rabbits via topical administration and assessing K150, K172, and K174 in rabbits via subconjunctival injection.

FIG. 3 provides a Table summarizing the experimental design conditions for further assessing the pharmacokinetics of K173 in rabbits via topical administration.

FIG. 4 shows significant amounts of K173 are delivered to target tissues. K173 appears to have a prolonged ocular surface retention and Tₘax after a single dose. Aqueous humor (AH) concentrations were shown to increase with repeated dosing and measurable levels were detected after 24 hours in all dose groups.

FIGS. 5A-5B provides a Table summarizing the experimental design conditions for assessing K173 in rabbits when administered after a corneal injury at various concentrations. FIG. 5A provides the initial experimental design and FIG. 5B provides the updated experimental design adapted for lower dosages for tolerability and efficacy.

FIG. 6 provides a timeline for conducting the study assessing K173 when administered after a corneal injury at various concentrations. After the initial dosages of K173 were administered after cornea injury non-ocular pharmacologic effects were observed and dosing was halted. Upon recovery of the animals dosing was resumed at reduced dosages.

FIGS. 7A-7C present OCT images showing the stromal thickness of the eye of rabbits administered a vehicle or K173 at a dosage of 0.05 mg/mL K173, 0.1 mg/mL K173, or 0.5 mg/mL. Stromal thickness can be used as a measurement of corneal endothelial function and edema. Images were obtained from Day 15 (FIG. 7A) and Day 22 (FIG. 7B). FIG. 7C provides a comparison of vehicle against 0.1 mg/mL K173 at Day 15 and Day 22.

FIG. 8 shows decreased corneal edema in rabbits after administration of K173 at a dosage of 0.05 mg/mL or 0.1 mg/mL. Statistically significant efficacy was seen soon after dosing with the two lowest treatment options when looking at corneal stromal thickness by OCT.

FIG. 9 demonstrates enhanced wound healing by topically administering 0.1 mg/mL K173 to rabbits. Corneal flat mount analysis was performed on 3 animals per group to assess the rate of wound closure.

FIG. 10 provides a Table summarizing the experimental design conditions for assessing the pharmacokinetics of K173 when administered in rats.

FIG. 11 shows the tissue distribution of K173 in rats following 4 days of BID topical dosing. K173 was shown to be present in all parts of the brain, including the hypothalamus, hippocampus, and remaining brain samples, as well as in the aqueous humor and the retina. High levels of K173 were found in the neural retina suggesting both corneal and scleral penetration.

FIGS. 12A-12C depict the structures for each of the K173 (FIG. 12A), K174 (FIG. 12B) and K150 (FIG. 12C) α-MSH analog peptides, respectively. The K173 and K174 α-MSH analog peptides have nearly identical structures and were shown to have very similar ocular distribution patterns. The lack of the HyBA (3,5-hydroxybenzoic acid) residue on the K150 α-MSH analog peptide is likely causing the observed difference in corneal and scleral permeability and may increase melanin binding.

FIGS. 13A-13B presents the in vivo distribution patterns of certain topically administered α-MSH analog peptides (K173, K174, and K150) following their administration to minipigs twice daily for four days. As shown in FIGS. 13A and 13B, the topically administered α-MSH analog peptides K173 and K174 appear to have similar distribution patterns, while K150 appears to have decreased scleral/retinal exposure and enhanced melanin binding.

FIG. 14 presents the in vivo distribution patterns of certain topically administered α-MSH analog peptides (K173, K174 and K150) following their administration to minipigs twice daily for four days. As shown in FIG. 14, the α-MSH analog peptides demonstrate enhanced corneal permeability, can be delivered topically and following which they were detected in the central nervous system tissues of the animals, thereby evidencing that such α-MSH analog peptides can be delivered topically for retinal indications.

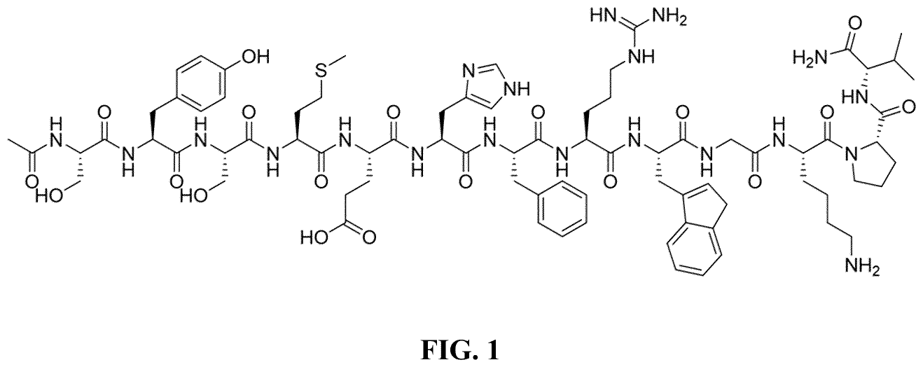

### DETAILED DESCRIPTION

### I. Melanocortin Receptors and α-MSH

### MCR Overview

A family of G-protein-coupled receptors (GPCRs) with seven transmembrane domains have been identified, known as melanocortin receptors (MCRs). Five primary MCR subtypes have been identified in mammals: (1) melanocortin-1 receptors (MC1-R), which are known to be expressed in a range of human cells (including human melanocytes, melanoma cells, CNS gray matter, testis, macrophages, neutrophils, glioma cells, astrocyctes, monocytes, and endothelial cells), and are generally associated with melanin formation (e.g., hair pigmentation, skin pigmentation, etc.) and regulating the immune system (e.g., inflammation); (2) melanocortin-2 receptors (MC2-R), which are expressed primarily in adrenal gland cells and are generally associated with steroid-genesis (e.g., corticosteroid production); (3) melanocortin-3 receptors (MC3-R), which are expressed primarily in CNS cells (such as in the hypothalamus, mid-brain and brainstem) and are generally associated with energy homeostasis, food intake, and inflammation; (4) melanocortin-4 receptors (MC4-R), which are also expressed primarily in CNS cells and are generally associated with feeding behavior, energy homeostasis, and sexual function; and (5) melanocortin-5 receptors (MC5-R), which are broadly expressed in various peripheral tissues and are generally associated with regulation of the exocrine gland system.

MCRs and corresponding melanocortin peptides have also been found to mediate a number of other physiological conditions, including: immunomodulation, motivation, learning, memory, behavior, inflammation, body temperature, pain, perception, blood pressure, heart rate, vascular tone, brain blood flow, nerve growth, placental development, aldosterone synthesis and release, thyroxin release, spermatogenesis, ovarian weight, prolactin and FSH secretion, uterine bleeding in women, sebum and pheromone secretion, blood glucose levels, weight homeostasis, and intrauterine fetal growth (as well as other events surrounding parturition).

Melanocortins are a family of regulatory peptides known to have agonistic and antagonistic binding affinities to MCRs. Natural melanocortins are synthesized by post-translational processing of the hormone propiomelanocortin (POMC—131 amino acids long).

An exemplary amino acid sequence of the human POMC preprotein (NCBI Accession No. NP_001306134.1 and UniProt Accession No. P01189) is as follows:

(SEQ ID NO: 80)
MPRSCCSRSGALLLALLLQASMEVRGWCLESSQCQDLTTESNLLECIRA
CKPDLSAETPMFPGNGDEQPLTENPRKYVMGHFRWDRFGRRNSSSSGSS
GAGQKREDVSAGEDCPLPEGGPEPRSDGAKPGPREGKRSYSMEHFRWGK
PVGKKRRPVKVYPNGAEDESAEAFPLEFKRELTGQRLREGDGPDGPADD
GAGAQADLEHSLLVAAEKKDEGPYRMEHFRWGSPPKDKRYGGFMTSEKS
QTPLVTLFKNAIIKNAYKKGE.

In the POMC amino acid sequence above, positions 1 to 26 correspond to the signal peptide; positions 27 to 102 correspond to the N-terminal peptide of pro-piomelanocortin (NPP); positions 77 to 87 correspond to 7-MSH; positions 105 to 134 correspond to a potential peptide; positions 138 to 176 correspond to corticotropin; positions 138 to 150 correspond to α-MSH; positions 156 to 176 correspond to corticotropin-like intermediary peptide; positions 179 to 267 correspond to lipotropin beta; positions 179 to 234 correspond to lipotropin gamma; positions 217-234 correspond to 3-MSH; positions 237 to 267 correspond to β-endorphin; and positions 237 to 241 correspond to met-enkephalin.

The post-translational processing of POMC produces three primary classes of hormones: melanocortins (MCs), adrenocorticotropin (ACTH), and certain endorphins (e.g., lignotropin). Melanocortins (such as α-MSH, 3-MSH, 7-MSH) are known to have a range of binding affinities and activities for most MCRs, including MC1-R, MC3-R, MC4-R, and MC5-R. Binding to MC2-R is generally limited to adrenocorticotropin (ACTH).

MCRs are viewed as promising therapeutic targets for treating a range of major pathologies and indications, including obesity, diabetes, inflammatory conditions, and sexual dysfunction. However, many melanocortins (and analogs thereof) have limited selectivity between various MCRs and tissue types, with few MCR-specific peptides having been identified as effective therapeutics or approved for pharmaceutical applications. Likewise, the modelling and design of highly selective MCR agonists/antagonists for specific receptor subtypes in specific tissues for specific indications remains a difficult task. A strong need therefore exists for novel MCR-targeting compounds (e.g., novel α-MSH peptide analogs) which are highly specific to particular MCR subtypes, particular tissues, and/or particular therapeutic indications.

In some embodiments, compounds of the present disclosure interact with one or more MCRs to affect their activity (e.g., agonist, antagonist, etc.).

### α-MSH Analogs

The present disclosure presents novel α-MSH peptide analogs. In some embodiments, the novel α-MSH peptide analog is a peptidomimetic of natural α-MSH peptide. In some embodiments, the α-MSH analog contains structural elements that are not found in natural peptides (i.e., peptides comprised of only the 20 proteinogenic amino acids). In some embodiments, the α-MSH peptidomimetic analog contains changes in structural sequence (additions, deletions, substitutions) and/or the presence of amino acids that do not occur in nature, as compared to natural α-MSH peptide. In some embodiments, the α-MSH analog contains one or more fragments of natural α-MSH peptide. In some embodiments, the α-MSH analog can contain one or more of the following: amino acids with side chains that are not found among the known 20 proteinogenic amino acids; non-peptide-based bridging moieties (e.g., cyclic structure, link, staple, bridge, etc.) used to effect cyclization (i.e., conformational constraints) between the ends or internal portions of the molecule; substitutions of the amide bond hydrogen moiety by methyl groups (N-methylation) or other alkyl groups; stereoisomerization between L- and D-amino acids; replacement of a peptide bond with a chemical group or bond that is resistant to chemical or enzymatic treatments; N- and C-terminal modifications; conjugation with a non-peptidic (i.e., non-peptide-based) extensions (such as polyethylene glycol, lipids, carbohydrates, nucleosides, nucleotides, nucleoside bases, various small molecules, or phosphate or sulfate groups); or combinations thereof.

α-MSH is also known as alpha-melanotropin, alpha-melanocortin, and alpha-intermedin. α-MSH is a linear tridecapeptide melanocortin having the following formula: Ac-Ser-Tyr-Ser-Met-Glu-His-Phe-Arg-Trp-Gly-Lys-Pro-Val-NH2 (SEQ ID NO: 76) (IUPAC name: N-acetyl-L-seryl-L-tyrosyl-L-seryl-L-methionyl-L-α-glutamyl-L-histidyl-L-phenylalanyl-L-arginyl-L-tryptophylglycyl-L-lysyl-L-prolyl-L-valinamide) (SEQ ID NO: 76).

Natural alpha-melanocyte stimulating hormone (α-MSH) is as a cleavage product derived from the large precursor protein, proopiomelanocortin (POMC). α-MSH is an endogenous ligand to the melanocortin receptor MC1 (MC1-R), with α-MSH binding to MC1-R at a sub-nanomolar binding affinity. α-MSH also binds to other melanocortin receptors, including MC3-R, MC4-R, and MC5-R; α-MSH does not bind to MC2-R.

Studies of α-MSH, as well as various α-MSH analogs, have shown the heptapeptide sequence of Met⁴-Glu⁵-His⁶-Phe⁷-Arg⁸-Trp⁹-Gly¹⁰ (SEQ ID NO: 81) (i.e., Methionine-Glutamic acid-Histidine-Phenylalanine-Arginine-Tryptophan-Glycine (SEQ ID NO: 81)) to function as a common active core (CAC) within α-MSH-derived peptides. Similar studies of α-MSH analogs have shown the quadrapeptide sequence of His⁶-Phe⁷-Arg⁸-Trp⁹ (SEQ ID NO: 82) (i.e., Histidine-Phenylalanine-Arginine-Tryptophan (SEQ ID NO: 82)) to generally function as the central active core for MC1-R activity.

In some embodiments, the present disclosure presents novel α-MSH peptide analogs that bind with high affinity to one or more melanocortin receptors (MCRs) (e.g., MC1-R). In some embodiments, the present disclosure presents novel α-MSH peptide analogs that bind with high affinity to MC1-R. In some embodiments, the α-MSH analog is an MC1-R preferring ligand. A ligand can be “MC1-R-preferring” in two ways: (1) A high binding preference to MC1-R (e.g., lower EC₅₀ for MC1-R than other MCRs), and/or (2) A higher binding strength to MC1-R. In some embodiments, the α-MSH analog is an MC1-R preferring ligand, and has an EC₅₀ for MC1-R of less than 5%, 10%, 20%, 50% or 100% compared to the EC₅₀ for one or more other MCRs (e.g., as measured by a cAMP assay). In some embodiments, the α-MSH analog is an MC1-R preferring ligand, and has an EC₅₀ for MC1-R of less than 5%, 10%, 20%, 50% or 100% compared to the EC₅₀ for MC2-R, MC3-R, MC4-R, and/or MC5-R (e.g., as measured by a cAMP assay). In some embodiments, the α-MSH analog is an MC1-R preferring ligand, and has an IC₅₀ for MC1-R of less than 5%, 10%, 20%, 50% or 100% compared to the IC₅₀ for one or more other MCRs (e.g., as measured by a competitive binding assay). In some embodiments, the α-MSH analog is an MC1-R preferring ligand, and has an IC₅₀ for MC1-R of less than 5%, 10%, 20%, 50% or 100% compared to the IC₅₀ for MC2-R, MC3-R, MC4-R, and/or MC5-R (e.g., as measured by a competitive assay).

In some embodiments, the present disclosure presents novel α-MSH peptide analogs that bind with high affinity to MC3-R. In some embodiments, the α-MSH analog is an MC3-R preferring ligand. A ligand can be “MC3-R-preferring” in two ways: (1) A high binding preference to MC3-R (e.g., lower EC₅₀ for MC3-R than other MCRs), and/or (2) A higher binding strength to MC3-R. In some embodiments, the α-MSH analog is an MC3-R preferring ligand, and has an EC₅₀ for MC3-R of less than 5%, 10%, 20%, 50% or 100% compared to the EC₅₀ for one or more other MCRs (e.g., as measured by a cAMP assay). In some embodiments, the α-MSH analog is an MC3-R preferring ligand, and has an EC₅₀ for MC3-R of less than 5%, 10%, 20%, 50% or 100% compared to the EC₅₀ for MC1-R, MC2-R, MC4-R, and/or MC5-R (e.g., as measured by a cAMP assay). In some embodiments, the α-MSH analog is an MC3-R preferring ligand, and has an IC₅₀ for MC3-R of less than 5%, 10%, 20%, 50% or 100% compared to the IC₅₀ for one or more other MCRs (e.g., as measured by a competitive binding assay). In some embodiments, the α-MSH analog is an MC3-R preferring ligand, and has an IC₅₀ for MC3-R of less than 5%, 10%, 20%, 50% or 100% compared to the IC₅₀ for MC1-R, MC2-R, MC4-R, and/or MC5-R (e.g., as measured by a competitive assay).

In some embodiments, the present disclosure presents novel α-MSH peptide analogs that bind with high affinity to MC4-R. In some embodiments, the α-MSH analog is an MC4-R preferring ligand. A ligand can be “MC4-R-preferring” in two ways: (1) A high binding preference to MC4-R (e.g., lower EC₅₀ for MC4-R than other MCRs), and/or (2) A higher binding strength to MC4-R. In some embodiments, the α-MSH analog is an MC4-R preferring ligand, and has an EC₅₀ for MC4-R of less than 5%, 10%, 20%, 50% or 100% compared to the EC₅₀ for one or more other MCRs (e.g., as measured by a cAMP assay). In some embodiments, the α-MSH analog is an MC4-R preferring ligand, and has an EC₅₀ for MC4-R of less than 5%, 10%, 20%, 50% or 100% compared to the EC₅₀ for MC1-R, MC2-R, MC3-R, and/or MC5-R (e.g., as measured by a cAMP assay). In some embodiments, the α-MSH analog is an MC4-R preferring ligand, and has an IC₅₀ for MC4-R of less than 5%, 10%, 20%, 50% or 100% compared to the IC₅₀ for one or more other MCRs (e.g., as measured by a competitive binding assay). In some embodiments, the α-MSH analog is an MC4-R preferring ligand, and has an IC₅₀ for MC4-R of less than 5%, 10%, 20%, 50% or 100% compared to the IC₅₀ for MC1-R, MC2-R, MC3-R, and/or MC5-R (e.g., as measured by a competitive assay).

In some embodiments, the present disclosure presents novel α-MSH peptide analogs that bind with high affinity to MC5-R. In some embodiments, the α-MSH analog is an MC5-R preferring ligand. A ligand can be “MC5-R-preferring” in two ways: (1) A high binding preference to MC5-R (e.g., lower EC₅₀ for MC5-R than other MCRs), and/or (2) A higher binding strength to MC5-R. In some embodiments, the α-MSH analog is an MC5-R preferring ligand, and has an EC₅₀ for MC5-R of less than 5%, 10%, 20%, 50% or 100% compared to the EC₅₀ for one or more other MCRs (e.g., as measured by a cAMP assay). In some embodiments, the α-MSH analog is an MC5-R preferring ligand, and has an EC₅₀ for MC5-R of less than 5%, 10%, 20%, 50% or 100% compared to the EC₅₀ for MC1-R, MC2-R, MC3-R, and/or MC4-R (e.g., as measured by a cAMP assay). In some embodiments, the α-MSH analog is an MC5-R preferring ligand, and has an IC₅₀ for MC5-R of less than 5%, 10%, 20%, 50% or 100% compared to the IC₅₀ for one or more other MCRs (e.g., as measured by a competitive binding assay). In some embodiments, the α-MSH analog is an MC5-R preferring ligand, and has an IC₅₀ for MC5-R of less than 5%, 10%, 20%, 50% or 100% compared to the IC₅₀ for MC1-R, MC2-R, MC3-R, and/or MC4-R (e.g., as measured by a competitive assay).

In some embodiments, the present disclosure presents novel α-MSH peptide analogs that bind with high affinity to one or more of MC1-R, MC3-R, MC4-R, MC5-R, or a combination thereof.

In some embodiments, the α-MSH peptide analog binds with high affinity to one or more corneal MCRs (e.g., corneal MC1-R), and has sufficient active properties (e.g., agonist properties, ADME, pK) to be suitable for use in the treatment of corneal endothelial cell loss related diseases. In some embodiments, the α-MSH peptide analog binds with high affinity to corneal MC1-R, and has sufficient active properties (e.g., agonist properties, ADME, pK) to be suitable for use in the treatment of corneal endothelial cell loss related diseases.

In some embodiments, the α-MSH peptide analog binds with high affinity to one or more MCRs found in the ocular tissues, and has sufficient active properties (e.g., agonist properties, ADME, pK) to be suitable for use in the treatment of ocular disorders or diseases. In some embodiments, the α-MSH peptide analog binds with high affinity to one or more corneal MCRs, and has sufficient active properties (e.g., agonist properties, ADME, pK) to be suitable for use in the treatment of corneal epithelial cell loss related diseases. In some embodiments, the α-MSH peptide analog binds with high affinity to one or more retinal MCRs, and has sufficient active properties (e.g., agonist properties, ADME, pK) to be suitable for use in the treatment of retinal cell loss related diseases.

In some embodiments, α-MSH analogs of the present disclosure comprise one or more modifications (e.g., substitution, addition, deletion) to the peptide sequence of natural α-MSH. In some embodiments, α-MSH analogs of the present disclosure can comprise one or more modifications (e.g., substitution, addition, deletion) to the peptide sequence of natural α-MSH, as described in U.S. Pat. Nos. 4,457,864, 4,485,039, 5,683,981, 5,714,576, 5,731,408, 6,051,555, 6,054,556, 6,284,735, 6,350,430, 6,476,187, 6,534,503, 6,579,968, 6,600,015, 6,693,165, 6,699,873, 6,887,846, 6,960,646, 6,951,916, 7,045,591, 7,049,398, 7,084,111, 7,307,063, 7,342,089, 7,345,144, 7,368,433, 7,385,025, 7,417,027, 7,517,854, 7,550,602, 7,582,610, 7,601,753, 7,662,782, 7,897,721, 7,795,378, 8,247,530, 8,349,797, 8,703,702, 9,040,663, 9,273,098, 9,850,280, 9,951,116, U.S. Ser. No. 10/106,578, U.S. Ser. No. 10/238,758, U.S. Ser. No. 10/632,171, U.S. Ser. No. 10/660,939, U.S. Ser. No. 10/711,039, U.S. Ser. No. 10/857,208, US 20020143141, US 20030064921, US 20050130901, US 20050187164, US 20060105951, US 20060122121, US 20060293223, US 20070027091, US 20070105759, US 20070123453, US 20070155660, US 20070244054, US 20080039387, US 20080177036, US 20080207493, US 20080280820, US 20080306008, US 20090203581, US 20090305960, US 20110098213, US 20140050773, US 20150297672, WO 1998027113, WO 1999054358, WO 2000005263, WO 2001030808, WO 2001085930, WO 2001090140, WO 2002026774, WO 2005079574, WO 2009006141, WO 2009144432, WO 2009144433, WO 2010065799, WO 2010065800, WO 2010065801, WO 2010065802, WO 2019183472, and WO 2020060983; the contents of which are each incorporated herein by reference in their entirety, as related to α-MSH analogs, and synthesis, use, and formulations thereof.

### Fragments and Substitutions

In some embodiments, α-MSH analogs of the present disclosure can comprise one or more fragments of the peptide sequence of natural α-MSH. In some embodiments, α-MSH analogs of the present disclosure can comprise a deletional variant of natural α-MSH. In some embodiments, α-MSH analogs of the present disclosure can comprise a truncated variant of natural α-MSH. In some embodiments, α-MSH analogs of the present disclosure can comprise a deletional and truncated variant of natural α-MSH. In some embodiments, α-MSH analogs of the present disclosure can comprise one or more fragments of the peptide sequence of natural α-MSH, as shown in Table 1.

TABLE 1
α-MSH analogs - Fragments

| | | | | | | | | | | | | | | | SEQ |
|---|---|---|---|---|---|---|---|---|---|---|---|---|---|---|---|
| N- | | | | | | | | | | | | | | C- | ID |
| term | AA1 | AA2 | AA3 | AA4 | AA5 | AA6 | AA7 | AA8 | AA9 | AA10 | AA11 | AA12 | AA13 | term | NO: |
| Ac | Ser | Tyr | Ser | Met | Glu | His | Phe | Arg | Trp | Gly | Lys | Pro | Val | NH2 | 76 |
| Ac | Ser | Tyr | Ser | Met | Glu | His | Phe | Arg | Trp | Gly | Lys | Pro | | NH2 | 5 |
| Ac | Ser | Tyr | Ser | Met | Glu | His | Phe | Arg | Trp | Gly | Lys | | | NH2 | 6 |
| Ac | Ser | Tyr | Ser | Met | Glu | His | Phe | Arg | Trp | Gly | | | | NH2 | 83 |
| Ac | Ser | Tyr | Ser | Met | Glu | His | Phe | Arg | Trp | | | | | NH2 | 84 |
| Ac | | Tyr | Ser | Met | Glu | His | Phe | Arg | Trp | Gly | Lys | Pro | Val | NH2 | 1 |
| Ac | | Tyr | Ser | Met | Glu | His | Phe | Arg | Trp | Gly | Lys | Pro | | NH2 | 85 |
| Ac | | Tyr | Ser | Met | Glu | His | Phe | Arg | Trp | Gly | Lys | | | NH2 | 86 |
| Ac | | Tyr | Ser | Met | Glu | His | Phe | Arg | Trp | Gly | | | | NH2 | 87 |
| Ac | | Tyr | Ser | Met | Glu | His | Phe | Arg | Trp | | | | | NH2 | 88 |
| Ac | | | Ser | Met | Glu | His | Phe | Arg | Trp | Gly | Lys | Pro | Val | NH2 | 2 |
| Ac | | | Ser | Met | Glu | His | Phe | Arg | Trp | Gly | Lys | Pro | | NH2 | 89 |
| Ac | | | Ser | Met | Glu | His | Phe | Arg | Trp | Gly | Lys | | | NH2 | 90 |
| Ac | | | Ser | Met | Glu | His | Phe | Arg | Trp | Gly | | | | NH2 | 91 |
| Ac | | | Ser | Met | Glu | His | Phe | Arg | Trp | | | | | NH2 | 92 |
| Ac | | | | Met | Glu | His | Phe | Arg | Trp | Gly | Lys | Pro | Val | NH2 | 3 |
| Ac | | | | Met | Glu | His | Phe | Arg | Trp | Gly | Lys | Pro | | NH2 | 93 |
| Ac | | | | Met | Glu | His | Phe | Arg | Trp | Gly | Lys | | | NH2 | 94 |
| Ac | | | | Met | Glu | His | Phe | Arg | Trp | Gly | | | | NH2 | 95 |
| Ac | | | | Met | Glu | His | Phe | Arg | Trp | | | | | NH2 | 96 |
| Ac | | | | | Glu | His | Phe | Arg | Trp | Gly | Lys | Pro | Val | NH2 | 4 |
| Ac | | | | | Glu | His | Phe | Arg | Trp | Gly | Lys | Pro | | NH2 | 7 |
| Ac | | | | | Glu | His | Phe | Arg | Trp | Gly | Lys | | | NH2 | 97 |
| Ac | | | | | Glu | His | Phe | Arg | Trp | Gly | | | | NH2 | 98 |
| Ac | | | | | Glu | His | Phe | Arg | Trp | | | | | NH2 | 99 |
| Ac | | | | | | His | Phe | Arg | Trp | Gly | Lys | Pro | Val | NH2 | 100 |
| Ac | | | | | | His | Phe | Arg | Trp | Gly | Lys | Pro | | NH2 | 101 |
| Ac | | | | | | His | Phe | Arg | Trp | Gly | Lys | | | NH2 | 102 |
| Ac | | | | | | His | Phe | Arg | Trp | Gly | | | | NH2 | 103 |
| Ac | | | | | | His | Phe | Arg | Trp | | | | | NH2 | 104 |

### Ser¹

In some embodiments, α-MSH analogs of the present disclosure can comprise one or more modifications (e.g., substitution, addition, deletion) to the Ser¹ amino acid of the peptide sequence, as compared to natural α-MSH (or fragments thereof, e.g., as in Table 1). In some embodiments, α-MSH analogs of the present disclosure can comprise a deletion of the Ser¹ amino acid of the peptide sequence, as compared to natural α-MSH (or fragments thereof, e.g., as in Table 1). In some embodiments, α-MSH analogs of the present disclosure can comprise a substitution of the Ser¹ amino acid with one or more of the following moieties: D-Ser, NMe-Ser, Ile, Thr, Tyr, or Tyr(Me), or D-stereoisomers thereof.

### Tyr²

In some embodiments, α-MSH analogs of the present disclosure can comprise one or more modifications (e.g., substitution, addition, deletion) to the Tyr² amino acid of the peptide sequence, as compared to natural α-MSH (or fragments thereof, e.g., as in Table 1). In some embodiments, α-MSH analogs of the present disclosure can comprise a deletion of the Tyr² amino acid of the peptide sequence, as compared to natural α-MSH (or fragments thereof, e.g., as in Table 1). In some embodiments, α-MSH analogs of the present disclosure can comprise a deletion of the Ser¹ and Tyr² amino acids of the peptide sequence, as compared to natural α-MSH (or fragments thereof, e.g., as in Table 1). In some embodiments, α-MSH analogs of the present disclosure can comprise a substitution of the Tyr² amino acid with one or more of the following moieties: D-Tyr, Ile, 1-Nal, 2-Nal, 2-Pal, 3-Pal, D-Phe, 3C1-Phe, 4F-Phe, 4C1-Phe, D-Phe (2,4-DiCl), Ser, Thr, Tic, or Tyr(Me), or D-stereoisomers thereof. In some embodiments, α-MSH analogs of the present disclosure can comprise a substitution of the Tyr² amino acid with a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof.

### Ser³

In some embodiments, α-MSH analogs of the present disclosure can comprise one or more modifications (e.g., substitution, addition, deletion) to the Ser³ amino acid of the peptide sequence, as compared to natural α-MSH (or fragments thereof, e.g., as in Table 1). In some embodiments, α-MSH analogs of the present disclosure can comprise a deletion of the Ser³ amino acid of the peptide sequence, as compared to natural α-MSH (or fragments thereof, e.g., as in Table 1). In some embodiments, α-MSH analogs of the present disclosure can comprise a deletion of the Ser¹, Tyr², and Ser³ amino acids of the peptide sequence, as compared to natural α-MSH (or fragments thereof, e.g., as in Table 1). In some embodiments, α-MSH analogs of the present disclosure can comprise a substitution of the Ser³ amino acid with one or more of the following moieties: BrAc, D-Ser, Ile, Leu, Nle, Tyr, or Val, or D-stereoisomers thereof and optionally forms a cyclic peptide with suitable chemical groups at the 9, 10, 11 or 12 position.

### Met⁴

In some embodiments, α-MSH analogs of the present disclosure can comprise one or more modifications (e.g., substitution, addition, deletion) to the Met⁴ amino acid of the peptide sequence, as compared to natural α-MSH (or fragments thereof, e.g., as in Table 1). In some embodiments, α-MSH analogs of the present disclosure can comprise a deletion of the Met⁴ amino acid of the peptide sequence, as compared to natural α-MSH (or fragments thereof, e.g., as in Table 1). In some embodiments, α-MSH analogs of the present disclosure can comprise a deletion of the Ser¹, Tyr², Ser³, and Met⁴ amino acids of the peptide sequence, as compared to natural α-MSH (or fragments thereof, e.g., as in Table 1). In some embodiments, α-MSH analogs of the present disclosure can comprise a substitution of the Met⁴ amino acid with one or more of the following moieties: Lys, Lys(N3), BrAc, R4-R10, S4-S10,D-Met, Asp, Can, Cba, Cha, Cpna, Cpra, Cys, D-Cys, hCys, D-hCys, Glu, Gly, Hle, Ile, Leu, (cyclohexyl)Gly, Nle, Nle(Met), Pen, D-Pen, Ser, Tyr, or Val, or D-stereoisomers thereof and optionally forms a cyclic peptide with suitable chemical groups at the 9, 10, 11 or 12 position.

### Glu⁵

In some embodiments, α-MSH analogs of the present disclosure can comprise one or more modifications (e.g., substitution, addition, deletion) to the Glu⁵ amino acid of the peptide sequence, as compared to natural α-MSH (or fragments thereof, e.g., as in Table 1). In some embodiments, α-MSH analogs of the present disclosure can comprise a deletion of the Glu⁵ amino acid of the peptide sequence, as compared to natural α-MSH (or fragments thereof, e.g., as in Table 1). In some embodiments, α-MSH analogs of the present disclosure can comprise a deletion of the Ser¹, Tyr², Ser³, Met⁴, and Glu⁵ amino acids of the peptide sequence, as compared to natural α-MSH (or fragments thereof, e.g., as in Table 1). In some embodiments, α-MSH analogs of the present disclosure can comprise a substitution of the Glu⁵ amino acid with one or more of the following moieties: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OrnN3, Nle, Orn, Ser, Succ, Tyr, or 4-aminobutyric acid, or D-stereoisomers thereof and optionally forms a cyclic peptide with suitable chemical groups at the 9, 10, 11 or 12 position.

### His⁶

In some embodiments, α-MSH analogs of the present disclosure can comprise one or more modifications (e.g., substitution, addition, deletion) to the His⁶ amino acid of the peptide sequence, as compared to natural α-MSH (or fragments thereof, e.g., as in Table 1). In some embodiments, α-MSH analogs of the present disclosure can comprise a substitution of the His⁶ amino acid with one or more of the following moieties: D-His, NMe-His, NMe-D-His, Phe-4-NH2, Aib, Aic, Ala, D-Ala, Arg, Asn, Asp, Cha, Chg, Cys, D-Cys, Dab, Dap, Gln, Glu, His(1-Me), His(3-Me), Hyp, Hyp(Bzl), Ile, Leu, Lys, Met, Nle, D-Nle, Orn, 2-Pal, 3-Pal, 4-Pal, Phe, Pro, Sar, Ser, Ser(Bzl), Thr, Thr(OBzl), Tic, Tle, Trp, Tyr, Tyr(Me), or Val, or D-stereoisomers thereof and optionally forms a cyclic peptide with suitable chemical groups at the 9, 10, 11 or 12 position.

In some embodiments, α-MSH analogs of the present disclosure can comprise a substitution of the His⁶ amino acid with one or more of the following moieties: NMe-His, NMe-D-His, Phe-4-NH2, Tyr(Me), cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, or (3-furyl)alanine. In some embodiments, α-MSH analogs of the present disclosure can comprise a substitution of the His⁶ amino acid with a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof.

### Phe⁷

In some embodiments, α-MSH analogs of the present disclosure can comprise one or more modifications (e.g., substitution, addition, deletion) to the Phe⁷ amino acid of the peptide sequence, as compared to natural α-MSH (or fragments thereof, e.g., as in Table 1). In some embodiments, α-MSH analogs of the present disclosure can comprise a substitution of the Phe⁷ amino acid with one or more of the following moieties: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tic, Thi, D-Thi, Trp, Tyr, D-Tyr, Tyr(Me), D-Tyr(Me), Val, or D-Val, or D-stereoisomers thereof. In some embodiments, α-MSH analogs of the present disclosure can comprise a substitution of the Phe⁷ amino acid with a D-Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Arg²

In some embodiments, α-MSH analogs of the present disclosure can comprise one or more modifications (e.g., substitution, addition, deletion) to the Arg⁸ amino acid of the peptide sequence, as compared to natural α-MSH (or fragments thereof, e.g., as in Table 1). In some embodiments, α-MSH analogs of the present disclosure can comprise a substitution of the Arg⁸ amino acid with one or more of the following moieties: hArg, norArg, D-Arg, Arg(Me), Arg(Me)2, Ala, Cys, D-Cys, Dab, Dap, Dpr(beta-Ala), Leu, Lys, hLys, Nle, (Nlys)Gly, NMe-Arg, Orn, Phe, Phe(4-Cl), D-Phe(4-Cl), Ser, or Trp, or D-stereoisomers thereof. In some embodiments, α-MSH analogs of the present disclosure can comprise a substitution of the Arg⁸ amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Trp⁹

In some embodiments, α-MSH analogs of the present disclosure can comprise one or more modifications (e.g., substitution, addition, deletion) to the Trp⁹ amino acid of the peptide sequence, as compared to natural α-MSH (or fragments thereof, e.g., as in Table 1). In some embodiments, α-MSH analogs of the present disclosure can comprise a substitution of the Trp⁹ amino acid with one or more of the following moieties: D-Trp, Trp(6-Me), Trp(7-Me), Aic, Atc, Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq, Tpi, or D-Tpi, R4-R10, S4-S10, Ndab, Orn, or D-stereoisomers thereof. In some embodiments, α-MSH analogs of the present disclosure can comprise a substitution of the Trp⁹ amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof and optionally forms a cyclic peptide with suitable chemical groups at the 3, 4 or 5 position.

### Gly¹⁰

In some embodiments, α-MSH analogs of the present disclosure can comprise one or more modifications (e.g., substitution, addition, deletion) to the Gly¹⁰ amino acid of the peptide sequence, as compared to natural α-MSH (or fragments thereof, e.g., as in Table 1). In some embodiments, α-MSH analogs of the present disclosure can comprise a deletion of the Gly¹⁰ amino acid of the peptide sequence, as compared to natural α-MSH (or fragments thereof, e.g., as in Table 1). In some embodiments, α-MSH analogs of the present disclosure can comprise a deletion of the Gly¹⁰, Lys¹¹, Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Gly¹⁰ amino acid with one or more of the following moieties: D-Gly, Ala, D-Ala, Arg, 6-Ahx, Cys, D-Cys, Daa, Dab, DabN3, Glu, Lys, D-Lys, hLys, LysN3, Orn, Pra, Hpra, Trp, R4-R10, S4-S10, or 5-aminopentanoic acid, or D-stereoisomers thereof and optionally forms a cyclic peptide with suitable chemical groups at the 3, 4 or 5 position.

### Lys¹¹

In some embodiments, α-MSH analogs of the present disclosure can comprise one or more modifications (e.g., substitution, addition, deletion) to the Lys¹¹ amino acid of the peptide sequence, as compared to natural α-MSH (or fragments thereof, e.g., as in Table 1). In some embodiments, α-MSH analogs of the present disclosure can comprise a deletion of the Lys¹¹ amino acid of the peptide sequence, as compared to natural α-MSH (or fragments thereof, e.g., as in Table 1). In some embodiments, α-MSH analogs of the present disclosure can comprise a deletion of the Lys¹¹, Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Lys¹¹ amino acid with one or more of the following moieties: D-Lys, Ala, Asn, Asp, Cys, D-Cys, Dab, Glu, hGlu, Hgln, Gly, Lys(Me)2, Pra, or Orn, or D-stereoisomers thereof and optionally forms a cyclic peptide with suitable chemical groups at the 3, 4 or 5 position.

### Pro¹²

In some embodiments, α-MSH analogs of the present disclosure can comprise one or more modifications (e.g., substitution, addition, deletion) to the Pro¹² amino acid of the peptide sequence, as compared to natural α-MSH (or fragments thereof, e.g., as in Table 1). In some embodiments, α-MSH analogs of the present disclosure can comprise a deletion of the Pro¹² amino acid of the peptide sequence, as compared to natural α-MSH (or fragments thereof, e.g., as in Table 1). In some embodiments, α-MSH analogs of the present disclosure can comprise a deletion of the Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Pro¹² amino acid with one or more of the following moieties: D-Pro, Ala, D-Ala, Asp, Cys, D-Cys, Gly, D-Gly, Ile, D-Ile, Leu, D-Leu, Met, D-Met, Phe, D-Phe, Ser, Trp, D-Trp, Val, or D-Val, or D-stereoisomers thereof and optionally forms a cyclic peptide with suitable chemical groups at the 3, 4 or 5 position. In some embodiments, α-MSH analogs of the present disclosure can comprise a substitution of the Pro¹² amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Val¹³

In some embodiments, α-MSH analogs of the present disclosure can comprise one or more modifications (e.g., substitution, addition, deletion) to the Val¹³ amino acid of the peptide sequence, as compared to natural α-MSH (or fragments thereof, e.g., as in Table 1). In some embodiments, α-MSH analogs of the present disclosure can comprise a deletion of the Val¹³ amino acid of the peptide sequence, as compared to natural α-MSH (or fragments thereof, e.g., as in Table 1). In some embodiments, α-MSH analogs of the present disclosure can comprise a substitution of the Val¹³ amino acid with one or more of the following moieties: D-Val, Nme-Val, Ala, D-Ala, Can, Cba, Cha, Cpna, Cpra, Cys, D-Cys, Hcy, Hle, Ile, D-Ile, Leu, D-Leu, Met, D-Met, Nle, Pro, or D-Pro, or D-stereoisomers thereof.

### Terminal Modifications and Conjugations

In some embodiments, α-MSH analogs of the present disclosure can comprise one or more modifications (e.g., substitution, addition, deletion) to one or more terminus (e.g., N-terminus, C-terminus, or both) of the peptide sequence, as compared to natural α-MSH (or fragments thereof, e.g., as in Table 1). In some embodiments, α-MSH analogs of the present disclosure can comprise one or more modifications (e.g., substitution, addition, deletion) to the N-terminus of the peptide sequence, as compared to natural α-MSH (or fragments thereof, e.g., as in Table 1). In some embodiments, α-MSH analogs of the present disclosure can comprise one or more modifications (e.g., substitution, addition, deletion) to the C-terminus of the peptide sequence, as compared to natural α-MSH (or fragments thereof, e.g., as in Table 1). In some embodiments, α-MSH analogs of the present disclosure can comprise one or more modifications (e.g., substitution, addition, deletion) to both the N-terminus of the peptide sequence and the C-terminus of the peptide sequence, as compared to natural α-MSH (or fragments thereof, e.g., as in Table 1).

In some embodiments, α-MSH analogs of the present disclosure can comprise modifications to the N-terminus of the peptide sequence with one or more peptide-based moieties. In some embodiments, α-MSH analogs of the present disclosure can comprise modifications to the C-terminus of the peptide sequence with one or more peptide-based moieties. In some embodiments, α-MSH analogs of the present disclosure can comprise modifications to both the N-terminus of the peptide sequence and the C-terminus of the peptide sequence with one or more peptide-based moieties.

In some embodiments, α-MSH analogs of the present disclosure can comprise modifications to the N-terminus of the peptide sequence with one or more non-peptide-based moieties. In some embodiments, α-MSH analogs of the present disclosure can comprise modifications to the C-terminus of the peptide sequence with one or more non-peptide-based moieties. In some embodiments, α-MSH analogs of the present disclosure can comprise modifications to both the N-terminus of the peptide sequence and the C-terminus of the peptide sequence with one or more non-peptide-based moieties.

In some embodiments, one or more terminal portions of a peptide can be connected to the parent portion peptide using a linking moiety. In some embodiments, the terminal portion can be separated from the parent peptide by 1, 2, 3, 4, 5, 6, 7, 8, 9, 10 or more residues. In some embodiments, the terminal portion can comprise any natural or unnatural amino acid, the N-methylated form of any natural or unnatural amino acid, and/or the D-stereoisomer of any natural or unnatural amino acid. In some embodiments, the terminal portion can be comprise norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and/or (S)-2-amino-3-(1H-indazol-3-yl)propanoic acid.

In some embodiments, α-MSH analogs of the present disclosure can comprise modifications to the N-terminus of the peptide sequence with one or more of the following moieties: Ac-Nle; Ac-Arg; 7-Ahept; BzlSO₂, HyBA; HyPA; HymBA; MoPA, PyrPropHep; 2-Nac; Nba; Npa; Pba; Ppa; PyAA; or Tos.

In some embodiments, α-MSH analogs of the present disclosure can comprise modifications to the N-terminus which comprise a string of 5 or 6 Glu amino acids. In some embodiments, α-MSH analogs of the present disclosure can comprise modifications to the N-terminus which comprise a string of 5 or 6 Lys amino acids. In some embodiments, α-MSH analogs of the present disclosure can comprise modifications to the N-terminus which comprise a string of 5 or 6 amino acids, each independently selected from Glu or Lys. In some embodiments, α-MSH analogs of the present disclosure can comprise modifications to the N-terminus which comprise a string of 5 or 6 amino acids, each independently selected from Glu or Lys, comprising: L-L-L-L-L-L (SEQ ID NO: 105); G-L-L-L-L-L (SEQ ID NO: 106); L-G-L-L-L-L (SEQ ID NO: 107); L-L-G-L-L-L (SEQ ID NO: 108); L-L-L-G-L-L (SEQ ID NO: 109); L-L-L-L-G-L (SEQ ID NO: 110); L-L-L-L-L-G (SEQ ID NO: 111); G-G-L-L-L-L (SEQ ID NO: 112); G-L-G-L-L-L (SEQ ID NO: 113); G-L-L-G-L-L (SEQ ID NO: 114); G-L-L-L-G-L (SEQ ID NO: 115); G-L-L-L-L-G (SEQ ID NO: 116); L-G-G-L-L-L (SEQ ID NO: 117); L-G-L-G-L-L (SEQ ID NO: 118); L-G-L-L-G-L (SEQ ID NO: 119); L-G-L-L-L-G (SEQ ID NO: 120); L-L-G-G-L-L (SEQ ID NO: 121); L-L-G-L-G-L (SEQ ID NO: 122); L-L-G-L-L-G (SEQ ID NO: 123); L-L-L-G-G-L (SEQ ID NO: 124); L-L-L-G-L-G (SEQ ID NO: 125); L-L-L-L-G-G (SEQ ID NO: 126); G-G-G-L-L-L (SEQ ID NO: 127); G-G-L-G-L-L (SEQ ID NO: 128); G-G-L-L-G-L (SEQ ID NO: 129); G-G-L-L-L-G (SEQ ID NO: 130); G-L-G-G-L-L (SEQ ID NO: 131); G-L-G-L-G-L (SEQ ID NO: 132); G-L-G-L-L-G (SEQ ID NO: 133); G-L-L-G-G-L (SEQ ID NO: 134); G-L-L-G-L-G (SEQ ID NO: 135); G-L-L-L-G-G (SEQ ID NO: 136); L-L-L-G-G-G (SEQ ID NO: 137); L-L-G-L-G-G (SEQ ID NO: 138); L-L-G-G-L-G (SEQ ID NO: 139); L-L-G-G-G-L (SEQ ID NO: 140); L-G-L-L-G-G (SEQ ID NO: 141); L-G-L-G-L-G (SEQ ID NO: 142); L-G-L-G-G-L (SEQ ID NO: 143); L-G-G-L-L-G (SEQ ID NO: 144); L-G-G-L-G-L (SEQ ID NO: 145); L-G-G-G-L-L (SEQ ID NO: 146); L-L-G-G-G-G (SEQ ID NO: 147); L-G-L-G-G-G (SEQ ID NO: 148); L-G-G-L-G-G (SEQ ID NO: 149); L-G-G-G-L-G (SEQ ID NO: 150); L-G-G-G-G-L (SEQ ID NO: 151); G-L-L-G-G-G (SEQ ID NO: 152); G-L-G-L-G-G (SEQ ID NO: 153); G-L-G-G-L-G (SEQ ID NO: 154); G-L-G-G-G-L (SEQ ID NO: 155); G-G-L-L-G-G (SEQ ID NO: 156); G-G-L-G-L-G (SEQ ID NO: 157); G-G-L-G-G-L (SEQ ID NO: 158); G-G-G-L-L-G (SEQ ID NO: 159); G-G-G-L-G-L (SEQ ID NO: 160); G-G-G-G-L-L (SEQ ID NO: 161); L-G-G-G-G-G (SEQ ID NO: 162); G-L-G-G-G-G (SEQ ID NO: 163); G-G-L-G-G-G (SEQ ID NO: 164); G-G-G-L-G-G (SEQ ID NO: 165); G-G-G-G-L-G (SEQ ID NO: 166); G-G-G-G-G-L (SEQ ID NO: 167); or G-G-G-G-G-G (SEQ ID NO: 168).

In some embodiments, α-MSH analog compounds of the present disclosure comprise an N-terminal peptide consisting of a chain of about 15 to about 400 identical amino acids. In some embodiments, the N-terminal peptide comprises about 25 to about 300 identical amino acids, about 50 to about 200 identical amino acids, about 75 to about 150 identical amino acids, about 90 to about 120 identical amino acids, or about 100 or 110 identical amino acids. In some embodiments, the N-terminal peptide comprises: poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., Ply-PGa-α-MSH).

In some embodiments, α-MSH analogs of the present disclosure can comprise modifications to the C-terminus of the peptide sequence with one or more of the following moieties: NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, Trp-NH₂.

In some embodiments, α-MSH analogs of the present disclosure can comprise a peptide portion conjugated to a non-peptide-based portion. In some embodiments, α-MSH analogs of the present disclosure can comprise a peptide portion conjugated to a non-peptide portion-based selected from lipids, small molecules, RNA, DNA, polymers, or combinations thereof. In some embodiments, conjugates comprise covalent modifications introduced by reacting targeted amino acid residues or the termini of the peptide with an organic derivatizing agent that is capable of reacting with selected side-chains or terminal residues.

In some embodiments, the conjugation process may involve one or more of: PEGylation, lipidation, albumination, biotinylation, desthiobiotinylation, the addition of other peptide tails, or grafting into proteins.

In some embodiments, α-MSH analogs are conjugated to one or more anchors. In some embodiments, α-MSH analogs are conjugated to one or more anchors selected from: cholesterol oleate moiety, cholesteryl laurate moiety, an α-tocopherol moiety, a phytol moiety, an oleate moiety, an unsaturated cholesterol-ester moiety, or a lipophilic compound selected from acetanilides, anilides, aminoquinolines, benzhydryl compounds, benzodiazepines, benzofurans, cannabinoids, cyclic polypeptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids.

In some embodiments, α-MSH analogs are conjugated to a hydrophilic polymer. In some embodiments, the conjugate includes a hydrophilic polymer selected from: polyalkylene oxide homopolymers, polypropylene glycols, polyoxyethylenated polyols and copolymers thereof. In some embodiments, the conjugate includes polyethylene glycol (PEG). In some embodiments, α-MSH analogs are conjugated to an albumin binding polypeptide. In some embodiments, α-MSH analogs are conjugated to cell penetrating polypeptides.

In some embodiments, α-MSH analogs are conjugated to lipidic moiety. In some embodiments, α-MSH analogs are conjugated to a lipidic moiety selected from: fatty acids, phospholipids, and sterols. In some embodiments, α-MSH analogs are directly conjugated to the lipidic moiety. In some embodiments, the lipidic moiety is conjugated to a polypeptide-PEG conjugate. In some embodiments, α-MSH analogs are conjugated to a lipidic moiety selected from Palm-PEG8-G-G-Ser-Tyr (SEQ ID NO: 169); Ac-K(Palm)-G-G-Ser-Tyr (SEQ ID NO: 170), capric acid (C10), lauric acid (C12), myristic acid (C14), palmitic acid (C16), stearic acid (C18), PEG4, or PEG8.

In some embodiments, two or more α-MSH analogs dimerized by conjugation to the same lipidic moiety.

### Cyclic Peptides

In some embodiments, α-MSH analogs of the present disclosure can comprise cyclic peptides having one or more bridging moieties (e.g., cyclic structure, staple, bridge, etc.), as compared to natural α-MSH (or fragments thereof, e.g., as in Table 1). Peptide stapling/bridging is a macrocyclization approach in which peptides are covalently modified through the formation of a chemical linkage (e.g., staple, bridge moiety, etc.) between the side chains of two amino acids. More specifically, peptides are rendered macrocyclic by formation of covalent bonds between atoms present within the linear peptide and atoms of a bridging moiety. Stapling/bridging can be used to constrain peptides into preferred bioactive conformations (reducing conformational flexibility and degrees of rotational freedom), thereby improving affinity for specific receptor targets and improving overall pharmacokinetics. Without being bound by theory, the residues being linked are generally located on the same face of the peptide helix and separated by one, two, or three helical turns (e.g., a first amino acid at position (z) is linked to a second amino acid at position z+4, z+7, or z+11). In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof.

Examples of bridging moieties/peptide staples for use with compounds of the present disclosure include, but are not limited to: Amide-based (e.g., lactam) bridges; aromatic-ring-based bridges; hydrocarbon chains; Alkene-based hydrocarbon bridges (e.g., using Fmoc-S-2-(2′-pentenyl)alanine); Triazole-based Click bridges, such as copper(I)-catalyzed Huisgen 1,3-dipolar cycloaddition reactions between side chain azido and alkynyl moieties (e.g., Fmoc-L-Nle(εN₃) and Fmoc-D-Pra) (see S. Kawamoto, et al., J. Med. Chem. 2012, 55(3), 1137-1146); dialkynyl staples (e.g., 1,4-Diethynylbenzene, diethynylpentane, diethynylamines) for stapling linear diazido-peptides; Sulfide-bonded disulfide, thioether and bis-thioether bridges; Perfluorobenzene bridges; or combinations thereof.

In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some embodiments, the bridging moiety comprises an amide bond produced by the reaction of the side chains of the following pairs of amino acids: lysine and glutamate; lysine and aspartate; ornithine and glutamate; ornithine and aspartate; homolysine and glutamic acid; homolysine and aspartic acid; and other combinations of amino acids, unnatural amino acids or non-amino acid residues comprising a primary amine and a carboxylic acid.

In some embodiments, bridging moieties may comprise bonds formed between residues that may include, but are not limited to (S)-2-amino-5-azidopentanoic acid; (S)-2-aminohept-6-enoic acid; (S)-2-aminopent-4-ynoic acid; (S)-2-aminopent-4-enoic acid; or combinations thereof.

In some embodiments, bridging moieties are formed through cyclization reactions using olefin metathesis.

In some embodiments, the bridging moiety comprises a disulfide bond formed between two thiol containing residues. In some embodiments, the bridging moiety comprises one or more thioether bonds. Such thioether bonds may include those found in cyclo-thioalkyl compounds. These bonds can be formed during a chemical cyclization reaction between chloro acetic acid N-terminal modified groups and cysteine residues. In some embodiments, bridging moieties comprise one or more triazole ring.

In some embodiments, bridging moieties comprise one or more hydrocarbon chains (linear or branched), and/or hydrocarbon rings (cyclic, heterocyclic, aromatic, heteroaromatic). In some embodiments, hydrocarbon bridging moieties may be introduced by reaction with reagents containing multiple reactive halides, including, but not limited to poly(bromomethyl)benzenes, poly(bromomethyl)pyridines, poly(bromomethyl)alkyl benzenes and/or (E)-1,4-dibromobut-2-ene. Examples of Poly(bromomethyl)benzene molecules of the present disclosure can include 1,2-bis(bromomethyl)benzene; 1,3-bis(bromomethyl)benzene; and 1,4-bis(bromomethyl)benzene.

In some embodiments, the thiol group of a cysteine residue is cross-linked with another cysteine residue to form a disulfide bond. In some embodiments, thiol groups of cysteine residues react with bromomethyl groups of poly(bromomethyl)benzene molecules to form stable linkages (see, e.g., Timmerman et al., ChemBioChem (2005) 6:821-824, the contents of which are incorporated herein by reference in their entirety).

In some embodiments, Bis-, tris- and tetrakis(bromomethyl)benzene molecules can be used to generate bridging moieties to produce peptides with one, two or three loops, respectively. Bromomethyl groups of a poly(bromomethyl)benzene molecule may be arranged on the benzene ring on adjacent ring carbons (ortho- or o-), with a ring carbon separating the two groups (meta- or m-) or on opposite ring carbons (para- or p-). In some embodiments, m-bis(bromomethyl)benzene (i.e., m-dibromoxylene), o-bis(bromomethyl)benzene (i.e., o-dibromoxylene) and/or p-bis(bromomethyl)benzene (i.e., p-dibromoxylene) are used to form cyclic peptides. In some embodiments, thiol groups of cysteine residues react with other reagents comprising one or more bromo functional groups to form stable linkages. Such reagents may include, but are not limited to poly(bromomethyl) pyridines (e.g., 2,6-bis(bromomethyl) pyridine), poly(bromomethyl)alkyl benzenes (e.g., 1,2-bis(bromomethyl)-4-alkylbenzene) and/or (E)-1,4-dibromobut-2-ene.

In some embodiments, a side chain amino group and a terminal amino group are cross-linked with disuccinimidyl glutarate (see, e.g., Millward et al., J. Am. Chem. Soc. (2005) 127:14142-14143. In some embodiments, an enzymatic method is used which relies on the reaction between (1) a cysteine and (2) a dehydroalanine or dehydrobutyrine group, catalyzed by a lantibiotic synthetase, to create the thioether bond (see, e.g., Levengood et al., Bioorg. and Med. Chem. Lett. (2008) 18:3025-3028). The dehydro functional group can also be generated chemically by the oxidation of selenium containing amino acid side chains incorporated during translation (see, e.g., Seebeck et al., J. Am. Chem. Soc. 2006).

In some embodiments, bridging moieties comprise an aromatic, 6-membered ring (e.g., benzene). In some embodiments, bridging moieties comprise a heterocyclic, 6-membered ring which includes one nitrogen atoms (e.g., pyridine). In some embodiments, bridging moieties comprise a heterocyclic, 6-membered ring which includes two nitrogen atoms (e.g., pyridazine, pyrimidine, pyrazine). In some embodiments, bridging moieties comprise a heterocyclic, 6-membered ring which includes three nitrogen atoms (e.g., triazanes). In some embodiments, bridging moieties comprise a heterocyclic, 5-membered ring which includes one nitrogen atoms (e.g., pyrrole). In some embodiments, bridging moieties comprise a heterocyclic, 5-membered ring which includes two nitrogen atoms (e.g., imidazole, pyrazole). In some embodiments, bridging moieties comprise a heterocyclic, 5-membered ring which includes three nitrogen atoms (e.g., triazoles).

In some embodiments, α-MSH analogs of the present disclosure can comprise a bridging link at the AA3 locus (i.e., Ser³ location). In some embodiments, α-MSH analogs of the present disclosure can comprise a bridging link at the AA4 locus (i.e., Met⁴ location). In some embodiments, α-MSH analogs of the present disclosure can comprise a bridging link at the AA5 locus (i.e., Glu⁵ location). In some embodiments, α-MSH analogs of the present disclosure can comprise a bridging link at the AA9 locus (i.e., Trp⁹ location). In some embodiments, α-MSH analogs of the present disclosure can comprise a bridging link at the AA10 locus (i.e., Gly¹⁰ location). In some embodiments, α-MSH analogs of the present disclosure can comprise a bridging link at the AA11 locus (i.e., Lys¹¹ location). In some embodiments, α-MSH analogs of the present disclosure can comprise a bridging link at the AA12 locus (i.e., Pro¹² location).

In some embodiments, α-MSH analogs of the present disclosure can comprise a bridging link at the AA3 locus (i.e., Ser³ location) and at the AA12 locus (i.e., Pro¹² location). In some embodiments, α-MSH analogs of the present disclosure can comprise a bridging link at the AA4 locus (i.e., Met⁴ location) and at the AA10 locus (i.e., Gly¹⁰ location). In some embodiments, α-MSH analogs of the present disclosure can comprise a bridging link at the AA4 locus (i.e., Met⁴ location) and at the AA11 locus (i.e., Lys¹¹ location). In some embodiments, α-MSH analogs of the present disclosure can comprise a bridging link combination selected from: [Lys⁴, Pra¹¹], [Cys⁴, Cys¹⁰], [Cys⁴, Cys¹¹], [Maa⁴, Cys¹⁰], [Mpa⁴, Cys¹⁰], [Nle⁴, Gly¹⁰], or [Hcy⁴, Cys¹⁰].

In some embodiments, α-MSH analogs of the present disclosure can comprise a bridging link at the AA5 locus (i.e., Glu⁵ location) and at the AA9 locus (i.e., Trp⁹ location). In some embodiments, α-MSH analogs of the present disclosure can comprise a bridging link at the AA5 locus (i.e., Glu⁵ location) and at the AA10 locus (i.e., Gly¹⁰ location). In some embodiments, α-MSH analogs of the present disclosure can comprise a bridging link at the AA5 locus (i.e., Glu⁵ location) and at the AA11 locus (i.e., Lys¹¹ location). In some embodiments, AA5 may be either a L- or D-amino acid having an omega-amino or carboxyl group in the side chain, for example AA5 may be α,γ-diaminopropionic acid, α,γ-diaminobutyric acid, Orn, Lys, α-aminoadipic acid, α-aminopimelic acid, or higher (i.e., alkyldionic acids containing more than 7 carbons) homologs, Glu or Asp. In some embodiments, AA5 may be Lys(N3), DLys(N3), Ndab, NGlu, Cys, α-MeCys, Pra or Orn(N3). In some embodiments, AA9 may be Dab, NMeTrp, Trp(Me), Orn, Ndab, Cys or α-MeCys, In some embodiments, AA10 may be either a L- or D-amino acid having an omega-amino or carboxyl group in the side chain, for example, AA10 may be diaminopropionic acid, α,γ-diaminobutyric acid, Orn, Lys, α,β-aminoadipic acid, α-aminopimelic acid, or higher homologs, Glu or Asp. In some embodiments, AA10 may be Trp, Pra, Hpra, Cys, Ala, Dab, Dab(N3), Lys(N3) or D-Lys(N3). In some embodiments, AA11 may be a L- or D-amino acid having an omega-amino or carboxyl group in the side chain, for example, AA11 may be α,β-diaminopropionic acid, α,γ-diaminobutyric acid, Orn, Lys, α-aminoadipic acid, α-aminopimelic acid, or higher homologs, Glu or Asp. In some embodiments, AA11 may be Pra, Cys, Gly, Asn, Hgln, or Lys(Me)2. In some embodiments, α-MSH analogs of the present disclosure can comprise a bridging link combination selected from: [Glu⁵, Dab⁹], [Lys⁵, Pra¹⁰], [NGlu⁵, Orn⁹], [Glu⁵, NDab⁹], [NGlu⁵, NDab⁹], [NDab⁵, NDab⁹], [Cys⁵, Cys⁹], [α-Me-Cys⁵, α-Me-Cys⁹], [Dab, Dab¹⁰], [Orn⁵, Pra¹⁰], [Lys⁵, HPra¹⁰], [Pra⁵, Lys¹⁰], [Orn, HPra¹⁰], [D-Lys⁵, Pra¹⁰], [Lys⁵, D-Pra¹⁰], [Asp⁵, Lys¹⁰], [Glu⁵, Lys¹⁰], [Cys⁵, Cys¹⁰], [Cys⁵, Cys¹¹], [Glu⁵, Orn¹⁰][Lys(N3)⁵, Pra¹⁰], [Asp⁵, Lys¹¹], [Glu⁵, Lys¹¹], [Lys⁵, Asp¹¹], or [Suc⁵, Lys¹⁰].

In some embodiments, α-MSH analogs of the present disclosure can comprise a bridging link at the AA6 locus (i.e., His⁶ location) and at the AA9 locus (i.e., Trp⁹ location). In some embodiments, α-MSH analogs of the present disclosure can comprise a bridging link at the AA6 locus (i.e., His⁶ location) and at the AA10 locus (i.e., Gly¹⁰ location). In some embodiments, α-MSH analogs of the present disclosure can comprise a bridging link at the AA6 locus (i.e., His⁶ location) and at the AA11 locus (i.e., Lys¹¹ location). In some embodiments, α-MSH analogs of the present disclosure can comprise a bridging link combination selected from: [Tyr⁶, Trp⁹], [Tyr⁶, Gly¹⁰], [Tyr⁶, Lys¹¹].

In some embodiments, α-MSH analogs of the present disclosure can comprise one or more Lactam-bridges to provide cyclic α-MSH peptide analogs. Examples of Lactam-bridges for use in α-MSH peptide analogs include those presented in U.S. Pat. No. 9,273,098, the contents of which are incorporated herein by reference in their entirety as related to lactam-bridges for use in MCR-targeting compound such as α-MSH peptide analogs.

In some embodiments, α-MSH analogs of the present disclosure can comprise one or more amide-based lactam bridges, carbonyl-based lactam bridges, or combinations thereof. In some embodiments, α-MSH analogs of the present disclosure can comprise a bridging moiety comprising one or more bridging groups selected from: (CH₂)₁₋₆—C(═O)—NH—(CH₂)₁₋₆; (CH₂)₁₋₆—NH—C(═O)—(CH₂)₁₋₆; (CH₂)₁₋₆—C(═O)—(CH₂)₁₋₆; C(═O)—(CH₂)₁₋₆—; (CH₂)₁₋₆—C(═O)—NH—C(═O)—(CH₂)₁₋₆—; (CH₂)₁₋₆—NH—C(═O)—(CH₂)₁₋₆—NH—C(═O)—(CH₂)₁₋₆; (CH₂)₁₋₆—NH—C(═O)—NH—(CH₂)₁₋₆; [Nitrobenzene]-NH—(CH₂)₁₋₆—NH—C(═O)—(CH₂)₁₋₆; [Phenylamine]-NH—(CH₂)₁₋₆—NH—C(═O)—(CH₂)₁₋₆; [Nitrobenzene]-NH—(CH₂)₁₋₆—N(CH₃)—C(═O)—(CH₂)₁₋₆; or [Phenylamine]-NH—(CH₂)₁₋₆— N(CH₃)—C(═O)—(CH₂)₁₋₆;

### Amino Acid Variants and Derivatization

Amino acids, as used herein, include both natural amino acids (i.e., the 20 proteinogenic amino acids) and unnatural amino acids. The term also includes amino acids bearing a conventional amino protecting group (e.g., acetyl or benzyloxycarbonyl), as well as natural and unnatural amino acids protected at the carboxy terminus (e.g., as a (C1-C6) alkyl, phenyl or benzyl ester or amide, or as an alpha-methylbenzyl amide). Other suitable amino and carboxy protecting groups are known to those skilled in the art (see, e.g., Greene, et al., Protecting Groups In Organic Synthesis; second edition, 1991, New York, John Wiley & sons, Inc.). Peptides and/or peptide compositions of the present invention may also include modified amino acids.

Examples of unnatural amino acids useful for the modifying peptides of the present disclosure include, but are not limited to: 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-aminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine, phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid, (S)-2-aminohept-6-enoic acid, (S)-2-aminopent-4-ynoic acid, (S)-2-aminopent-4-enoic acid, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and (S)-2-amino-3-(1H-indazol-3-yl) butanoic acid, 2-(2′MeOphenyl)-2-amino acetic acid, tetrahydro 3-isoquinolinecarboxylic acid, and stereoisomers thereof (including, but not limited, to D and L isomers).

Additional unnatural amino acids useful for the modifying peptides of the present disclosure include fluorinated amino acids (i.e., amino acids with one or more carbon bound hydrogen atoms being replaced by fluorine). Examples of fluorinated amino acids include, but are not limited to: 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

Additional unnatural amino acids useful for the modifying peptides of the present disclosure include amino acids that are disubstituted at the α-carbon. These include amino acids in which the two substituents on the α-carbon are the same, for example α-amino isobutyric acid, and 2-amino-2-ethyl butanoic acid. These also include amino acids where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidinnne-4-carboxylix acid, and stereoisomers thereof.

Additional unnatural amino acids useful for the modifying peptides of the present disclosure include analogs of tryptophan in which the indole ring system is replaced by another 9 or 10 membered bicyclic ring system comprising 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent is independently selected from H, F, Cl, Br, CN, COOR, CONRR′, oxo, OR, and NRR′. Each R and R′ is independently selected from H, C1-C20 alkyl, C1-C20 alkyl-O—C1-C20 alkyl.

In some embodiments, tryptophan analogs useful for the modifying peptides of the present disclosure include 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to, 4-azatryptophan, 7-azatryptophan and 5-azatryptophan), 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the modifying peptides of the present disclosure include amino acids which are: chemically blocked (reversibly or irreversibly); chemically modified on their N-terminal amino group; chemically modified on their side chain groups; or chemically modified in the amide backbone (e.g., N-methylated, D or L stereoisomers). Examples of modified amino acids include: methionine sulfoxide; methionine sulfone; aspartic acid-(beta-methyl ester); N-ethylglycine; and alanine carboxamide.

In some embodiments, examples of unnatural amino acids useful for the modifying peptides of the present disclosure include those listed in Table 2 of US 2011/0172126, the contents of which are incorporated herein by reference in their entirety as related to unnatural amino acids for the modifying peptides.

In some embodiments, amino acids for use in the present disclosure are modified using an organic proteinaceous or non-proteinaceous derivatizing agent. In some embodiments, amino acids for use in the present disclosure are modified using post-translational modification. In some embodiments, modifications are introduced by reacting targeted amino acid residues of the peptide with an organic derivatizing agent that is capable of reacting with selected side-chains or terminal residues. In some embodiments, modifications are introduced by harnessing mechanisms of post-translational modifications that function in selected recombinant host cells. Certain post-translational modifications are the result of the action of recombinant host cells on an expressed peptide. As one example, glutaminyl and asparaginyl residues are frequently post-translationally deamidated to the corresponding glutamyl and aspartyl residues under certain post-translational conditions (e.g., under mildly acidic conditions). Other post-translational modifications include: hydroxylation of proline and lysine; phosphorylation of hydroxyl groups of tyrosinyl, seryl or threonyl residues; and methylation of the alpha-amino groups of lysine, arginine, and histidine side chains (see, e.g., Creighton et al., Proteins: Structure and Molecular Properties, W.H. Freeman & Co., San Francisco, 1983, pp. 79-86).

In some embodiments, amino acid modifications include the bonding of non-proteinaceous polymers to peptides of the present disclosure. Examples of non-proteinaceous polymers include hydrophilic synthetic polymers (i.e., non-natural polymers), such as hydrophilic polyvinyl polymers (e.g., polyvinylalcohol and polyvinylpyrrolidone). The Examples of non-proteinaceous polymers also include polyethylene glycol, polypropylene glycol and polyoxyalkylenes. In some embodiments, amino acid modifications include the bonding of non-proteinaceous polymers to peptides of the present disclosure, as described in U.S. Pat. Nos. 4,640,835, 4,496,689, 4,301,144, 4,670,417, 4,791,192, and 4,179,337; the contents of which are each incorporated herein by reference in their entirety, as related to amino acid modifications for use in the present disclosure.

According to the present disclosure, the following abbreviations have the meanings as given:

TABLE 2
Structure Abbreviations

| Abbreviation | Full Name/Structure |
|---|---|
| Abu | gamma-amino butyric acid |
| 2-Abz | 2-amino benzoic acid |
| 3-Abz | 3-amino benzoic acid |
| 4-Abz | 4-amino benzoic acid |
| Ac-Glu | Acetyl glutamic acid |
| Achc | 1-amino-cyclohexane-1-carboxylic acid |
| Ac-Lys(N3) | Acetyl azidolysine |
| Ac-Met | Acetyl methionine |
| Ac-Nle | Acetyl norleucine |
| Acpc | 1-amino-cyclopropane-1-carboxylic acid |
| Ac-R5 | Acetyl R-2-(4-pentenyl)alanine |
| Ac-R8 | Acetyl R-2-(7-octenyl) alanine |
| Ac-S5 | Acetyl S-2-(4-pentenyl) alanine |
| Ac-Ser | Acetyl serine |
| Ac-Tyr | Acetyl tyrosine |
| 12-Ado | 12-amino-dodecanoic acid |
| Agp | 2-amino-3-guanidino-propionic acid |
| 7-Ahept | 7-amino-heptanoic acid |
| Aib | alpha-aminoisobutyric acid |
| Aic | 2-aminoindane-2-carboxylic acid |
| 6-Ahx | 6-amino-hexanoic acid |
| alphaMeCys | Alpha methyl cysteine |
| Amb | 4-(aminomethyl)-benzoic acid |
| Amc | 4-(aminomethyl)-cyclohexane-carboxylic acid |
| 8-Aoc | 8-amino-octanoic acid |
| Arg(Tos) | para-tosyl-arginine |
| Arg(Me) | Methyl-arginine |
| ArgMe2 | N_{ω},N′_{ω}-dimethyl-L-arginine |
| Asn-NH2 | Asparagine amide |
| Asp(anilino) | beta-anilino-aspartic acid |
| Asp(3-Cl-anilino) | beta-(3-chloro-anilino)-aspartic acid |
| Asp(3,5-diCl- | beta-(3,5-dichloro anilino)-aspartic acid |
| anilino) | |
| Atc | 2-aminotetralin-2-carboxylic acid |
| 11-Aun | 11-amino-undecanoic acid |
| AVA | 5-amino-valeric acid |
| BzlSO₂ | benzosulfonyl group |
| Bip | biphenyl-alanine |
| BrAc-Glu | Bromoacetyl glutamic acid |
| BrAc-Nle | Bromoacetyl norleucine |
| Can | cyclic analog |
| Cba | cyclobutyl-alanine |
| Cha | cyclohexyl-alanine |
| Chg | cyclohexyl-glycine |
| Cmpi | 4-caboxymethyl-piperazine |
| Cpna | cyclopentyl-alanine |
| Cpra | cyclopropyl-alanine |
| Cys-NH2 | cysteinamide |
| Daa | dibasic amino acid |
| Dab | 2,4-diamino-butyric acid |
| Dab(Ac) | 2-amino-4-acetylamino-butyric acid |
| Dab(N3) | (S)-2-Amino-4-azidobutanoic acid |
| Dap | 2,4-diamino-propionic acid |
| Dip | 3,3-diphenyl-alanine |
| Disc | 1,3-dihydro-2H-isoindole-carboxylic acid |
| DLys(N3) | (R)-2-amino-6-azidohexanoic acid |
| D-NMePhe | N-methyl-D-phenylalanine |
| DPhe | D-phenylalanine |
| Dphe(4-CF3) | 4-trifluoromethyl-phenylalanine |
| Dphe(4-F) | 4-fluoro-D-phenylalanine |
| DPra | D-propargylglycine |
| Dpr(beta-Ala) | (3-aminopropionyl)-alpha,beta-diamino-propionic |
| | acid |
| DTyrMe | O-methyl-D-tyrosine |
| GAA | epsilon-guanidino-acetic acid |
| GBZA | 4-guanidino-benzoic acid |
| B-Gpa | 3-guanidino-propionic acid |
| Gly-NH2 | Glycineamide |
| GVA(Cl) | beta-chloro-epsilon-guanidino valeric acid |
| HArg | Homoarginine |
| Hcha | homocyclohexy-lalanine |
| Hcy | homo-cysteine |
| Hep | heptanoyl |
| Hgln-NH2 | Homoglutaminamide |
| hGlu | Homoglutamic acid |
| His(1-Me) | 1-methyl-histidine |
| His(3-Me) | 3-methyl-histidine |
| Hle | homo-leucine |
| Hphe | homo-phenylalanine |
| HPra | Homopropargylglycine |
| HyBA | 3,5-hydroxybenzoic acid |
| HymBA | 3-hydroxy-5-methoxybenzeneacetic acid |
| Hyp | hydroxy-proline |
| HyPA | 3,5-dihydroxyphenylacetic acid |
| Hyp(Bzl) | benzyl- hydroxy-proline |
| Igl | indanyl-glycine |
| Ile | Isoleucine |
| Inp | isonipecotic acid |
| Lys(N3) | (s)-2-amino-6-azidohexanoic acid |
| LysMe2-NH2 | N_{ω}-dimethyl-lysine amide |
| Lys-NH2 | Lysine amide |
| Lys(Z) | N-epsilon-benzyloxycarbonyl-lysine |
| Maa | 2-Mercapto-acetic acid |
| MoPA | (3-methoxyphenyl)acetic acid |
| Mpa | 3-Mercapto-propicnic acid |
| 2-Nac | 2-naphthyl-acetic acid |
| 1-Nal | 3-(1-naphthyl)-alanine |
| 2-Nal | 3-(2-naphthyl)-alanine |
| 2-Nal(N-Bzl) | N-benzyl-3-(2-naphthyl)-alanine |
| 2-Nal(N-PhEt) | N(2-phenylethyl)-3-(2-naphthyl)-alanine |
| Nba | 4-(naphthalen-2-yl)-butanoic acid |
| NDab | N-(2-aminoethyl)glycine |
| NGlu | N-(2-carboxyethyl)glycine |
| Nle | norleucine |
| NMeArg | N-methyl arginine |
| NMeDPhe | N-methyl D-phenylalanine |
| NMeHis | N-methyl histidine |
| NMe-Phe | N-methyl phenylalanine |
| NMeTrp | N-methyl tryptophan |
| Npa | 3-(naphthalen-1-yl)-propanoic acid |
| Nva | norvaline |
| (Nlys)Gly | N-(4-aminobutyl)-glycine |
| Octanoyl-PEG8-G | Octanoyl polyethylene glycol-8 glycine |
| Octanoyl-Ser | Octanoyl serine |
| Orn | ornithine |
| Orn(N3) | (S)-2-amino-5-azidopentanoic acid |
| Pal | Pyridyl-alanine |
| Pba | 4-phenyl-butanoic acid |
| Pen | penicillamine |
| Ppa | 3-phenyl-propionic acid |
| Pra | propargyl-glycine |
| Phg | phenyl-glycine |
| Phe(2-Me) | 2-methyl-phenylalanine |
| Phe(2-CN) | 2-cyano-phenylalanine |
| Phe(2-Cl) | 2-chloro-phenylalanine |
| Phe(2-F) | 2-fluoro-phenylalanine |
| Phe(2,4-Cl) | 2,4-dichloro-phenylalanine |
| D-Phe (2,4-DiCl) | 2,4-dichloro-D-phenylalanine |
| Phe(2,4-Me) | 2,4-dimethyl-phenylalanine |
| Phe(3-Me) | 3-methyl-phenylalanine |
| Phe(3-CN) | 3-cyano-phenylalanine |
| Phe(3-Cl) | 3-chloro-phenylalanine |
| Phe(3-F) | 3-fluoro-phenylalanine |
| Phe(3-Br) | 3-bromo-phenylalanine |
| Phe(3,4-diCl) | 3,4-dichloro-phenylalanine |
| Phe(3,4-diF) | 3,4-difluoro-phenylalanine |
| Phe(3,4-diOMe) | 2,4-dimethoxy-phenylalanine |
| Phe(4-Me) | 4-methyl-phenylalanine |
| Phe(4-CN) | 4-cyano-phenylalanine |
| Phe(4-Cl) | 4-chloro-phenylalanine |
| Phe(4-F) | 4-fluoro-phenylalanine |
| Phe(4-Br) | 4-bromo-phenylalanine |
| Phe(4-Me) | 4-methyl-phenylalanine |
| Phe(4-NH2) | 4-amino-Phenylalanine |
| Phe-NH2 | Phenylalanine amide |
| PheOCH3 | O-methyl-tyrosine |
| Phe(4-Ph) | 4-phenyl-phenylalanine |
| Phe(4-CF₃) | 4-trifluoromethyl-phenylalanine |
| Pip | pipecolic acid |
| Pra | Propargylglycine |
| Pra-NH2 | Propargyl-glycineamide |
| Pro-NH2 | Proline amide |
| 3-Pya | 3-pyridyl-alanine |
| PyAA | 4-pyridylacetyl acid |
| PyrProp | pyridinepropionyl group; |
| Qal(2′) | beta-(2-quinolyl)-alanine |
| R5 | R-2-(4-pentenyl)alanine |
| R8 | R-2-(7-octenyl) alanine |
| S5 | S-2-(4-pentenyl) alanine |
| S5-NH2 | S-2-(4-pentenyl) alanine amide |
| S8-NH2 | S-2-(7-octenyl) alanine amide |
| Sal | 3-styryl-alanine |
| Sar | sarcosine |
| Ser(Bzl) | O-benzyl-serine |
| Succ | succinyl |
| Thr(OBzl) | O-benzyl-threonine |
| Tic | 1,2,3,4-tetrahydroisoquinoline-3-carboxylic acid |
| Tiq | 1,2,3,4-tetrahydroisoquinoline-1-carboxytic acid |
| Thi | thienylalanine |
| Tle | tert-butylalanine |
| Tos | tosyl group |
| Tpi | 1,2,3,4-tetrahydronorharman-3-carboxylic acid |
| Trp(4-Cl) | 4-chloro-tryptophan |
| Trp(4-F) | 4-fluoro-tryptophan |
| Trp(5-Cl) | 5-chloro-tryptophan |
| Trp(5Cp) | 5-cyclopropyl-tryptophan |
| Trp(5-F) | 5-fluoro-tryptophan |
| Trp(5-Me) | 5-methyl-tryptophan |
| Trp(5-CF3) | 5-trifluoromethyl-tryptophan |
| Trp(6-Cl) | 6-chloro-tryptophan |
| Trp(6-F) | 6-fluoro-tryptophan |
| Trp(6-Me) | 6-methyl-tryptophan |
| Trp(7-Me) | 7-methyl-tryptophan |
| TrpMe | 1-methyl-tryptophan |
| Trp-NH2 | Tryptophan amide |
| Tyr(Me) | O-methyl-tyrosine |
| Tyr(Bzl) | O-benzyl-tyrosine |
| Tyr(BzlDiCl 2,6) | O-(2,6 dichloro)benzyl-tyrosine |
| Val-NH2 | Valine amide |

### Exemplary Embodiments

In some embodiments, α-MSH analogs of the present disclosure comprise a peptide sequence of the following Formula(I): Z-XAA1-XAA2-XAA3-XAA4-XAA5-XAA6-XAA7-XAA8-XAA9-XAA10-XAA11-XAA12-XAA13-Y; wherein

Z is absent, or comprises an N-terminus sequence selected from: Ac, PBA, octanoyl-PEG-GG, PyAA, HyBA, HyPA, MoPA, HymBA, C1-C10, norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, (S)-2-amino-3-(1H-indazol-3-yl)propanoic acid, Ac-Nle; Ac-Arg; 7-Ahept; BzlSO₂, PyrPropHep; 2-Nac; Nba; Npa; Pba; Ppa; Tos; or a combination thereof; optionally wherein the N-terminus sequence comprises a string of 5 or 6 amino acids (G/L-G/L-G/L-G/L-G/L-G/L), each independently selected from Glu or Lys; or optionally wherein the N-terminus sequence comprises poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., PLy-PGa-α-MSH);
wherein XAA1 is absent, comprises a Ser¹ amino acid, or comprises a moiety selected from: D-Ser, NMe-Ser, Ile, Thr, Tyr, Tyr(Me), or D-stereoisomers thereof;
wherein XAA2 is absent, comprises a Tyr² amino acid, or comprises a moiety selected from: D-Tyr, Ile, 1-NaI, 2-Nal, 2-Pal, 3-Pal, D-Phe, NMe-Phe, D-NMe-Phe, D-hPhe, Arg, D-Arg, Bip, D-Bip, Cys, D-Cys, Dip, D-Dip, His, D-1-Nal, D-2-Nal, 2-Pal, 3-Pal, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Ser, Thr, Tic, Tyr(Me), or D-stereoisomers thereof; a or Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof;
wherein XAA3 is absent, comprises a Ser³ amino acid, or comprises a moiety selected from: D-Ser, Ile, Leu, Nle, Tyr, Val, BrAc, or D-stereoisomers thereof, and optionally forms a cyclic peptide with suitable chemical groups at the 9, 10, 11 or 12 position;
wherein XAA4 is absent, comprises a Met⁴ amino acid, or comprises a moiety selected from: Lys, Lys(N3), BrAc, R4-R10, S4-S10, D-Met, Asp, Can, Cba, Cha, Cpna, Cpra, Cys, D-Cys, hCys, D-hCys, Glu, Gly, Hcy, Hle, Ile, Leu, (cyclohexyl)Gly, Nle, Nle(Met), Pen, D-Pen, Ser, Tyr, or Val, or D-stereoisomers thereof and optionally forms a cyclic peptide with suitable chemical groups at the 9, 10, 11 or 12 position;
wherein XAA5 is absent, comprises a Glu⁵ amino acid, or comprises a moiety selected from: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OrnN3, Nle, Orn, Ser, Succ, Tyr, or 4-aminobutyric acid, or D-stereoisomers thereof and optionally forms a cyclic peptide with suitable chemical groups at the 9, 10, 11 or 12 position;
wherein XAA6 is absent, comprises a His⁶ amino acid, or comprises a moiety selected from: D-His, NMe-His, NMe-D-His, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Aib, Aic, Ala, D-Ala, Arg, Asn, Asp, Cha, Chg, Cit, Cys, D-Cys, Dab, Dap, Gln, Glu, His(1-Me), His(3-Me), Hyp, Hyp(Bzl), Ile, Leu, Lys, Met, Met(O), Met(O2), Nle, D-Nle, Orn, 2-Pal, 3-Pal, 4-Pal, Phe, Pro, Sar, Ser, Ser(Bzl), Thr, Thr(OBzl), Tic, Tle, Trp, Tyr, Tyr(Me), or Val,cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, (3-furyl)alanine, or a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof and optionally forms a cyclic peptide with suitable chemical groups group at the 9, 10, 11 or 12 position;
wherein XAA7 is absent, comprises a Phe⁷ amino acid, or comprises a moiety selected from: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe),Tic, Thi, D-Thi, Trp, Tyr, D-Tyr, Tyr(Me), D-Tyr(Me), Val, or D-Val, or D-stereoisomers thereof; or a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof;
wherein XAA8 is absent, comprises a Arg⁸ amino acid, or comprises a moiety selected from: hArg, norArg, Agp, D-Arg, Arg(Me), Arg(Me)2, Ala, Cys, D-Cys, Dab, Dap, Dpr(beta-Ala), Leu, Lys, hLys, Nle, (Nlys)Gly, NMe-Arg, Orn, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4—Cl), Phe(4-CN), D-Phe(4-CN), Phe(4-F), D-Phe(4-F), Phe(4-Me), D-Phe(4-Me), Phe(4-OMe), D-Phe(4-OMe),Ser, or Trp, or D-stereoisomers thereof; or Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof;
wherein XAA9 is absent, comprises a Trp⁹ amino acid, or comprises a moiety selected from: D-Trp, Trp(6-Me), Trp(7-Me), Aic, Atc, Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq, Tpi, or D-Tpi, R4-R10, S4-S10, Ndab, Orn or D-stereoisomers thereof and optionally forms a cyclic peptide with suitable chemical groups at the 3, 4 or 5 position;
wherein XAA10 is absent, comprises a Gly¹⁰ amino acid, or comprises a moiety selected from: D-Gly, Ala, D-Ala, Arg, 6-Ahx, Cys, D-Cys, Daa, Dab, DabN3, Glu, Lys, D-Lys, hLys, LysN3, Orn, Pra, Hpra, Trp, R4-R10, S4-S10, or 5-aminopentanoic acid, or D-stereoisomers thereof and optionally forms a cyclic peptide with group at the 3, 4 or 5 position;
wherein XAA11 is absent, comprises a Lys¹¹ amino acid, or comprises a moiety selected from: D-Lys, Ala, Asn, Asp, Cys, D-Cys, Dab, Glu, hGlu, Hgin, Gly, Lys(Me)2, Pra, or Orn, or D-stereoisomers thereof and optionally forms a cyclic peptide with suitable chemical groups at the 3, 4 or 5 position;
wherein XAA12 is absent, comprises a Pro¹² amino acid, or comprises a moiety selected from: D-Pro, Ala, Asp, Cys, Gly, Ile, Leu, Met, Phe, Ser, Trp, Val, or D-stereoisomers thereof; or Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof and optionally forms a cyclic peptide with suitable chemical groups at the 3, 4 or 5 position;
wherein XAA13 is absent, comprises a Val¹³ amino acid, or comprises a moiety selected from: D-Val, NMe-Val, Ala, Can, Cba, Cha, Cpna, Cpra, Cys, Hcy, Hle, Ile, Leu, Met, Nle, Pro, or D-stereoisomers thereof; and
wherein Y is absent, or comprises a C-terminus sequence selected from: NH₂, OH, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, or Trp-NH₂.

In some embodiments, Z is Ac, PBA, octanoyl-PEG8G-G, PyAA, HyBA, HyPA, MoPA, HymBA, C7, C8, or C9; XAA1 is Ser or absent; XAA2 is Tyr or absent; XAA3 is Met or absent; XAA4 is Met or Nle or absent; XAA5 is Glu; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), Phe(4-NH2), D-Phe(4-NH2), Phe(3,4-diF), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Trp, or Ala; XAA11 is Lys, Gly, Asn, HgIn, Lys(Me)2, or absent; XAA12 is Pro or absent; XAA13 is Val or absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 35.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle or Lys(N3) or optionally Lys(N3) forms a cyclic peptide as a triazole group with Pra group at the 11 position; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 10 position; XAA11 is absent, Pra or optionally Pra forms a cyclic peptide as a triazole group with Lys(N3) group at the 4 position; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 8, SEQ ID NO: 9, SEQ ID NO: 10, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, or SEQ ID NO: 75.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; XAA11, XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 8, SEQ ID NO: 9, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, or SEQ ID NO: 75.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4—CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; XAA11 is Lys, Gly, Asn, HgIn, or Lys(Me)2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 71.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), Phe(4-NH2), D-Phe(4-NH2), Phe(3,4-diF), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; XAA11, XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 38, or SEQ ID NO: 39.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(4-NH2), D-Phe(4-NH2), Phe(3,4-diF), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; XAA11 is Lys, Gly, Asn, HgIn, or Lys(Me)2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Cys or α-Me-Cys or optionally Cys or α-Me-Cys form a cyclic peptide with Cys or α-Me-Cys group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Cys or α-Me-Cys or optionally Cys or α-Me-Cys form a cyclic peptide with Cys or α-Me-Cys group at the 5 position; XAA11 is absent, Lys, Gly, Asn, HgIn, or Lys(Me)2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequence includes SEQ ID NO: 40.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Dab or optionally Dab forms a cyclic peptide with Dab group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe (2,4-diCl), D-Phe (2,4-diCl), Phe(2,4-diMe), D-Phe(2,4-diMe), Phe(3-Cl), D-Phe(3-Cl), Phe(3-CN), D-Phe(3-CN), Phe(3-F), D-Phe(3-F), Phe(4-F), D-Phe(4-F), Phe(3-Me), D-Phe(3-Me), Phe (3,4-DiCl), D-Phe (3,4-DiCl), Phe(4-CF3), D-Phe(4-CF3), Phe(4-Cl), D-Phe(4-Cl), Phe(4-CN), Phe(4-NH2), D-Phe(4-NH2), Phe(3,4-diF), D-Phe(4-CN), Phe(4-F), D-Phe(4-F), Phe(4-Me), D-Phe(4-Me), Phe(4-OMe), D-Phe(4-OMe),Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Dab or optionally Dab forms a cyclic peptide with Dab group at the 5 position; XAA11 is absent, Lys, Gly, Asn, HgIn, or Lys(Me)2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequence includes SEQ ID NO: 41.

In some embodiments, the α-MSH analogs include specific amino acid sequences. Such amino acid sequences may include any of those listed in Table 3 or fragments or variants thereof.

TABLE 3
α-MSH analog sequences

| | | SEQ |
|---|---|---|
| ID | Sequence | ID NO: |
| KR-001 | Ac-Tyr-Ser-Met-Glu-His-Phe-Arg-Trp-Gly-Lys-Pro-Val-NH2 | 1 |
| KR-002 | Ac-Ser-Met-Glu-His-Phe-Arg-Trp-Gly-Lys-Pro-Val-NH2 | 2 |
| KR-003 | Ac-Met-Glu-His-Phe-Arg-Trp-Gly-Lys-Pro-Val-NH2 | 3 |
| KR-004 | Ac-Glu-His-Phe-Arg-Trp-Gly-Lys-Pro-Val-NH2 | 4 |
| KR-005 | Ac-Ser-Tyr-Ser-Met-Glu-His-Phe-Arg-Trp-Gly-Lys-Pro-NH2 | 5 |
| KR-006 | Ac-Ser-Tyr-Ser-Met-Glu-His-Phe-Arg-Trp-Gly-Lys-NH2 | 6 |
| KR-007 | Ac-Glu-His-Phe-Arg-Trp-Gly-Lys-Pro-NH2 | 7 |
| KR-008 | Ac-Nle-[Lys(N3)-His-Phe-Arg-Trp-Pra]-NH2 | 8 |
| KR-009 | Ac-Nle-[Lys(N3)-His-DPhe-Arg-Trp-Pra]-NH2 | 9 |
| KR-010 | Ac-[Lys(N3)-Glu-His-Phe-Arg-Trp-Gly-Pra]-NH2 | 10 |
| KR-011 | Cyclo(-Ac-Glu-His-Phe-Arg-Trp-Cys)-NH2 | 11 |
| KR-012 | Cyclo(-Ac-Glu-His-Phe-Arg-Trp-Gly-Cys)-NH2 | 12 |
| KR-013 | Ac-R5-Glu-His-Phe-Arg-Trp-Gly-S5-NH2 | 13 |
| KR-014 | Ac-S5-Glu-His-Phe-Arg-Trp-Gly-S5-NH2 | 14 |
| KR-015 | Ac-R8-Glu-His-Phe-Arg-Trp-Gly-S5-NH2 | 15 |
| KR-016 | PBA-Ser-Tyr-Ser-Met-Glu-His-Phe-Arg-Trp-Gly-Lys-Pro-Val-NH2 | 16 |
| KR-017 | Octanoyl-PEG8-G-G-Ser-Tyr-Ser-Met-Glu-His-Phe-Arg-Trp-Gly-Lys-Pro- | 17 |
| | Val-NH2 | |
| KR-018 | PyAA-Ser-Tyr-Ser-Met-Glu-His-Phe-Arg-Trp-Gly-Lys-Pro-Val-NH2 | 18 |
| KR-019 | HyBA-Ser-Tyr-Ser-Met-Glu-His-Phe-Arg-Trp-Gly-Lys-Pro-Val-NH2 | 19 |
| KR-020 | Octanoyl-Ser-Tyr-Ser-Met-Glu-His-Phe-Arg-Trp-Gly-Lys-Pro-Val-NH2 | 20 |
| KR-021 | Ac-Ser-Tyr-Ser-Met-Glu-(NMe-His)-Phe-Arg-Trp-Gly-Lys-Pro-Val-NH2 | \|21 |
| KR-022 | Ac-Ser-Tyr-Ser-Met-Glu-His-(NMe-Phe)-Arg-Trp-Gly-Lys-Pro-Val-NH2 | 22 |
| KR-023 | Ac-Ser-Tyr-Ser-Met-Glu-His-Phe-(NMe-Arg)-Trp-Gly-Lys-Pro-Val-NH2 | 23 |
| KR-024 | Ac-Ser-Tyr-Ser-Met-Glu-His-Phe-Arg-(NMe-Trp)-Gly-Lys-Pro-Val-NH2 | \|24 |
| KR-025 | Ac-Ser-Tyr-Ser-Met-Glu-His-(D-NMePhe)-Arg-Trp-Gly-Lys-Pro-Val-NH2 | 25 |
| KR-026 | Ac-Nle-R5-His-DPhe-Arg-S5-Trp-NH2 | 26 |
| KR-027 | Ac-Nle-cyc(NGlu-His-DPhe-Arg-Dab)-Trp-NH2 | 27 |
| KR-028 | Ac-Nle-cyc(Glu-His-DPhe-Arg-NDab)-Trp-NH2 | 28 |
| KR-029 | Ac-Nle-cyc(NGlu-His-DPhe-Arg-NDab)-Trp-NH2 | 29 |
| KR-030 | Ac-Nle-cyc(NDab-His-DPhe-Arg-NDab)-Trp, L: succinic acid-NH2 | 30 |
| KR-031 | Ac-Nle-cyc(aMe-Cys-His-DPhe-Arg-αMe-Cys)-Trp-NH2 | 31 |
| KR-032 | Ac-Nle-cyc(Cys-His-DPhe-Arg-Cys)-Trp, L: m-xylene-NH2 | 32 |
| KR-033 | Ac-R8-Glu-His-DPhe-Arg-Trp-Gly-S8-NH2 | 33 |
| KR-034 | Ac-Nle-R8-His-Dphe-Arg-Trp-S5-Gly-NH2 | 34 |
| KR-035 | Ac-Nle-Glu-His-Phe-Arg-Trp-Gly-Lys-NH2 | 35 |
| KR-036 | -Cyclo(-Ac-Nle-Glu-His-Phe-Arg-Trp-Gly-Lys-Cys)-NH2 | 36 |
| KR-037 | Ac-Nle-[Lys(N3)-His-DPhe-Arg-Trp-Pra]-Lys-NH2 | 37 |
| KR-038 | Ac-Nle-[R5-His-DPhe-Arg-Trp-S5]-NH2 | 38 |
| KR-039 | Ac-Nle-[S5-His-DPhe-Arg-Trp-S5]-NH2 | 39 |
| KR-040 | Ac-Nle-[Cys-His-DPhe-Arg-Trp-Cys] Linker: a,a′-Dibromo-m-xylene- | 40 |
| | NH2 | |
| KR-041 | Ac-Nle-[Dab-His-DPhe-Arg-Trp-Dab] Linker: succinic acid-NH2 | 41 |
| KR-042 | Ac-Nle-[Dab(N3)-His-DPhe-Arg-Trp-Dab(N3)] linkr: 3,3′-Oxybis[1- | 42 |
| | propyne]-NH2 | |
| KR-043 | Ac-Nle-[Lys(N3)-NMeHis-DPhe-Arg-Trp-Pra]-NH2 | 43 |
| KR-044 | Ac-Nle-[Lys(N3)-His-(NMe-Dphe)-Arg-Trp-Pra]-NH2 | 44 |
| KR-045 | Ac-Nle-[Lys(N3)-His-DPhe-NMeArg-Trp-Pra]-NH2 | 45 |
| KR-046 | Ac-Nle-[Lys(N3)-His-DPhe-Arg-NMeTrp-Pra]-NH2 | 46 |
| KR-047 | Ac-Nle-[Lys(N3)-Phe(4-NH2)-DPhe-Arg-Trp-Pra]-NH2 | 47 |
| KR-048 | Ac-Nle-[Lys(N3)-His-Dphe(4-CF3)-Arg-Trp-Pra]-NH2 | 48 |
| KR-049 | Ac-Nle-[Lys(N3)-His-Dphe(4-F)-Arg-Trp-Pra]-NH2 | 49 |
| KR-050 | Ac-Nle-[Lys(N3)-His-DPhe-NMeArg-NMeTrp-Pra]-NH2 | 50 |
| KR-051 | Ac-Nle-[Lys(N3)-NMeHis-DPhe-Arg-NMeTrp-Pra]-NH2 | 51 |
| KR-052 | Ac-Nle-[Lys(N3)-NMeHis-DPhe-NMeArg-Trp-Pra]-NH2 | 52 |
| KR-053 | HyBA-Nle-[Lys(N3)-His-DPhe-Arg-Trp-Pra]-Lys-NH2 | 53 |
| KR-054 | Ac-Nle-[Lys(N3)-His-DPhe-HArg-Trp-Pra]-Lys-NH2 | 54 |
| KR-055 | Ac-Nle-[Lys(N3)-His-DPhe-Arg(Me)2-Trp-Pra]-Lys-NH2 | 55 |
| KR-056 | Ac-Nle-[Lys(N3)-His-DPhe-Arg-Trp-Pra]-Asn-NH2 | 56 |
| KR-057 | Ac-Nle-[Lys(N3)-His-DPhe-Arg-Trp-Pra]-Hgln-NH2 | 57 |
| KR-058 | Ac-Nle-[Lys(N3)-His-DTyr(Me)-Arg-Trp-Pra]-Lys-NH2 | 58 |
| KR-059 | Ac-Nle-[Lys(N3)-His-DPhe-Arg-Trp-Pra]-Lys(Me)2-NH2 | 59 |
| KR-060 | Ac-Nle-[Lys(N3)-His-DPhe-Arg-Trp(Me)-Pra]-Lys-NH2 | 60 |
| KR-061 | Ac-Nle-[Lys(N3)-Phe(OCH3)-DPhe-Arg-Trp-Pra]-Lys-NH2 | 61 |
| KR-062 | HyPA-Nle-[Lys(N3)-His-DPhe-Arg-Trp-Pra]-Lys-NH2 | 62 |
| KR-063 | MoPA-MoPA-Nle-[Lys(N3)-His-DPhe-Arg-Trp-Pra]-Lys-NH2 | 63 |
| KR-064 | HymBA-HymBA-Nle-[Lys(N3)-His-DPhe-Arg-Trp-Pra]-Lys-NH2 | 64 |
| KR-065 | Ac-Nle-[Orn(N3)-His-DPhe-Arg-Trp-Pra]-Lys-NH2 | 65 |
| KR-066 | Ac-Nle-[Lys(N3)-His-DPhe-Arg-Trp-HPra]-Lys-NH2 | 66 |
| KR-067 | Ac-Nle-[Pra-His-DPhe-Arg-Trp-Lys(N3)]-Lys-NH2 | 67 |
| KR-068 | Ac-Nle-[Orn(N3)-His-DPhe-Arg-Trp-Hpra]-Lys-NH2 | 68 |
| KR-069 | Ac-Nle-[DLys(N3)-His-DPhe-Arg-Trp-Pra]-Lys-NH2 | 69 |
| KR-070 | Ac-Nle-[Lys(N3)-His-DPhe-Arg-Trp-DPra]-Lys-NH2 | 70 |
| KR-071 | HyBA-Nle-[Lys(N3)-His-Dphe(4-F)-Arg-Trp-Pra]-Hgln-NH2 | 71 |
| KR-072 | HyBA-Nle-[Lys(N3)-His-Dphe(4-F)-Arg-Trp-Pra]-NH2 | 72 |
| KR-073 | HyBA-Nle-[Lys(N3)-His-DPhe-Arg-Trp-Pra]-NH2 | 73 |
| KR-074 | PyAA-Nle-[Lys(N3)-His-DPhe-Arg-Trp-Pra]-NH2 | 74 |
| KR-075 | HyBA-Nle-[Lys(N3)-NMeHis-Dphe(4-F)-Arg-NMeTrp-Pra]-NH2 | 75 |
| R-1 | Ac-Ser-Tyr-Ser-Met-Glu-His-Phe-Arg-Trp-Gly-Lys-Pro-Val-NH2 | 76 |
| R-2 | Ac-Ser-Tyr-Ser-Nle-Glu-His-DPhe-Arg-Trp-Gly-Lys-Pro-Val-NH2 | 77 |
| R-3 | Ac-Ser-Tyr-Ser-Met-Glu-His-Phe-Arg-Trp-Gly-Lys-Pro-Val-Gly-Lys- | 78 |
| | Lys-Arg-Arg-Pro-Val-Lys-Val-Tyr-Pro-Asn-Gly-Ala-Glu-Asp-Glu-Ser- | |
| | Ala-Glu-Ala-Phe-Pro-Leu-Glu-Phe-NH2 | |
| R-4 | Ac-Nle-Cyc(Glu-His-Dphe-Arg-Dab)-Trp-NH2 | 79 |

In some embodiments, the α-MSH analogs comprise a peptide portion conjugated to a non-peptide-based portion. In some embodiments, the non-peptide-based portion is selected from: lipids, small molecules, RNA, DNA, polymers, or combinations thereof. In some embodiments, the α-MSH analogs comprise a peptide portion conjugated to one or more of: a cholesterol oleate moiety, a cholesteryl laurate moiety, an α-tocopherol moiety, a phytol moiety, an oleate moiety, an unsaturated cholesterol-ester moiety, or a lipophilic compound selected from acetanilides, anilides, aminoquinolines, benzhydryl compounds, benzodiazepines, benzofurans, cannabinoids, cyclic peptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids. In some embodiments, the α-MSH analogs comprise a peptide portion conjugated to one or more of: polyalkylene oxide homopolymers, polypropylene glycols, polyoxyethylenated polyols and copolymers thereof, polyethylene glycol (PEG), an albumin binding moiety, or a cell penetrating moiety. In some embodiments, the α-MSH analogs comprise a peptide portion conjugated to fatty acids, phospholipids, or sterols. In some embodiments, the α-MSH analogs comprise a peptide portion conjugated to one or more of: Palm-PEG8-G-G-Ser-Tyr (SEQ ID NO: 169); Ac-K(Palm)-G-G-Ser-Tyr (SEQ ID NO: 170), capric acid (C10), lauric acid (C12), myristic acid (C14), palmitic acid (C16), stearic acid (C18), PEG4, or PEG8.

### II. Synthesis, Formulation, and Delivery

### Synthesis

The present disclosure presents methods of synthesizing peptides and compounds of the present disclosure, including α-MSH peptide analogs and α-MSH analog compounds. In some embodiments, peptides and compounds of the present disclosure can be obtained by inducing the formation of a covalent bond between an amino group at the N-terminus of a peptide (if provided), and a carboxyl group of a reactive amino acid side chain moiety (if provided). In some embodiments, peptides and compounds of the present disclosure can be synthesizing by any known conventional procedure for the formation of a peptide linkage between amino acids. Such conventional procedures include, for example, any solution phase procedure permitting a condensation between the free alpha amino group of an amino acid or residue thereof (having its carboxyl group or other reactive groups protected) and the free primary carboxyl group of another amino acid or residue thereof (having its amino group or other reactive groups protected). In some embodiments, the peptides of the present disclosure may be synthesized by solid-phase synthesis and purified according to methods known in the art. Any of a number of well-known procedures utilizing a variety of resins and reagents may be used to prepare the peptides of the present disclosure.

In some embodiments, the process for synthesizing peptides may be carried out by a procedure whereby each amino acid in the desired sequence is added one at a time in succession to another amino acid or residue thereof. In some embodiments, the process for synthesizing peptides may be carried out by a procedure whereby multiple peptide fragments with portions of the desired amino acid sequence are first synthesized, and then condensed to provide the desired peptide sequence.

In some embodiments, the process for synthesizing peptides may be carried out using solid phase peptide synthesis, which includes methods well known and practiced in the art (e.g., Symphony Multiplex Peptide Synthesizer (Rainin Instrument Company) automated polypeptide synthesizer). In some embodiments, the process for synthesizing peptides may be carried using standard Fmoc methodology on an automated synthesizer (e.g., Advanced ChemTech 440M05, Louisville, Ky). In some embodiments, the process for synthesizing peptides may be carried using coupling reagents such as 2-(1-H-Benzotriazol-1-yl)-1,1,3,3-tetramethyluronium hexafluorophosphate (HBTU) and/or 1-Hydroxybenzotriazole (HOBt).

Solid phase peptide synthesis can be carried out by sequentially incorporating the desired amino acid residues one at a time into the growing tide chain according to the general principles of solid phase methods. These methods are disclosed in numerous references, including Merrifield, et al., Solid phase synthesis (Nobel lecture), Angew Chem (1985) 24:799-810; Barany et al., The Peptides, Analysis, Synthesis and Biology, Vol. 2; Gross et al., Eds. Academic Press 1-284 (1980), the contents of which are each incorporated herein by reference in their entirety, as related to processes and protocols for synthesizing peptides.

Solid phase synthesis of the peptide is generally commenced from the C-terminal end of the peptide by coupling a protected alpha amino acid to a suitable resin. Examples of known methods for preparing substituted amide derivatives on solid-phase have been described in the art (see, e.g., Barn D. R. et al., Tetrahedron Letters (1996), 37:3213-3216; DeGrado et al., J. Org. Chem., (1982) 47:3258-3261; the contents of which are each incorporated herein by reference in their entirety as related to methods and systems for solid-phased peptide synthesis). As an example, starting materials can be prepared by attaching an alpha amino-protected amino acid by an ester linkage to a p-benzyloxybenzyl alcohol (Wang) resin or an oxime resin by well-known means. The peptide chain is grown with the desired sequence of amino acids, and the peptide-resin is then treated with a solution of appropriate amine (such as methyl amine, dimethyl amine, ethylamine, and so on). Peptides employing a p-benzyloxybenzyl alcohol (Wang) resin may be cleaved from the resin by aluminum chloride in DCM, and peptides employing an oxime resin may be cleaved by DCM.

In some embodiments, reactive side chain groups of the various amino acid residues are protected with suitable protecting groups, which prevent a chemical reaction from occurring at that site until the protecting group is removed. In some embodiments, the alpha amino group of an amino acid residue or fragment is protected while that entity reacts at the carboxyl group, followed by the selective removal of the alpha amino protecting group to allow a subsequent reaction to take place at that site. Examples of protecting groups for use in the present disclosure have been disclosed and are known in solid phase synthesis methods and solution phase synthesis methods.

In some embodiments, alpha amino groups may be protected by a suitable protecting group, including: a urethane-type protecting group, such as benzyloxycarbonyl (Z) and substituted benzyloxycarbonyl, such as p-chlorobenzyloxycarbonyl, P-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl.

In some embodiments, guanidino amino groups (such as those found in arginine) may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc.

As one not-limiting example, solid phase synthesis of a peptide can be commenced from the C-terminal end of the peptide by coupling a protected alpha amino acid to a suitable resin. The starting material can be prepared by attaching an alpha amino-protected amino acid by an ester linkage to a p-benzyloxybenzyl alcohol (Wang) resin, a 2-chlorotrityl chloride resin or an oxime resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are then carried through repetitive addition cycles as necessary to add amino acids sequentially. The alpha amino Fmoc protecting groups are then removed under basic conditions (e.g., Piperidine, piperazine, diethylamine, or morpholine (20-40% v/v) in N,N-dimethylformamide (DMF)). Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In some embodiments, the N-terminus group is modified by introduction of an N-acetyl group. As a non-limiting example, the peptide synthesis can include a step wherein, after removal of the protecting group at the N-terminal, a resin-bound peptide is reacted with acetic anhydride in dichloromethane in the presence of an organic base, such as diisopropylethylamine. Other methods of N-terminus acetylation are known in the art, including solution phase acetylation.

In some embodiments, peptides of the present disclosure can comprise cyclic peptides having one or more bridging moieties (e.g., cyclic structure, staple, bridge, etc.).

In some embodiments, the peptide can be synthesized using solid phase peptide synthesis, and then cyclized prior to cleavage from the peptide resin. If the peptide is being cyclized through reactive side chain moieties, the desired side chains are first deprotected under specific deprotection conditions in a suitable solvent, and a cyclic coupling agent is then added. Suitable solvents include, but are not limited to: DMF, dichloromethane (DCM), and 1-methyl-2-pyrrolidone (NMP). Suitable cyclic coupling reagents include, but are not limited to: 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TPTU), and N,N′-dicyclohexylcarbodiimide/1-hydroxybenzotriazole (DCCl/HOBt). In some embodiments, coupling of the cyclic moiety to the peptide chain is initiated by use of a suitable base, such as N,N-diisopropylethylamine (DIPEA), sym-collidine, or N-methylmorpholine (NMM).

The cyclized peptides can then be cleaved from the solid phase using any suitable reagent, such as ethylamine in DCM. The resulting crude peptide is dried, and remaining amino acid side chain protecting groups (if any) are cleaved using suitable reagents, such as trifluoroacetic acid (TFA) in the presence of water and 1,2-ethanedithiol (EDT). The final product is precipitated by adding cold ether and collected by filtration. Final purification can be by reverse phase high performance liquid chromatography (RP-HPLC), using a suitable column, such as a C18 column. Other methods of separation or purification, such as methods based on the size or charge of the peptide, can also be employed. Once purified, the peptide can be characterized by any number of methods, such as high-performance liquid chromatograph (HPLC), amino acid analysis, mass spectrometry, and the like.

In some embodiments, peptides of the present disclosure can comprise one or more modifications (e.g., substitution, addition, deletion) to one or more terminus (e.g., N-terminus, C-terminus, or both) of the peptide sequence. In some embodiments, terminus-modified peptides can be synthesized using solid phase peptide synthesis, and then modified prior to cleavage from the peptide resin.

### Formulations

The present disclosure presents pharmaceutical compositions (e.g., pharmaceutical formulations) comprising α-MSH analogs of the present disclosure. In some embodiments, the present disclosure presents pharmaceutical formulations comprising α-MSH analogs of the present disclosure.

In some embodiments, the pharmaceutical formulation comprises an active ingredient (e.g., α-MSH peptide analogs) and excipients in one of the following forms: controlled release formulations, time release formulations, osmotic-controlled release delivery systems, microemulsions, microspheres, liposomes, nanoparticles, patches, pumps, drug depots, and the like. In some embodiments, the pharmaceutical formulation comprises an active ingredient (e.g., α-MSH peptide analogs) and excipients in one of the following liquid dosage forms: topical drops (e.g., eye drops), emulsions, suspensions, ointments, injections, buffered solution, isotonic solution, or aqueous solution.

In some embodiments, the pharmaceutical formulation comprises an active ingredient (e.g., α-MSH peptide analogs) and excipients in one of the following solid dosage forms: ointments, powders, gels, contacts, coatings, implants, punctal plugs, capsules, pills, and tablets.

In some embodiments, pharmaceutical compositions of the present disclosure may be administered by any route that results in a therapeutically effective outcome. Administration routes for ophthalmic indications can include: topical, eye drops (e.g., onto the conjunctiva), gels (e.g., onto the conjunctiva), and injections (e.g., subconjunctival, intravitreal). In some embodiments, the pharmaceutical composition is administered topically. In some embodiments, the pharmaceutical composition is administered topically through eye drops. In some embodiments, the pharmaceutical composition is administered topically through gel materials. In some embodiments, the pharmaceutical composition is administered by injection. In some embodiments, the pharmaceutical composition is administered by subconjunctival injection. In some embodiments, the pharmaceutical composition is administered by intravitreal injection. In some embodiments, the pharmaceutical composition is administered in an ocular implant (e.g., sustained release ocular implant). In some embodiments, the pharmaceutical composition is administered in an ocular punctual plug (e.g., sustained release punctual plug). In some embodiments, pharmaceutical compositions of the present disclosure may be administered intracamerally using a drug-eluting pellet, implant, or punctual plug.

In some embodiments, the pharmaceutical composition is administered by injection. Examples of injections include: intracameral injection (i.e., into the anterior chamber), intraocular injection, subtenon injection, retrobulbar injection, suprachoroidal injection, intracorneal injection, and intraretinal injection.

In some embodiments, α-MSH peptide analogs of the present disclosure are formulated in a sterile aqueous solution (e.g., osmolality from about 200-400 mOsm/kg and physiologically compatible pH). In some embodiments, α-MSH peptide analogs of the present invention are formulated in a lipid or non-lipid formulation. In some embodiments, α-MSH peptide analogs of the present invention are formulated in a cationic or non-cationic lipid formulation. In some embodiments, the sterile aqueous solution may contain additional active or inactive components. Inactive components, also referred to herein as “excipients” can include, but are not limited to, physiologically compatible salts, sugars, bulking agents, surfactants, or buffers.

In some embodiments, α-MSH peptide analogs of the present disclosure are formulated with or delivered in conjunction with one or more pharmaceutically acceptable excipients. In some embodiments, α-MSH peptide analogs of the present disclosure are formulated with or delivered in conjunction with one or more pharmaceutically acceptable excipients selected from solvents, buffering agents, preservatives, humectants, chelating agents, antioxidants, stabilizers, emulsifying agents, suspending agents, gel-forming agents, diluents, disintegrating agents, binding agents, lubricants, mucoadhesives, coating agents and wetting agents.

In some embodiments, α-MSH peptide analogs of the present disclosure are formulated with or delivered in conjunction with one or more pharmaceutically acceptable solvent. Examples of solvents include: water, saline solution, phosphate-buffered saline solution (PBS, alcohols, vegetable oils, marine oils, edible oils (e.g., almond oil, castor oil, cacao butter, coconut oil, corn oil, cottonseed oil, linseed oil, olive oil, palm oil, peanut oil, poppyseed oil, rapeseed oil, sesame oil, soybean oil, sunflower oil, and teaseed oil), mineral oils, fatty oils, liquid paraffin, polyethylene glycols, propylene glycols, glycerol, liquid polyalkylsiloxanes, and mixtures thereof. In some embodiments, the solvent comprises perfluorodecalin, perfluorooctane, perfluorohexyloctane, cyclomethicones (e.g., octamethylcyclotetrasiloxane, decamethylcyclopentasiloxane, dodecamethylcyclohexasiloxane), polydimethylsiloxanes, diethyl carbonate, dipropylcarbonate, or a combination thereof.

In some embodiments, α-MSH peptide analogs of the present disclosure are formulated with or delivered in conjunction with one or more pharmaceutically acceptable buffering agent. Examples of buffering agents include: citric acid, acetic acid, tartaric acid, lactic acid, hydrogenphosphoric acid, diethylamine, and mixtures thereof.

In some embodiments, α-MSH peptide analogs of the present disclosure are formulated with or delivered in conjunction with one or more pharmaceutically acceptable preservative. Examples of preservatives include: parabens, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures thereof. Examples of preservatives also include: Sofzia, purite, Xanthum Gum, or polyquad. In some embodiments, α-MSH peptide analogs of the present disclosure may be combined with one or more pharmaceutically acceptable excipient to form a pharmaceutical composition, wherein the pharmaceutical composition is substantially free of preservatives (i.e., preservative free). In some embodiments, the pharmaceutical composition comprises less than 1.0%, less than 0.1%, less than 0.01%, or less than 0.001% (w/v) of preservatives.

In some embodiments, α-MSH peptide analogs of the present disclosure are formulated with or delivered in conjunction with one or more pharmaceutically acceptable humectants. Examples of humectants include, but are not limited to, glycerin, propylene glycol, sorbitol, lactic acid, urea, and mixtures thereof.

In some embodiments, α-MSH peptide analogs of the present disclosure are formulated with or delivered in conjunction with one or more pharmaceutically acceptable chelating agents. Examples of chelating agents include: sodium EDTA and citric acid.

In some embodiments, α-MSH peptide analogs of the present disclosure are formulated with or delivered in conjunction with one or more pharmaceutically acceptable antioxidants. Examples of antioxidants include: butylated hydroxy anisole (BHA), ascorbic acid and derivatives thereof, tocopherol and derivatives thereof, cysteine, and mixtures thereof.

In some embodiments, α-MSH peptide analogs of the present disclosure are formulated with or delivered in conjunction with one or more pharmaceutically acceptable emulsifying agents. Examples of emulsifying agents include: naturally occurring gums, e.g., gum acacia or gum tragacanth; naturally occurring phosphatides, e.g., soybean lecithin; sorbitan monooleate derivatives; wool fats; wool alcohols; sorbitan esters; monoglycerides; fatty alcohols; fatty acid esters (e.g., triglycerides of fatty acids); and mixtures thereof.

In some embodiments, α-MSH peptide analogs of the present disclosure are formulated with or delivered in conjunction with one or more pharmaceutically acceptable suspending agents. Examples of suspending agents include: celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

In some embodiments, α-MSH peptide analogs of the present disclosure are formulated with or delivered in conjunction with one or more pharmaceutically acceptable gel bases and/or viscosity-increasing agents. Examples of gel bases and viscosity-increasing agents include: liquid paraffin, polyethylene, fatty oils, colloidal silica or aluminium, zinc soaps, glycerol, propylene glycol, tragacanth, carboxyvinyl polymers, magnesium-aluminium silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol aginate.

In some embodiments, α-MSH peptide analogs of the present disclosure are formulated with or delivered in conjunction with one or more pharmaceutically acceptable ointment bases. Examples of ointment bases include: beeswax, paraffin, cetanol, cetyl palmitate, vegetable oils, sorbitan esters of fatty acids (Span), polyethylene glycols, and condensation products between sorbitan esters of fatty acids and ethylene oxide, e.g., polyoxyethylene sorbitan monooleate (Tween).

In some embodiments, α-MSH peptide analogs of the present disclosure are formulated with or delivered in conjunction with one or more pharmaceutically acceptable hydrophobic ointment bases. Examples of hydrophobic ointment bases include: paraffins, vegetable oils, animal fats, synthetic glycerides, waxes, lanolin, and liquid polyalkylsiloxanes.

In some embodiments, α-MSH peptide analogs of the present disclosure are formulated with or delivered in conjunction with one or more pharmaceutically acceptable hydrophilic ointment bases. Examples of hydrophilic ointment bases include: solid macrogols (polyethylene glycols).

In some embodiments, α-MSH peptide analogs of the present disclosure are formulated with or delivered in conjunction with one or more pharmaceutically acceptable powder components. Examples of powder components include: alginate, collagen, lactose, powder which is able to form a gel when applied to a wound (absorbs liquid/wound exudate).

In some embodiments, α-MSH peptide analogs of the present disclosure are formulated with or delivered in conjunction with one or more pharmaceutically acceptable diluents and/or disintegrating agents. Examples of diluents and disintegrating agents include: lactose, saccharose, emdex, calcium phosphates, calcium carbonate, calcium sulphate, mannitol, starches and microcrystaline cellulose.

In some embodiments, α-MSH peptide analogs of the present disclosure are formulated with or delivered in conjunction with one or more pharmaceutically acceptable mucoadhesives. Examples of mucoadhesives include: thiolated acrylic acid polymers, chitosan, polyisobutyl cyanoacrylate, and ethylcellulose, Carbopol®, pectin, alginic acid, alginate, chitosan, hyaluronic acid, polysorbates, poly(ethyleneglycol), oligosaccharides and polysaccharides (e.g., Tamarind seed polysaccharide, gellan, carrageenan, xanthan gum, gum Arabic, dextran), cellulose esters and cellulose ethers, carboxymethylcellulose, hydroxyethylcellulose, hydroxypropyl methylcellulose, hydroxyethyl ethylcellulose, polyoxyethylene, poly(methyl vinyl ether), polyoxypropylene, block copolymers of ethylene oxide and propylene oxide, polyacrylamide, hydrolyzed polyacrylamide, poly(vinyl pyrrolidone), poly(methacrylic acid), poly(acrylic acid), crosslinked polyacrylic acid (e.g., Carbomer®).

In some embodiments, α-MSH peptide analogs of the present disclosure are formulated with or delivered in conjunction with one or more pharmaceutically acceptable binding agents. Examples of binding agents include: saccharose, sorbitol, gum acacia, sodium alginate, gelatine, starches, cellulose, sodium coboxymethylcellulose, methylcellulose, hydroxypropylcellulose, polyvinylpyrrolidone and polyethyleneglycol.

In some embodiments, α-MSH peptide analogs of the present disclosure are formulated with or delivered in conjunction with one or more pharmaceutically acceptable wetting agents. Examples of wetting agents include: sodium laurylsulphate and polysorbate 80.

In some embodiments, α-MSH peptide analogs of the present disclosure are formulated with or delivered in conjunction with one or more pharmaceutically acceptable lubricants. Examples of lubricants include: talcum, magnesium stearate, calcium stearate, silicium oxide, precirol and polyethylenglycol.

In some embodiments, α-MSH peptide analogs of the present disclosure are formulated with or delivered in conjunction with one or more pharmaceutically acceptable coating agent. Examples of coating agents include: hydroxypropylcellulose, hydroxypropylmethylcellulose, polyvinylpropylidone, ethylcellulose and polymethylacrylates.

In some embodiments, the α-MSH analogue may be present in the medicament in an amount of 0.001-99%, 0.01-75%, 0.1-20%, 1-15%, or 1-10% by weight of the medicament.

In some embodiments, α-MSH peptide analogs of the present disclosure are formulated with or delivered in conjunction with one or more carrier agents. Carriers are substances that aid in the delivery or improve the effectiveness of the peptides and/or peptide compositions of the present disclosure. Examples of carrier agents can include: proteins (e.g., human serum albumin (HSA)), low-density lipoproteins (LDL), globulins, carbohydrates (e.g., a dextran, pullulan, chitin, chitosan, inulin, cyclodextrin, or hyaluronic acid), lipids, synthetic polymers (e.g., synthetic polyamino acids), poly-L-lysine, poly-D-lysine, poly-L-aspartic acid, poly-D-aspartic acid, poly-L-glutamic acid, poly-D-glutamic acid, poly(L-lactide-co-glycolide) copolymer, polyethylene glycol (PEG), polyvinyl alcohol (PVA), poly(2-ethylacryllic acid), and N-isopropylacrylamide polymers. Examples of carrier agents can also include: physiological acceptable salts, poloxamer analogs with carbopol, carbopol/hydroxypropyl methyl cellulose (HPMC), carbopol-methyl cellulose, mucolytic agents (e.g., N-acetyl cysteine), carboxymethylcellulose (CMC), hyaluronic acid, cyclodextrin, and petroleum.

In some embodiments, α-MSH peptide analogs of the present disclosure may be combined with one or more pharmaceutically acceptable excipient to form a pharmaceutical composition. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol. In some embodiments, pharmaceutical compositions comprise one or more active peptide ingredients together with ethanol, corn oil-mono-di-triglycerides, hydrogenated castor oil, DL-tocopherol, propylene glycol, gelatin, glycerol, colorants, flavors, and sweeteners.

In some embodiments, α-MSH peptide analog compositions of the present disclosure can comprise or be formulated with one or more condensing agents. Condensing agents described herein may interact with (e.g., attract, hold, or bind to) peptides and act to (a) condense (e.g., reduce the size or charge of) peptides and/or (b) protect peptides (e.g., protect peptides against degradation). In some embodiments, condensing agents may include a moiety (e.g., a charged moiety), which can interact with peptides by ionic interactions. In some embodiments, condensing agents comprise charged polymers, e.g., polycationic chains. In some embodiments, condensing agents can be polylysine (PLL), spermine, spermidine, polyamine, pseudopeptide-polyamine, peptidomimetic polyamine, dendrimer polyamine, arginine, amidine, protamine, cationic lipid, cationic porphyrin, quarternary salt of a polyamine, or an alpha helical peptide.

In some embodiments, peptides of the present disclosure may be provided as prodrugs. In some embodiments wherein peptides are administered in the form of a prodrug, amino acids critical to peptide inhibitory activity are unavailable to interact with the target due to a reversible chemical bond (e.g., an ester bond). Upon administration, such prodrugs may be subject to cleavage of the reversible chemical bond, e.g., through enzymatic or acid hydrolysis in the stomach, blood and/or cells of a given target tissue.

In some embodiments, peptides of the present disclosure may be provided in the form of any pharmaceutically acceptable salt. Examples of salts derived from inorganic bases include aluminum, ammonium, calcium, copper, ferric, ferrous, lithium, magnesium, manganic salts, manganous, potassium, sodium, zinc, and the like. Examples of salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

In some embodiments where the peptide is basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts can be prepared in a suitable solvent from the peptide and an excess of an acid, such as hydrochloric, hydrobromic, sulfuric, phosphoric, acetic, trifluoroacetic, citric, tartaric, maleic, succinic or methanesulfonic acid. The acetate salt form and the ammonium acetate salt form are especially useful. Where the peptides include an acidic moiety, suitable pharmaceutically acceptable salts may include alkali metal salts, such as sodium or potassium salts, or alkaline earth metal salts, such as calcium or magnesium salts.

### III. Methods of Using the Compounds

The present disclosure presents methods and compositions for preventing and treating ophthalmic disorders. In some embodiments, α-MSH-analogs described herein can be used to treat various disorders related to ophthalmic tissues, retina, corneal epithelium and corneal MCRs. A wide range of ocular diseases and disorders may be treated by the α-MSH-analogs described herein. Non-limiting examples of ocular diseases include uveitis, glaucoma, diabetic macular edema or retinopathy, macular degeneration, corneal infection, cytomegalovirus retinitis, and dry eye (i.e., keratoconjunctivitis sicca). In some embodiments, methods of the present disclosure may be used to treat subjects suffering from ocular diseases. In some cases, methods of the present disclosure may be used to treat subjects suspected of developing ocular diseases.

In some embodiments, the present disclosure presents methods and compositions for preventing and treating disorders related to corneal endothelial cells (CEnC), including prevention of CEnC loss, and promoting CEnC survival, proliferation, and/or migration. In some embodiments, the present disclosure presents methods and compositions comprising a melanocortin receptor agonist (e.g., α-MSH peptide analogs) for use in preventing and treating disorders related to corneal endothelial cells (CEnC).

Corneal endothelial cells (CEnCs) form a monolayer on the back surface of the cornea of the eye, and are thus one of the most important structures in maintaining transparency in corneal tissue (which is likewise critical for normal vision). For example, CEnCs have been found to help control fluid and solute transport across the posterior surface of the cornea and to actively maintain the cornea in the slightly dehydrated state (which is required for optical transparency); see, e.g., Hassell et al., Exp Eye Res (2010) 91:326-35; Bourne Eye (Lond) (2003), 17:912-8.

A range of different conditions are associated with damage to CEnCs, as well as corresponding corneal swelling (e.g., edema) and reduced vision. Corneal transplantation is one common treatment for subjects with significant reduction in CEnCs. However, alternative treatments to prevent or reduce CEnC loss in various ocular and systemic conditions are still needed.

The cornea also has the highest nerve density in the body and these nerves have been shown to play an important role in maintenance of corneal cell structure and function. Little is known on how these nerves promote and regulate CEnC function, including the potential roles of melanocortin receptor agonists in regulating CEnC function.

In some embodiments, the present disclosure presents methods and compositions for preventing and treating disorders related to corneal epithelial cells. In some embodiments, the present disclosure presents methods and compositions for preventing and treating disorders related to retinal cells.

### Delivery and Treatments

In some embodiments, the present disclosure presents a method for treating or preventing CEnC diseases (e.g., CEnC loss, abnormal CEnC morphology, CEnC dysfunction) in a subject, comprising locally administering (e.g., eye drops, injection, etc.) to an eye of the subject a composition comprising an effective amount of a melanocortin receptor agonist (e.g., α-MSH peptide analog). In some embodiments, the present disclosure presents a method for treating or preventing CEnC diseases (e.g., CEnC loss) in a subject, comprising locally administering to an eye of the subject a composition comprising an effective amount of an α-MSH peptide analog of the present disclosure. In some embodiments, administration of the melanocortin receptor agonist (e.g., α-MSH peptide analog) reduces CEnC loss/dysfunction by at least 10%, 20%, 30%, 40%, 50%, 60%, 70%, 80%, 90%, 1-fold, 2-fold, 3-fold, 4-fold, 5-fold, or 10-fold, as compared to CEnC loss in an untreated eye (e.g., in an untreated control eye or subject).

In some embodiments, the present disclosure presents methods for treating or preventing one or more ophthalmic indications in a subject. In some embodiments, the present disclosure presents methods for treating or preventing one or more ophthalmic indications in a subject by administering one or more melanocortin receptor agonists (e.g., α-MSH peptide analogs) of the present disclosure to a subject. In some embodiments, the present disclosure presents methods for treating or preventing one or more ophthalmic indications in a subject by administering a therapeutically effective amount of a pharmaceutical composition to a subject, wherein the pharmaceutical composition comprises one or more melanocortin receptor agonists (e.g., α-MSH peptide analogs) of the present disclosure.

In some embodiments, the present disclosure presents methods for treating or preventing one or more ophthalmic indications in a subject, wherein the ophthalmic indication is: diabetic macular edema (DME), diabetic retinopathy (DR), cystoid macular edema (CME), retinal vein occlusion (RVO), non-infectious posterior uveitis (NIPU), glaucoma (i.e., neuroprotection in glaucoma), ocular hypertension, non-infectious uveitis, anterior uveitis, pan uveitis, Sjogrens-related dry eye, age-related macular degeneration (AMD) (including dry-AMD), Stargardt macular degeneration (SMD), or a combination thereof.

In some embodiments, the effective amount of the pharmaceutical composition is effective to increase the number of CEnCs in the subject, as compared to a subject who has not been treated with the pharmaceutical composition. In some embodiments, the effective amount of the pharmaceutical composition is effective to slow a decrease in the number of CEnCs in the subject compared to a corresponding subject who has not been treated with the pharmaceutical composition. In some embodiments, the effective amount of the pharmaceutical composition is effective to reduce apoptosis of CEnCs in the subject compared to a corresponding subject who has not been treated with the pharmaceutical composition. In some embodiments, the effective amount of the pharmaceutical composition is effective to increase proliferation of CEnCs in the subject compared to a corresponding subject who has not been treated with the pharmaceutical composition. In some embodiments, the effective amount of the pharmaceutical composition is effective to increase migration of CEnCs in the subject compared to a corresponding subject who has not been treated with the pharmaceutical composition.

In some embodiments, the pharmaceutical composition comprises a melanocortin receptor agonist (e.g., α-MSH peptide analogs) at a concentration of at least about 0.0000001 μM, 0.000001 μM, 0.00001 μM, 0.0001 μM, 0.001 μM, 0.01 μM, 0.05 μM, 0.1 μM, 0.5 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 500 μM, 600 μM, 700 μM, 800 μM, 900 μM, or 1000 μM. In some embodiments, the pharmaceutical composition comprises a melanocortin receptor agonist (e.g., α-MSH peptide analogs) at a concentration of between about 0.0000001-100 μM, 0.000001-100 μM, 0.0000001-10 μM, 0.000001-10 μM, 0.00001-0.001 μM, 0.0001-0.01 μM, 0.001-0.01 μM, 0.001-0.1 μM, 0.001-1 μM, 1-10 μM, 1-50 μM, 1-100 μM, 10-25 μM, 10-50 μM, 10-100 μM, 25-50 μM, 25-100 μM, 25-500 μM, 50-100 μM, 50-250 μM, 50-500 μM, 100-250 μM, 100-500 μM, 250-500 μM, 250-750 μM, or 500-1000 μM. In some embodiments, the pharmaceutical composition comprises a melanocortin receptor agonist (e.g., α-MSH peptide analogs) at a concentration of about 0.0000001 μM, 0.000001 μM, 0.00001 μM, 0.0001 μM, 0.001 μM, 0.01 μM, 0.05 μM, 0.1 μM, 0.5 μM or 1 μM and less than about 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 150 μM, 200 μM, 250 μM, 300 μM, 350 μM, 400 μM, 500 μM, 600 μM, 700 μM, 800 μM, 900 μM, or 1000 μM. In some embodiments, the pharmaceutical composition comprises a melanocortin receptor agonist (e.g., α-MSH peptide analogs) at a concentration of at least about 0.000000001%, 0.00000001%, 0.0000001%, 0.000001%, 0.00001%, 0.0001%, 0.001%, 0.01%, 0.1%, 1%, 5%, 10%, 20%, 30%, 40%, 50% or about 0.000000001-0.000001%, 0.000000001-0.0001%, 0.00000001-0.001%, 0.00000001-0.01%, 0.00000001-0.1%, 0.00000001-1%, 0.000001-0.00001%, 0.000001-0.0001%, 0.000001-0.001%, 0.000001-0.01%, 0.000001-0.1%, 0.000001-1%, 1-5%, 1-50%, 5-10%, 5-10%, 10-25%, 10-50%, 25-50%, or 0.000000001-50% (weight/volume). For example, a melanocortin receptor agonist may be present at concentrations of 0.000000001% (weight/volume), 0.0000001% (weight/volume), 0.00001% (weight/volume), 0.01% (weight/volume), 0.1% (weight/volume), 1% (weight/volume), 10% (weight/volume), 20% (weight/volume), 25% (weight/volume), 30% (weight/volume), 40% (weight/volume), 50% (weight/volume), or any percentage point in between.

In some embodiments, a melanocortin receptor agonist (e.g., α-MSH peptide analogs) is present in a composition or administered at a dose of at least about 0.01 μg, 0.05 μg, 0.1 μg, 0.5 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 300 μg, 350 μg, 400 μg, 500 μg, 600 μg, 700 μg, 800 μg, 900 μg, 1000 μg. In some embodiments, a melanocortin receptor agonist (e.g., α-MSH peptide analogs) is present in a composition or administered at a dose between about 0.5-100 μg, 1-10 μg, 100-1000 μg, 1-50 μg, 1-100 μg, 10-25 μg, 10-50 μg, 10-100 μg, 25-50 μg, 25-100 μg, 25-500 μg, 50-100 μg, 50-250 μg, 50-500 μg, 100-250 μg, 100-500 μg, 250-500 μg, 250-750 μg, or 500-1000 μg. In some embodiments, a melanocortin receptor agonist (e.g., α-MSH peptide analogs) is present in a composition or administered at a dose of about 0.01 μg, 0.05 μg, 0.1 μg, 0.5 μg, 1 μg, 2 μg, 3 μg, 4 μg, 5 μg, 6 μg, 7 μg, 8 μg, 9 μg, 10 g and less than about 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 300 μg, 350 μg, 400 μg, 500 μg, 600 μg, 700 μg, 800 μg, 900 μg, or 1000 μg.

In some embodiments, the pharmaceutical composition is administered to the eye of the subject on a daily basis. In some embodiments, the pharmaceutical composition is administered to the eye of the subject 2, 3, 4, 5, or 6 times per day. In some embodiments, the pharmaceutical composition is administered to the eye of the subject 1, 2, 3, 4, 5, 6, or 7 times per week.

In some embodiments, the ophthalmic indication for treatment is selected from: a corneal injury, a corneal dystrophy, an anterior corneal dystrophy, a stromal corneal dystrophy, a posterior corneal dystrophy, corneal endothelial dystrophy, Fuchs endothelial dystrophy, congenital hereditary endothelial dystrophy, posterior polymorphous corneal dystrophy, Schnyder crystalline corneal dystrophy, bullous keratopathy, neurotrophic keratophy, iridocorneal endothelial syndrome, keratitis, photokeratitis, pseudoexfoliation syndrome, ocular hypertension, glaucoma, an ocular infection, a cataract, corneal endothelial cell loss due to contact lens wear, corneal endothelial cell loss due to aging, uveitis, intraocular inflammation, inflammatory disciform keratitis, diabetes, or dry eye disease.

In some embodiments, the subject has a non-inflammatory ocular disorder. In some embodiments, the non-inflammatory ocular disorder is a non-autoimmune ocular disorder. In some embodiments, the non-autoimmune ocular disorder comprises: a corneal injury, a corneal dystrophy, an anterior corneal dystrophy, a stromal corneal dystrophy, a posterior corneal dystrophy, corneal endothelial dystrophy, Fuchs endothelial dystrophy, congenital hereditary endothelial dystrophy, posterior polymorphous corneal dystrophy, Schnyder crystalline corneal dystrophy, bullous keratopathy, iridocorneal endothelial syndrome, keratitis, neurotrophic keratopathy, ocular hypertension, glaucoma, diabetes, a cataract, an ocular infection, corneal endothelial cell loss due to contact lens wear, or corneal endothelial cell loss due to aging.

In some embodiments, the subject has a corneal dystrophy (i.e., ophthalmic disorder (genetic and progressive) in which abnormal material accumulates in the corneal layers). In some embodiments, the present disclosure presents methods for treating or preventing corneal dystrophy in a subject by administering a therapeutically effective amount of a pharmaceutical composition to a subject, wherein the pharmaceutical composition comprises one or more melanocortin receptor agonists (e.g., α-MSH peptide analogs) of the present disclosure.

In some embodiments, the subject has an anterior corneal dystrophy. In some embodiments, the subject has a stromal corneal dystrophy. In some embodiments, the subject has a posterior corneal dystrophy. In some embodiments, the subject has a corneal endothelial dystrophy. In some embodiments, the subject has a congenital hereditary endothelial dystrophy. In some embodiments, the subject has a posterior polymorphous corneal dystrophy. In some embodiments, the subject has a Schnyder crystalline corneal dystrophy. In some embodiments, the subject has a CHED1 (autosomal-dominant congenital-hereditary-endothelial-dystrophy) or CHED2 (autosomal-recessive congenital-hereditary-endothelial-dystrophy).

In some embodiments, the subject has Fuchs endothelial dystrophy. Fuchs dystrophy (also known as Fuchs' dystrophy) affects about 1% of the general population and there are no known treatments. About 100,000 corneal transplants related to Fuchs dystrophy are performed every year in the United States. In some embodiments, the subject has a variant of Fuchs endothelial dystrophy associated with a mutation in the COL8A2 gene (which encodes a protein that is part of type VIII collagen, a major component of the Descemet membrane). In some embodiments, the present disclosure presents methods for treating or preventing Fuchs endothelial dystrophy in a subject by administering a therapeutically effective amount of a pharmaceutical composition to a subject, wherein the pharmaceutical composition comprises one or more melanocortin receptor agonists (e.g., α-MSH peptide analogs) of the present disclosure.

In some embodiments, the subject will receive, is receiving, or has received ocular surgery. In some embodiments, the ocular surgery comprises intraocular surgery, cataract surgery, glaucoma surgery, cornea transplantation, intraocular lens implantation, injection of CEnCs into the eye, Descemet stripping, Descemet stripping automated endothelial keratoplasty, anterior keratoplasty, anterior lamellar keratoplasty, endothelial keratoplasty, Descemet membrane endothelial keratoplasty, Descemet stripping endothelial keratoplasty, Descemet membrane endothelial transfer, phototherapeutic keratectomy, penetrating keratoplasty, or laser eye surgery. In some embodiments, the surgery comprises vision corrective surgery. In certain embodiments, the surgery comprises laser vision corrective surgery.

In some embodiments, the subject will receive, is receiving, or has received a corneal transplant. Loss of CEnCs has been shown to be a common complication after corneal transplantation. Likewise, decreased CEnC density has been shown to be predictive of late endothelial failure, a key cause of corneal transplant failure in keratoplasty. See, e.g., Bourne et el. Cornea (2001) 20(6):560-9; Guilbert et al., Am J Ophthalmol (2013) 155(3):560-569.

In some embodiments, the melanocortin receptor agonist (e.g., α-MSH peptide analogs) as administered to the subject to reduce CEnC loss in inflammatory microenvironments (e.g., inflammatory microenvironments resulting from corneal transplantation). While grafts performed in non-vascularized host beds or low-risk grafts enjoy a success rate of approximately 90%, corneal graft rejection can exceed 50% in high-risk corneal transplantation (i.e., vascularized and/or inflamed conditions); see, e.g., Dana et al. Cornea (2000) 19(5):625-43. In some embodiments, melanocortin receptor agonists (e.g., α-MSH peptide analogs) demonstrate a protective effect on CEnCs in an inflammatory microenvironment, e.g., in subjects with both low and high risk of tissue rejection upon corneal transplantation.

In some embodiments, the melanocortin receptor agonist (e.g., α-MSH peptide analogs) as administered to the subject in combination with one or more additional ophthalmic therapeutic agents. In some embodiments, the melanocortin receptor agonist (e.g., α-MSH peptide analogs) as administered to the subject in combination with one or more additional ophthalmic therapeutic agents selected from: α-MSH, α-MSH peptide analogs, NGF, substance P, CGRP, VIP, neurotrophin-3, neurotrophin-4, neurotrophin-6, a ROCK inhibitor, or BDNF.

In some embodiments, the melanocortin receptor agonist (e.g., α-MSH peptide analogs) of the present disclosure are administered to the subject in combination with one or more stem cell therapies. In some embodiments, the melanocortin receptor agonist (e.g., α-MSH peptide analogs) of the present disclosure are administered to the subject in combination with one or more retinal stem cell therapies. Examples of retinal stem cells and retinal stem cell therapies which can be used in treatments of the present disclosure include those described in U.S. Ser. No. 10/220,117, the content of which is incorporated herein by reference in its entirety, as related to methods of producing, isolating, preparing, engineering, and using (e.g., therapeutic use with melanocortin receptor agonists) mammalian retinal stem cells. In some embodiments, the melanocortin receptor agonist (e.g., α-MSH peptide analogs) of the present disclosure are administered to the subject in combination with one or more corneal stem cell therapies (e.g., therapies using corneal endothelial cells). Examples of corneal stem cells and corneal stem cell therapies which can be used in treatments of the present disclosure include those described in US 20190119633, the content of which is incorporated herein by reference in its entirety, as related to methods of producing, isolating, preparing, engineering, and using (e.g., therapeutic use with melanocortin receptor agonists) mammalian corneal stem cells, such as human corneal endothelial cells.

In an aspect, provided herein is a cell or tissue culture medium comprising an endothelial cell and a neuropeptide. In certain embodiments, the neuropeptide comprises CGRP and/or BDNF. Also provided herein is a cell or tissue culture medium comprising an endothelial cell and a melanocortin receptor agonist (such as α-MSH or an α-MSH agonist). A cell or tissue culture medium comprising an endothelial cell and VIP is also included.

### IV. Definitions

At various places in the present disclosure, substituents, or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

ADME: As used herein, the term “ADME” refers to pharmacokinetic and pharmacological properties of a compound or formulation related to the absorption, distribution, metabolism, and excretion of the compound or formulation (i.e., the disposition and retention of a pharmaceutical compound within an organism). ADME can be used to measure and compare the delivery and tissue/cellular exposure kinetics of a formulation, as well as corresponding performance and pharmacological activity of the drug compound. Examples of tests and assays used for ADME assessment of a compound can include, but are not limited to: aqueous solubility (e.g., aqueous buffer testing), kinetic solubility, thermodynamic solubility, compound stability, protein plasma binding (e.g., equilibrium dialysis using HTD device), cell permeability (e.g., Caco-2 test), LogD distribution, LogP distribution, exposed polarity measurements (EPSA), and pK binding (e.g., in vivo pK experiments in animal tissue).

Administering: As used herein, the term “administering” refers to providing a pharmaceutical agent or composition to a subject.

Agonist: As used herein, the term “agonist” refers to a molecule or composition that can interact with a target receptor and initiate a pharmacological response characteristic of the target receptor. Accordingly, a melanocortin receptor agonist refers to a molecule or composition that can interact with a melanocortin receptor and initiate a pharmacological response characteristic of the melanocortin receptor. As used herein, the term “antagonist” refers to a molecule or composition that opposes the target receptor-associated responses normally induced by a target receptor agonist agent. Accordingly, a melanocortin receptor antagonist refers to a molecule or composition that opposes the melanocortin receptor-associated responses normally induced by a melanocortin receptor agonist agent. As used herein, the term “inverse agonist” refers to a molecule or composition that stabilizes the inactive conformation of the target receptor and inhibits basal activity. Accordingly, a melanocortin receptor inverse agonist is meant a drug or a compound that that stabilizes the inactive conformation of the melanocortin receptor and inhibits basal activity.

Amino acid: As used herein, the term “amino acids” includes the known naturally occurring protein amino acids, which are referred to by both their common three letter abbreviation and single letter abbreviation. The term “amino acid” also includes protein amino acid stereoisomers, modified protein amino acids, non-protein amino acids, post-translationally modified amino acids, enzymatically synthesized amino acids, derivatized amino acids, constructs or structures designed to mimic amino acids, and the like.

Amelioration: As used herein, the term “amelioration” or “ameliorating” refers to a lessening of severity of at least one indicator of a condition or disease. For example, in the context of ophthalmic disorders, amelioration can comprise a reduction in ophthalmic cellular degeneration, reduction in ophthalmic inflammation, improvement in ophthalmic cellular regeneration, and so forth.

Analog: As used herein, the term “analog” refers to amino acid sequence variants which differ by one or more amino acid alterations (e.g., substitutions, additions, deletions, covalent modifications) as compared to a parent or starting amino acid sequence, but still maintain one or more of the properties of the parent or starting peptide.

Approximately: As used herein, the term “approximately” or “about,” as applied to one or more values of interest, refers to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected (e.g., covalently bonded) with one another, either directly or via one or more additional moieties that serves as a linking agent, to form a structure that is sufficiently stable so that the moieties remain associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Biologically active: As used herein, the phrase “biologically active” refers to a characteristic of any substance that has activity in a biological system and/or organism. For instance, a substance that, when administered to an organism, has a biological effect on that organism, is considered to be biologically active.

Bridging moiety: As used herein, the term “bridging moiety” refers to one or more components of a cyclic peptide formed between two adjacent or non-adjacent amino acids, unnatural amino acids or non-amino acids in a peptide. Bridging moieties can also be referred to as peptide “staples”

Compound: Compounds of the present disclosure comprise all of the isotopes of the atoms occurring in the intermediate or final compounds. “Isotopes” refers to atoms having the same atomic number but different mass numbers resulting from a different number of neutrons in the nuclei. For example, isotopes of hydrogen comprise tritium and deuterium. The compounds and salts of the present disclosure can be prepared in combination with solvent or water molecules to form solvates and hydrates by routine methods.

Conjugate: As used herein, the term “conjugate” refers to any molecule or moiety appended to another molecule (e.g., a peptide conjugated to lipids, small molecules, RNA, DNA, other peptides, polymers, or combinations thereof).

Conserved: As used herein, the term “conserved” refers to nucleotides or amino acid residues of a polynucleotide sequence or peptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription, and translation of a coding sequence in a recipient cell. Not all of these control elements need always be present as long as the selected coding sequence is capable of being replicated, transcribed and/or translated in an appropriate host cell.

Conservative amino acid substitution: As used herein, the term “conservative amino acid substitution” refers to the substitution of an amino acid that is normally present in a sequence with a different amino acid of similar size, charge, or polarity. Examples of conservative substitutions include: the substitution of a non-polar (hydrophobic) residue such as isoleucine, valine and leucine for another non-polar residue; the substitution of one polar (hydrophilic) residue for another such as between arginine and lysine, between glutamine and asparagine, and between glycine and serine; the substitution of a basic residue such as lysine, arginine or histidine for another; and the substitution of one acidic residue such as aspartic acid or glutamic acid for another acidic residue.

Delivery: As used herein, “delivery” refers to the act or manner of delivering an a compound, formulation, substance, entity, or moiety.

Engineered: As used herein, embodiments of the present disclosure are “engineered” when they are designed to have a feature or property, whether structural or chemical, that varies from a starting point, wild-type or native molecule.

Effective Amount: As used herein, the terms “effective amount” and “therapeutically effective amount” of an agent is that amount sufficient to effect beneficial or desired results, for example, clinical results, and, as such, an “effective amount” depends upon the context in which it is being applied. For example, in the context of administering an agent that treats an ophthalmic indication, an effective amount of an agent is, for example, an amount sufficient to achieve treatment, as defined herein, of the ophthalmic indication, as compared to the response obtained without administration of the agent.

Feature: As used herein, a “feature” refers to a characteristic, a property, or a distinctive element.

Formulation: As used herein, a “formulation” comprises at least active compound (e.g., therapeutically active peptide) and a delivery agent or excipient.

Fragment: As used herein, the term “fragment” refers to a portion of a sequence. For example, fragments of a peptide sequence can include one or more portions of the peptide sequence, having N-terminal deletions, C-terminal deletions, internal sequence deletions, or a combination thereof.

Functional: As used herein, a “functional” biological molecule is a biological molecule in a form in which it exhibits a property and/or activity by which it is characterized.

Homology: As used herein, the term “Homology” (as it applies to amino acid sequences) refers to the percentage of residues in the amino acid sequence that are identical with the residues in a reference amino acid sequence, after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation. “Homologs” (as it applies to amino acid sequences) refer to corresponding sequence of which have substantial identity.

Identity: As used herein, the term “identity” refers to the overall relatedness between polymeric molecules, e.g., between polypeptide molecules. Calculation of the percent identity of two polypeptide sequences, for example, can be performed by aligning the two sequences for optimal comparison purposes (e.g., gaps can be introduced in one or more amino acid locations for optimal alignment). In certain embodiments, polymeric molecules can have an identity to a reference molecule at least 25%, 30%, 35%, 40%, 45%, 50%, 55%, 60%, 65%, 70%, 75%, 80%, 85%, 90%, 95%, or 99%, or similar.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. Methods for isolating compounds and their salts are routine in the art.

Mimetic: As used herein, the term “mimetic” refers to a molecule which exhibits some of the properties or features of another molecule. A “peptidomimetic” or “peptide mimetic” is a mimetic peptide in which the molecule contains structural elements that are not found in natural peptides (i.e., peptides comprised of only the 20 proteinogenic amino acids). A peptidomimetic may differ in many ways from natural polypeptides, including, but not limited to changes in structural sequence and the presence of amino acids that do not occur in nature. In some cases, peptidomimetics may include: amino acids with side chains that are not found among the known 20 proteinogenic amino acids; non-peptide-based bridging moieties used to effect cyclization between the ends or internal portions of the molecule; substitutions of the amide bond hydrogen moiety by methyl groups (N-methylation) or other alkyl groups; replacement of a peptide bond with a chemical group or bond that is resistant to chemical or enzymatic treatments; N- and C-terminal modifications; conjugation with a non-peptidic extension (such as polyethylene glycol, lipids, carbohydrates, nucleosides, nucleotides, nucleoside bases, various small molecules, or phosphate or sulfate groups); or combinations thereof.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Naturally Occurring: As used herein, “naturally occurring” or “wild-type” means existing in nature without artificial aid, or involvement of the hand of man.

Parent peptide: As used herein, the term “parent peptide” refers to the part of a peptide that does not include the terminal region (e.g., AA1-AA13 of the natural α-MSH peptide, or truncations/fragments thereof).

Peptide: As used herein, the term “peptide” refers to structure comprised of two or more amino acids, including chemical modifications and derivatives of amino adds. The term “peptide” likewise refers to a string of covalently bonded amino acids. The term “peptide” embraces “oligopeptides”, “polypeptides”, “peptidomimetics”, and “proteins”. Oligopeptides are generally considered to range in size from about 2 to about 10 amino acids. Dipeptides have two amino acid residues; tripeptides have three amino acids. Polypeptides are generally considered to range in size from about 10 to about 50 amino acids, or more. Polypeptides larger than about 50 amino acids are generally termed “proteins”.

Peptide sequences may be linear or cyclic. The amino acids forming all or a part of a peptide sequence may be naturally occurring amino acids, amino acid stereoisomers, modified amino acids, non-protein amino acids, post-translationally modified amino acids, enzymatically modified amino acids, constructs or structures designed to mimic amino adds, and the like. As such, the term “peptide” embraces pseudopeptides and peptidomimetics, including structures which have a non-peptidic backbone. The term “peptide” also includes dimers or multimers of peptides. A “manufactured” peptide includes a peptide produced by chemical synthesis, recombinant DNA technology, biochemical or enzymatic fragmentation of larger molecules, combinations of the foregoing or, in general, made by any other method.

Pharmaceutically acceptable: As used herein, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the phrase “pharmaceutically acceptable excipient” refers any ingredient other than the compounds described herein (for example, a vehicle capable of suspending or dissolving the active compound) and having the properties of being substantially nontoxic and non-inflammatory in a patient. Excipients may comprise, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients comprise, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid salts of basic residues such as amines; alkali or organic salts of acidic residues such as carboxylic acids; and the like. Representative acid addition salts comprise acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts comprise sodium, lithium, potassium, calcium, magnesium, and the like, as well as nontoxic ammonium, quaternary ammonium, and amine cations, comprising, but not limited to ammonium, tetramethylammonium, tetraethylammonium, methylamine, dimethylamine, trimethylamine, triethylamine, ethylamine, and the like.

Pharmaceutical composition: As used herein, the term “pharmaceutical composition” refers to a composition comprising at least one active ingredient (e.g., α-MSH analogs) and at least one pharmaceutically acceptable excipient. The term “pharmaceutical composition” embraces “pharmaceutical formulations”, which refers to a pharmaceutical composition comprising active ingredient and excipients in a form and amount that permits the active ingredient to be therapeutically effective.

Prodrug: As used herein, the term “prodrug” refers to a drug (e.g., peptide analog) that is provided in an inactive form that becomes active at some point after administration.

Stable: As used herein “stable” refers to a compound that is sufficiently robust to survive isolation to a useful degree of purity from a reaction mixture, and in certain embodiments, capable of formulation into an efficacious therapeutic agent. The terms “stabilize”, “stabilized,” “stabilized region” mean to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the term “synthetic” means produced, prepared, and/or manufactured by the hand of man. Synthesis of polynucleotides or polypeptides or other molecules of the present disclosure may be chemical or enzymatic.

Terminus: As used herein, the terms “termini” or “terminus”, when referring to peptides, refer to one or more extremity of a peptide. Such extremity is not limited only to the first or final moiety of the peptide but may include additional amino acids in the terminal regions. The peptide-based molecules of the present invention may be characterized as having both an N-terminus and a C-terminus.

Therapeutic Agent: As used herein, the term “therapeutic agent” refers to any agent that, when administered to a subject, has a therapeutic, diagnostic, and/or prophylactic effect and/or elicits a desired biological and/or pharmacological effect.

Treatment: As used herein, the terms “treating” and “treatment” refer to the amelioration of at least one symptom of the particular condition (even if the underlying pathophysiology of the condition is not affected). Treating a condition can include: reductions in diseased cells/tissue and/or increase in healthy cells; initiating/accelerating regression of the condition; slowing/halting/preventing progress of a condition; and/or symptomatic relief for a condition. Conditions for treatment can include injuries (e.g., trauma or a post-operative state) and disease. The efficacy of the treatment can be evaluated under various methods and conditions, including: improvement compared to a non-treatment standard; improvement in a qualitative value or parameter (e.g., self-reported pain level, vision quality, wound measurement, etc.) compared to non-treatment standard; reduction in the progression of the condition as compared to a usual time course for the condition in a non-treatment standard (e.g., historical data on progression).

Variant: As used herein, the term “variant” refers to a molecule (e.g., amino acid sequence) which differ by one or more alterations (e.g., substitutions, additions, deletions, covalent modifications) as compared to a parent or starting molecule (e.g., parent amino acid sequence). The term “derivative” can be used synonymously with the term “variant” to refer to a molecule that has been modified relative to a parent or starting molecule. The term “substitutional variant” refers to molecules (e.g., peptides) that have at least one residue (e.g., amino acid) in a native or starting sequence removed and a different residue inserted in its place at the same position. The substitutions may be single (e.g., where only one amino acid in the molecule has been substituted), or they may be multiple (e.g., where two or more amino acids have been substituted in the same molecule). The term “insertional variant” refers to molecules (e.g., peptides) that have at least one residue (e.g., amino acid) inserted immediately adjacent to an amino acid at a particular position in a native or starting sequence. The term “deletional variant” refers to molecules (e.g., peptides) that have at least one residue (e.g., amino acid) in a native or starting sequence removed from an internal region of the sequence. The term “truncated variant” refers to molecules (e.g., peptides) that have at least one residue (e.g., amino acid) in a native or starting sequence removed from the terminus of the sequence.

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1. General Synthesis of the Polypeptides

Polypeptides of the present disclosure can be synthesized by one skilled in the art using any known methods in the art. The polypeptides were synthesized by solid phase chemical peptide synthesis (SPPS) methods. The polypeptides were constructed from their individual amino acids. The amino acids can be covalently bonded to one another through functional groups, as is known in the art, where such functional groups may be present on the amino acids or introduced onto the components using one or more steps. When necessary and/or desired, certain moieties on the amino acids may be protected using blocking groups, as is known in the art, see, e.g., Green & Wuts, Protective Groups in Organic Synthesis (John Wiley & Sons) (1991).

For example, the polypeptides were synthesized using standard solid-phase Fmoc methods. The N-terminus was protected with the Fmoc group, which is stable in acid, but removable by base. Any side chain functional groups were protected with base stable, acid labile groups. The synthesis was typically performed on a peptide synthesizer using standard protocols with Rink amide resin. This resin is insoluble in the solvents used for synthesis, making it relatively simple and fast to wash away excess reagents and by-products. All amino acids were obtained from commercial sources unless otherwise noted. Coupling reagents known in the art can be used. Generally, the coupling reagent was 2-(6-chloro-1-H-benzotriazole-1yl)-1,1,3,3-tetramethylaminium hexafluorophosphate (HCTU) and the base is diisopropylethylamine (DIEA). Peptides were generally cleaved from resin with trifluoroacetic acid (TFA), water and other adducts. The crude peptides were then purified on reverse phase HPLC. Fractions containing the pure peptide were collected and lyophilized and all peptides were analyzed by LC-MS.

The linear peptides were made in a high-throughput, combinatorial fashion, e.g., using a high-throughput multi-channel combinatorial synthesizer. The peptide cyclization step was done on resin or in solution phase based on the sequences.

The peptides were further modified with, for example, N-terminal blocking agents. The N-terminus can be capped with addition of phenylbutyric acid (PBA), 3,5-dihydroxyphenylacetic acid (HyPA), (3-methoxyphenyl)acetic acid (MoPA), 3-hydroxy-5-methoxybenzeneacetic acid (HymBA), 4-pyridylacetyl acid (PyAA) etc. Such blocking agents can improve binding and confer increased corneal permeability to the peptide.

### Cyclization Procedure for SEQ ID NO: 9

The linear peptide was cleaved from the resin and washed with tert-butyl methyl ether three times and dried under vacuum for 24 hrs. Then the crude peptide (1 equiv.) was dissolved in isopropanol and water (v/v=2:1) at 1 mg per mL concentration. Copper sulfate (4 equiv., 0.315 M) and sodium ascorbate (4 equiv., 0.157 M) were dissolved in a small volume of water, mixed well, and then added into the peptide solution. The pale yellow suspension was generated, and the solution was stirred at room temperature overnight. The reaction process was monitored using RP-HPLC on peptide peaks elution time. Then EDTA (1 equiv.) was added to the reaction solution, stirring for half an hour. The reaction was then terminated.

### Cyclization Procedure for Staple Peptides—SEQ ID NO: 13, SEQ ID NO: 14, SEQ ID NO: 15, SEQ ID NO: 26, SEQ ID NO: 33, SEQ ID NO: 34

The linear peptide was synthesized through SPPS. Then the dried peptidyl resin (1 g, 0.2 mmol) was added into the round-bottom flask with 20 ML dry DCM. After shaking for 5 minutes, the Grubbs 1st catalyst (2 equiv., 0.4 mmol) was added to the reaction mixture. The reaction was shaken for three hours under Argon. A small cleavage test was done to monitor the reaction completion. After the reaction, the reaction mixture was drained and washed again with DCM and DMSO (two times each). Then the peptidyl resin was soaked in DMSO and shaken overnight. Finally, DMSO was drained and the peptidyl resin was washed again with DMSO and DCM.

### Cyclization Procedure for SEQ ID NO: 32

The linear peptide crude (1 equiv.) was dissolved in acetonitrile and water (ratio 1:2) in the concentration of 1 mg per ml. Then α,α′-Dibromo-m-xylene (1 equiv.) was added into the solution with stirring. Trimethylamine was added to adjust the reaction solution to pH 9. The reaction was monitored with LCMS and completed in one hour.

### Cyclization Procedure for SEQ ID NO: 42

The crude peptide (1 equiv.) was dissolved in isopropanol (IPA) and water (v/v=2:1) in 1 mg per mL concentration. Then 3,3′-Oxybis[1-propyne] (0.9 equiv.) was added to the peptide solution. Copper sulfate (4 equiv., 0.315 M) and sodium ascorbate (4 equiv., 0.157 M) were dissolved in a small volume of water, mixed well, and then added into the peptide solution. The pale yellow suspension was generated, and the solution was stirred at room temperature overnight. The reaction process was monitored using RP-HPLC on peptide peaks elution time. Then EDTA (1 equiv.) was added to the reaction solution, stirring for half an hour. The reaction was then terminated.

### Cyclization Procedure for SEQ ID NO: 12

The linear peptide was synthesized through SPPS. Then chloroacetic acid was the last building block which coupled at N-terminus. The linear peptide was cleaved from the resin using a TFA cocktail and washed with tert-butyl methyl ether three times. The crude peptide was dried under vacuum for 24 hrs. The linear peptide crude (1 equiv.) was dissolved in acetonitrile and water (ratio 1:2) in the concentration of 1 mg per mL. Then Trimethylamine (TEA) was added into the solution with stirring, to adjust the reaction solution to pH 9. The reaction was monitored with LCMS and completed in one hour.

### Example 2. Polypeptide Stability Assay in Water

Stock solutions of the test compounds were prepared at 10 mM in DMSO. The study was carried out in the water. Aliquots of the stock solutions were diluted to 1 mM with DMSO. Then 4 μL of the 1 mM DMSO solutions were dosed into 396 μL of water (37° C.) in tubes to reach a final test concentration of 10 μM. The tubes were kept in a 37° C. water bath for the duration of the experiment. At each time point (0, 15, 30, 60, 120, 180 minutes), 2000 μL of acetonitrile-1% formic acid (containing internal standard) was added into corresponding tubes. After the time points were quenched, the mixtures were vortexed for 1 min and centrifuged at 14000 rpm for 5 min. Aliquots of the supernatants were taken and analyzed by LC-MS/MS. The stability of SEQ ID NO: 9 was found to be 124448 h.

### Example 3. Thermodynamic Solubility Study of Polypeptides

The study was conducted in water. Water was added into compound powder to make a 10 mg/mL solution. Sample tubes were shaken at the vibrator for 1 hour (1000 rpm) and then equilibrated overnight at room temperature. Samples were centrifuged at 12000 rpm for 10 min to precipitate undissolved particles. Supernatants were transferred from tubes to a new plate. An aliquot of the supernatant after centrifugation was taken from each well, then analyzed by LC-MS/MS. The concentration of the supernatants was quantified by a standard curve. The solubility of SEQ ID NO: 9 was found to be >5 mg/mL.

### Example 4. In-Vitro Permeability Study of Polypeptides

Caco-2 cells were obtained from American Tissue Culture Collection (Rockville, MD). The cells were maintained in modified Eagle's medium (MEM), containing 10% heat-inactivated fetal bovine serum (FBS), and 1% non-essential amino acids, in CO₂ at 37° C. Cells were seeded on polycarbonate filter inserts (Millipore, CAT #PSHT 010 R5).

The cells were cultured for 21-28 days before the transport experiments. The Lucifer Yellow permeability was checked before and after the assay. Compounds were dissolved at 10 mM in 100% dimethyl sulfoxide (DMSO) and diluted for studies in Hanks Balanced Salt Solution (HBSS, Invitrogen, Cat #14025-092) with 25 mM HEPES, pH 7.4. Compounds were tested at 10 μM in both the apical-to-basolateral (A-B) and basolateral-to-apical (B-A) directions, incubations were conducted at 37° C. for 90 min. At the end of incubation, samples were diluted by assay buffer and analyzed by LC-MS/MS. The concentrations of the compounds were quantified by a standard curve. Apparent permeability coefficient (Papp) was determined to be A-B 0.77×10⁻⁶ cm·s⁻¹ B-A 0.03×10⁻⁶ cm·s⁻¹.

### Example 5. In-Vitro Assay Procedures

The cyclic AMP (cAMP) functional assay was performed on a human recombinant MC1R stable cell line (CHO-K1, GenScript). The cells were cultured at 37° C. and 5% CO₂ with culture medium (90% Ham's F-12K (Kaighn's)+10% FBS+400 μg/mL G418). The compounds and reference compound NDP-α-MSH were diluted in 4 fold starting from 50 nM. Then, 100 nL of compounds were transferred to the assay plate by the Echo machine. Cells were washed with PBS and then detached by incubation with 0.05% (w/v) Trypsin EDTA at 37° C. for 2 to 5 mins. Cells were collected with stimulation buffer (1×HBSS with 5 mM HEPES+0.2 mM IBMX+0.1% BSA) and 10 ul of cells (the density of 2,500 cells/well) solution was transferred to assay plate, centrifuged at 600 rpm for 3 min and then incubated with compounds at room temperature for 60 min. After that, 5 μL 4×Eu-cAMP tracer solution and 5 μL 4×ULight™-anti-cAMP solution was added into the cells, centrifuged at 600 rpm for 3 min and incubated for 60 min. The cAMP signal was detected with Envision® Multimode Plate Reader (PerkinElmer).

The Ca²⁺ signalling functional assay was performed on a human recombinant MC3R stable cell line (CHO-K1/Gα15, GenScript). The cells were cultured at 37° C. and 5% CO₂ with culture medium (90% Ham's F-12K (Kaighn's)+10% FBS+200 μg/mL Zeocin+100 μg/mL Hygromycin B). Cells were washed with PBS and then detached by incubation with 0.05% (w/v) Trypsin EDTA at 37° C. for 2-5 mins. Detached cells were suspended in Ham's F-12K (Kaighn's) (containing 10% FBS) and a 50 μL cell suspension was seeded into each well of a 384-well assay plate at a density of 10,000 cells/well. Cells were allowed to settle overnight at 37° C., 5% CO₂. The next day, the assay plate was centrifuged and the media replaced with dye (1×) in assay buffer (containing HBSS, HEPES 20 mM and probenecid 1.25 mM) and incubated for 1 h at 37° C. The compounds and reference compound ACTH were diluted in 4 folds starting from 50 uM. Then, 500 nL of compounds were transferred to the compound plate by the Echo machine. 30 uL assay buffer was added into the compounds plate and then shaken for 20-40 minutes. The plates were then placed on the FLIPR Tetra High-Throughput Cellular Screening System (FLIPR®, Molecular Devices) and 15 μL of the compound was added to each well, then calcium flux signal was measured.

The Ca²⁺ signalling functional assay was performed on a human recombinant MC4R stable cell line (CHO-K1/Gα15, GenScript). The cells were cultured at 37° C. and 5% CO₂ with culture medium (90% Ham's F-12K (Kaighn's)+10% FBS+8 μg/mL Puromycin+100 μg/mL Hygromycin B). Cells were washed with PBS and then detached by incubation with 0.05% (w/v) Trypsin EDTA at 37° C. for 2-5 mins. Detached cells were suspended in Ham's F-12K (Kaighn's) (containing 10% FBS) and a 50 μL cell suspension was seeded into each well of a 384-well assay plate at a density of 15,000 cells/well. Cells were allowed to settle overnight at 37° C., 5% CO₂. The next day, the assay plate was centrifuged and the media was replaced with dye (1×) in assay buffer (containing HBSS, HEPES 20 mM and probenecid 1 mM) and incubated for 1 h at 37° C. The compounds and reference compound NDP-α-MSH were diluted in 4 fold starting from 10 μM. Then, 100 nL of compounds were transferred to the compound plate by the Echo machine. 30 μL assay buffer was added to the compounds plate and then shaken for 20 to 40 minutes. The plates were placed on the FLIPR and 15 μL of the compound was added to each well, then calcium flux signal was measured.

The Ca²⁺ signalling functional assay was performed on a human recombinant MC5R stable cell line (CHO-K1/Ga15, GenScript). The cells were cultured at 37° C. and 5% C02 with culture medium (90% Ham's F-12K (Kaighn's)+10% FBS+400 μg/mL G418+100 μg/mL Hygromycin B). Cells were washed with PBS and then detached by incubation with 0.05% (w/v) Trypsin EDTA at 37° C. for 2 to 5 mins. Detached cells were suspended in Ham's F-12K (Kaighn's) (containing 10% FBS) and a 50 μL cell suspension is seeded into each well of a 384-well assay plate at a density of 15,000 cells/well. Cells were allowed to settle overnight at 37° C., 5% CO₂. The next day, the assay plate was centrifuged and the media replaced with dye (1×) in assay buffer (containing HBSS, HEPES 20 mM and probenecid 1.25 mM) and incubated for 1 h at 37° C. The compounds and reference compound NDP-α-MSH were diluted in 4 fold starting from 10 μM. Then, 100 nL of compounds were transferred to the compound plate by the Echo machine. Thirty microliter assay buffer was added to the compounds plate and then shaken for 20-40 minutes. The plates were placed on the FLIPR and 15 μL of the compound was added to each well, then calcium flux signal was measured.

IC50 values as determined by the functional assays are shown in Table 4 below.

TABLE 4
IC50 values for in-vitro assays

| | SEQ ID | MC1R | MC3R | MC4R | MC5R |
|---|---|---|---|---|---|
| ID | NO: | IC50 | IC50 | IC50 | IC50 |
| KR-001 | 1 | 0.061 | 199.3 | 87.58 | 84.38 |
| KR-002 | 2 | 0.44 | 175.5 | 34.87 | 242.82 |
| KR-003 | 3 | 0.13 | 288.7 | 40.46 | 314.9 |
| KR-004 | 4 | 1.15 | 937.2 | 34.18 | >10000 |
| KR-005 | 5 | 0.093 | 149.2 | 67.05 | 79.84 |
| KR-006 | 6 | 0.11 | 224.2 | 80.94 | 121.7 |
| KR-007 | 7 | 1.99 | 1405 | 16.39 | >10000 |
| KR-008 | 8 | 0.4 | 1109 | 74.5 | >10000 |
| KR-009 | 9 | 0.0109 | 44.1 | 45.71 | 140.4 |
| KR-010 | 10 | 170.47 | 13600.54 | 451.71 | >10000 |
| KR-011 | 11 | 127.8 | >50000 | 103.9 | Not fit |
| KR-012 | 12 | 19.98 | 5574 | 16.01 | Not fit |
| KR-015 | 15 | 25.79 | 3002.07 | 484.56 | >10000 |
| KR-016 | 16 | 0.5 | 109.26 | 62.04 | 329.09 |
| KR-017 | 17 | 0.77 | 310.13 | 139.1 | 210.19 |
| KR-018 | 18 | 0.23 | 252.7 | 94.51 | 180.44 |
| KR-019 | 19 | 0.16 | 226.5 | 151.58 | 195.14 |
| KR-020 | 20 | 0.39 | 383.26 | 154.74 | 482.85 |
| KR-021 | 21 | 0.99 | 445.37 | 82.59 | 225.1 |
| KR-022 | 22 | >200 | >50000 | 421.12 | 698.25 |
| KR-023 | 23 | 21.24 | 13379.35 | 588.64 | 4540.53 |
| KR-024 | 24 | 0.88 | 515 | 74.12 | 156.93 |
| KR-025 | 25 | 17.84 | >50000 | 182.06 | 1075.29 |
| KR-026 | 26 | >50 | >50000 | 2361.13 | 7617.86 |
| KR-027 | 27 | 0.1 | 465.03 | 84.13 | 862.74 |
| KR-028 | 28 | 0.95 | >50000 | 142.81 | 591.2 |
| KR-029 | 29 | >50 | >50000 | >1000 | >10000 |
| KR-032 | 32 | 0.081 | 1325.42 | 193.64 | 489.28 |
| KR-035 | 35 | 0.36 | 276.29 | 59.56 | 404.5 |
| KR-036 | 36 | 0.88 | 327.77 | 40.56 | 289.47 |
| KR-037 | 37 | 0.024 | 62.94 | 35.05 | 105.54 |
| KR-038 | 38 | >50 | 19952.16 | 73.97 | 5092.3 |
| KR-039 | 39 | 1.95 | 426.25 | 35.39 | 738.93 |
| KR-040 | 40 | 0.124 | 143.08 | 109.89 | 256.82 |
| KR-041 | 41 | 0.04 | 24.11 | 13.76 | 44.26 |
| KR-042 | 42 | 0.16 | 224.79 | 108.94 | 237.39 |
| KR-043 | 43 | 0.15 | 61.57 | 31.38 | 85.99 |
| KR-044 | 44 | >50 | >50000 | 237.97 | 2303.88 |
| KR-045 | 45 | 0.48 | >50000 | 40.32 | 190.81 |
| KR-046 | 46 | 0.28 | 98.79 | 39.28 | 94.8 |
| KR-047 | 47 | >50 | 80.91 | 17.89 | 41.92 |
| KR-048 | 48 | 0.18 | >50000 | 183.02 | 48.81 |
| KR-049 | 49 | 0.019 | 21.3 | 16.11 | 25.19 |
| KR-050 | 50 | 0.081 | 4482.35 | 241.72 | 234.34 |
| KR-051 | 51 | 0.043 | 77.51 | 96.19 | 98.87 |
| KR-052 | 52 | 2.68 | >50000 | 121.69 | 194.71 |
| KR-053 | 53 | 0.0089 | 78.11 | 39.8 | 119.77 |
| KR-054 | 54 | 0.093 | 67.82 | 31.86 | 82.34 |
| KR-056 | 56 | 0.052 | 66.47 | 23.07 | 69.56 |
| KR-057 | 57 | 0.024 | 73.33 | 33.51 | 107.09 |
| KR-058 | 58 | 2.68 | >50000 | 121.69 | 194.71 |
| KR-060 | 60 | 0.79 | 260.35 | 67.59 | 319.03 |
| KR-061 | 61 | 28.04 | 51.08 | 101.32 | 149.98 |
| KR-062 | 62 | 0.03 | 13 | 44.37 | 166.78 |
| KR-063 | 63 | 0.05 | 62.79 | 67.11 | 99.6 |
| KR-065 | 65 | 0.027 | 91.6 | 35.95 | 70.22 |
| KR-066 | 66 | 0.0047 | 39.86 | 75.07 | 63.96 |
| KR-067 | 67 | 0.11 | 66.23 | 28.69 | 79.73 |
| KR-068 | 68 | 0.04 | 27.65 | 21.8 | 40.35 |
| KR-069 | 69 | 16.49 | 142.87 | 30.85 | 160.71 |
| KR-070 | 70 | 11.81 | 1631.87 | 84.14 | 375.84 |
| KR-071 | 71 | 0.0023 | 49.01 | 103.41 | 73.93 |
| KR-072 | 72 | 0.0019 | 43.47 | 98.57 | 78.05 |
| KR-073 | 73 | 0.0041 | 126.55 | 13.89 | 133.66 |
| KR-074 | 74 | 0.023 | 165.14 | 20.21 | 153.63 |

### Example 6. In Vivo Administration Study—Rabbit

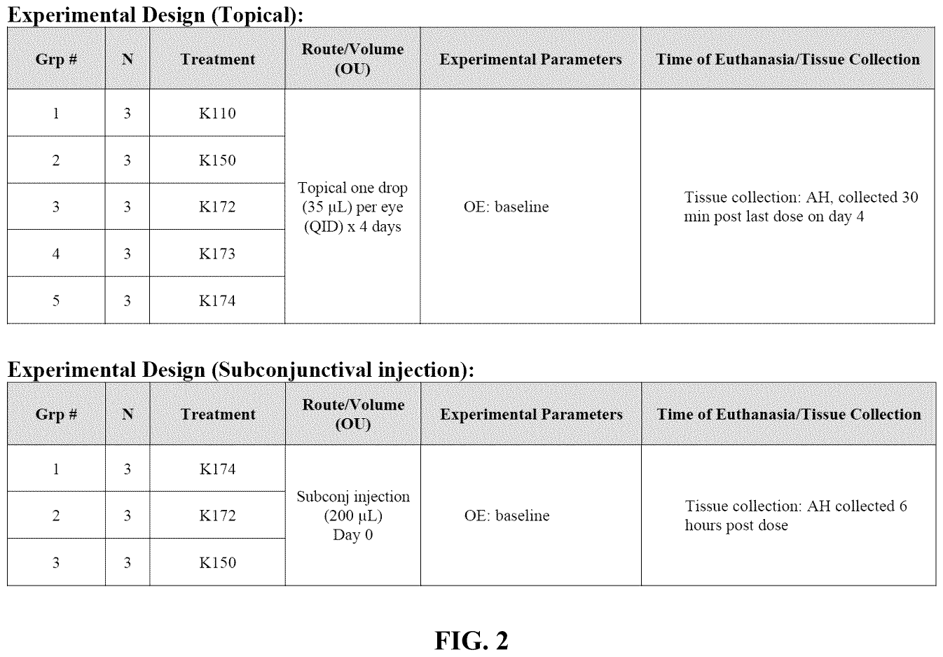

To evaluate the effects of the peptide formulations following topical administration and subconjunctival injection a study in rabbits was undertaken. Animals were acclimated to the study and, upon completion of the acclimation period, each animal was physically examined for determination of suitability for study participation. Animals determined to be in good health were randomized into study groups to achieve averaged mean body weight in each group. (N=24; 3 animals per study group) Animals will be uniquely identified by corresponding cage card number and ear tag. Tables summarizing the parameters regarding the study are provided in FIG. 2.

The K110 (KR-009; SEQ ID NO: 9) peptide was formulated in a 3 mg/ml sterile solution with PBS and the K150 (KR-049; SEQ ID NO: 49), K172 (KR-071; SEQ ID NO: 71), K173 (KR-072; SEQ ID NO: 72), and K174 (KR-073; SEQ ID NO: 73) peptides were formulated in a 3 mg/ml sterile solution in 5% Mannitol in sterile water solution. For topical ocular dosing the designated peptide formulations (35 L) were administered to the eye of each animal using a calibrated pipette. With the animal manually restrained, the upper eyelid of the eye was gently elevated to expose the cornea. Treatment was then be applied to the cornea without contacting the eye with the pipette tip. One topical drop was applied per eye for four days. Tissue collection (aqueous humor) occurred 30 minutes after last dose.

For subconjunctival injection on the day of injection animals are given an injection of buprenorphine (0.01-0.05 mg/kg SC) and anesthetized with an intramuscular injection of ketamine hydrochloride (50-80 mg/kg) and xylazine hydrochloride (5-10 mg/kg). Aseptic precautions are taken for the injections. Following induction of anesthesia, 5% betadine solution, on autoclaved sterile gauze, was used to clean the periocular (eyelids) area of the eye. Betadine was used to irrigate the ocular surface and conjunctival cul-de-sacs of the eye. Sterile eyewash was used to thoroughly irrigate the ocular surface of the eye. One drop each of 0.5% proparacaine HCL and 10% phenylephrine HCL were applied to the ocular surface of the eye. The conjunctiva was gently grasped with Colibri forceps and using a 27-30 gauge needle the solution (200 μL) was delivered inferiorly. Rabbits were given a topical antibiotic (Neo-Poly Gramicidin) following the injection and returned to their cage to recover. Tissue collection (aqueous humor) occurred 6 hours after last dose.

A veterinary ophthalmologist performed complete ocular examinations using a slit lamp biomicroscope and indirect ophthalmoscope to evaluate ocular surface morphology and anterior and posterior segment inflammation on all animals prior to injection to serve as a baseline for enrollment into the study as well as on specific study days. Animals were not tranquilized for the examinations.

At least 1.0 mL of whole blood was drawn from the marginal ear vein or cardiac puncture (terminal bleed only) into K₂EDTA tubes for plasma collection. After collection, the tubes were gently mixed by inverting the tubes 5-8 times. Blood samples were stored on wet ice prior to plasma processing. The samples were centrifuged at 4° C. for 10 minutes at 2000 g in a swinging bucket refrigerated centrifuge. Within 20 minutes of the blood collection time, the clear plasma was transferred to prelabelled polypropylene tubes, snap frozen, and stored frozen at approximately −80° C.

Following euthanasia, eyes were enucleated and marked at the 12:00 position. Aqueous humor was removed via a 27 or 30 gauge syringe following a 10-15 second rinse with PBS and blot with Kimwipe, and snap frozen by immersing in liquid nitrogen. The whole globe was then snap frozen in liquid nitrogen and stored at −80° C. to be held for potential dissection. All samples were placed into individual vials and weighed. The plasma and aqueous humor were collected.

The peptides were evaluated for their ocular tolerability and pharmacokinetics following repeated (QID×4 days) topical instillation or a single subconjunctival injection in rabbits. The study examined penetration into the anterior chamber and concentrations in the plasma by measuring at a single time point. The measured concentrations of K110, K150, K172, K173, and K174 in the aqueous humor of the rabbit are provided in Table 5. The data demonstrated that higher aqueous humor concentrations were achieved with subconjunctival administration.

TABLE 5
Concentrations of K110, K150, K172, K173,
and K174 in Rabbit Aqueous Humor

| Group 1 | K110 | Group 2 | K150 |
|---|---|---|---|
| Topical | Concentrations | Topical | Concentrations |
| Timepoint | (ng/mL) in | Timepoint | (ng/mL) in |
| D 4 30 Min | Rabbit Aqueous | D 4 30 Min | Rabbit Aqueous |
| Sample ID | Humor | Sample ID | Humor |
| 10452-OS | BQL (2.00) | 10455-OS | 55.4 |
| 10452-OD | BQL (2.00) | 10455-OD | 67.7 |
| 10453-OS | 7.06 | 10456-OS | 3.83 |
| 10453-OD | 2.88 | 10456-OD | 3.98 |
| 10454-OS | BQL (2.00) | 10457-OS | 14.7 |
| 10454-OD | BQL (2.00) | 10457-OD | 7.37 |
| Group 3 | K172 | Group 4 | K173 |
| Topical | Concentrations | Topical | Concentrations |
| Timepoint | (ng/mL) in | Timepoint | (ng/mL) in |
| D 4 30 Min | Rabbit Aqueous | D 4 30 Min | Rabbit Aqueous |
| Sample ID | Humor | Sample ID | Humor |
| 10458-OS | 2.84 | 10462-OS | 15.3 |
| 10458-OD | 2.35 | 10462-OD | 21.9 |
| 10459-OS | 3.42 | 10463-OS | 11.1 |
| 10459-OD | 2.80 | 10463-OD | 31.1 |
| 10460-OS | 3.66 | 10464-OS | 14.7 |
| 10460-OD | 4.15 | 10464-OD | 14.5 |

| Group 5 | K174 | Group 1 | K174 |
|---|---|---|---|
| Topical | Concentrations | SubConj. Inj. | Concentrations |
| Timepoint | (ng/mL) in | Timepoint | (ng/mL) in |
| D 4 30 Min | Rabbit Aqueous | D 0 6 hr | Rabbit Aqueous |
| Sample ID | Humor | Sample ID | Humor |
| 10465-OS | 7.91 | 10468-OS | 54.1 |
| 10465-OD | 11.4 | 10468-OD | 90.3 |
| 10466-OS | 9.51 | 10469-OS | 101 |
| 10466-OD | 8.18 | 10469-OD | 124 |
| 10467-OS | 11.7 | 10470-OS | 46.4 |
| 10467-OD | 35.8 | 10470-OD | 173 |

| Group 2 | K172 | Group 3 | K150 |
|---|---|---|---|
| SubConj. Inj. | Concentrations | SubConj. Inj. | Concentrations |
| Timepoint | (ng/mL) in | Timepoint | (ng/mL) in |
| D 0 6 hr | Rabbit Aqueous | D 0 6 hr | Rabbit Aqueous |
| Sample ID | Humor | Sample ID | Humor |
| 10471-OS | 47.9 | 10474-OS | 47.8 |
| 10471-OD | 54.4 | 10474-OD | 129 |
| 10472-OS | 50.9 | 10475-OS | 106 |
| 10472-OD | 98.7 | 10475-OD | 227 |
| 10473-OS | 66.4 | 10476-OS | 81.2 |
| 10473-OD | 128 | 10476-OD | 95.3 |

*BQL (Conc.) = Below Quantitation Limit (ng/mL)

### Example 7. In Vivo Topical Administration Study—Rabbit

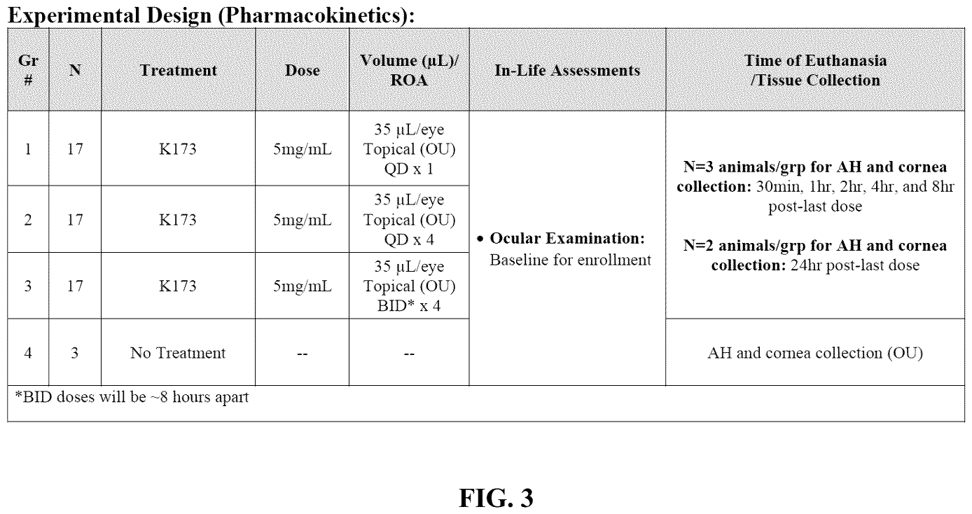

To evaluate the pharmacokinetics of the peptide formulations following topical administration a study in rabbits was undertaken. Animals were acclimated to the study and, upon completion of the acclimation period, each animal was physically examined for determination of suitability for study participation. Animals determined to be in good health were randomized into study groups (N=51; 17 animals per study group and 3 animals per control group). Animals will be uniquely identified by corresponding cage card number and ear tag. A Table summarizing the parameters regarding the study is provided in FIG. 3.

The K173 (KR-072; SEQ ID NO: 72) peptide was formulated in a 5 mg/ml sterile solution with HPMC, histidine buffer, and mannitol. The designated peptide formulations (35 L) were administered to the eye of each animal using a calibrated pipette. With the animal manually restrained, the upper eyelid of the eye was gently elevated to expose the cornea. Treatment was then be applied to the cornea without contacting the eye with the pipette tip. The animal was then allowed to blink several times while still manually restrained to distribute the applied solution over the eye prior to being returned to the cage. Treatment was administered once (QD) or twice daily (BID) with approximately 8 hours between BID doses. Group 1 was administered a single dose, Group 2 received 4 doses, Group 3 received 8 BID doses. Samples were collected from 3 animals/group 30 min, 1 hr, 2 hr, 4 hr, and 8 hr post-last dose and from 2 animals/group 24 hours post-last dose.

A veterinary ophthalmologist performed complete ocular examinations using a slit lamp biomicroscope and indirect ophthalmoscope to evaluate ocular surface morphology and anterior and posterior segment inflammation on all animals prior to injection to serve as a baseline for enrollment into the study as well as on specific study days. Animals were not tranquilized for the examinations.

Following euthanasia, eyes were enucleated, rinsed 2× with fresh ice-cold PBS, and dabbed dry with gauze. Aqueous humor was removed via a 27- or 30-gauge syringe, placed into a pre-weighted 2 mL polypropylene tube, reweighed, and snap frozen by immersion in liquid nitrogen. The cornea was harvested fresh from each eye, placed into a pre-weighted re-enforced 2 mL polypropylene tube, re-weighed and snap frozen in liquid nitrogen. The remaining ocular tissues were discarded. Ocular samples were stored −80° C.

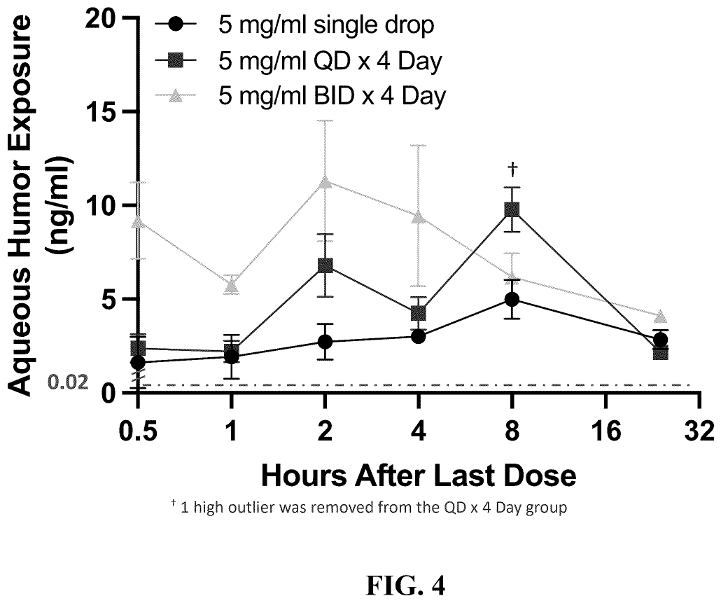

As shown in FIG. 4, K173 was shown to be delivered to the target tissue. K173 was shown to have prolonged ocular surface retention and Tₘₐₓ after a single dose. Aqueous humor (AH) concentrations were shown to increase with repeated dosing. In fact, measurable levels were detected after 24 hours in all dose groups. The concentrations of K173 in the aqueous humor was measured for each group at different time points and the values are provided in Table 6. The rabbit efficacy model can be used to determine if QD or BID dosing is optimal for clinical utility.

TABLE 6
Concentration of K173 in Rabbit Aqueous Humor

| | K173 | K173 | | K173 | K173 |
|---|---|---|---|---|---|
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 30 Min | Rabbit Aqueous | Rabbit Aqueous | 30 Min | Rabbit Aqueous | Rabbit Aqueous |
| Animal | Humor | Humor | Animal | Humor | Humor |
| ID | OS Sample | OD Sample | ID | OS Sample | OD Sample |
| 11407 | BQL (1.00) | 8.44 | 11538 | 3.87 | 1.90 |
| 11408 | 1.36 | BQL(1.00) | 11539 | 1.30 | BQL(1.00) |
| 11409 | BQL(1.00) | BQL(1.00) | 11540 | 5.23 | 1.90 |
| Mean | 1.36 | 8.44 | Mean | 3.47 | 1.90 |
| SD | NA | NA | SD | 1.995 | 0.00375 |
| % CV | NA | NA | % CV | 57.5% | 0.2% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 1 hr | Rabbit Aqueous | Rabbit Aqueous | 1 hr | Rabbit Aqueous | Rabbit Aqueous |
| Animal | Humor | Humor | Animal | Humor | Humor |
| ID | OS Sample | OD Sample | ID | OS Sample | OD Sample |
| 11410 | BQL(1.00) | 1.13 | 11541 | BQL(1.00) | 1.60 |
| 11525 | 7.15 | BQL(1.00) | 11542 | 3.37 | 3.74 |
| 11526 | 3.30 | BQL(1.00) | 11543 | 1.62 | 2.90 |
| Mean | 5.22 | 1.13 | Mean | 2.49 | 2.75 |
| SD | 2.73 | NA | SD | 1.24 | 1.07 |
| % CV | 52.2% | NA | % CV | 49.8% | 39.1% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 2 hr | Rabbit Aqueous | Rabbit Aqueous | 2 hr | Rabbit Aqueous | Rabbit Aqueous |
| Animal | Humor | Humor | Animal | Humor | Humor |
| ID | OS Sample | OD Sample | ID | OS Sample | OD Sample |
| 11527 | 2.90 | 7.40 | 11544 | 11.0 | 9.23 |
| 11528 | 1.46 | 1.38 | 11545 | 10.7 | 5.64 |
| 11529 | 1.30 | 1.95 | 11546 | 1.80 | 2.40 |
| Mean | 1.89 | 3.58 | Mean | 7.84 | 5.76 |
| SD | 0.882 | 3.33 | SD | 5.23 | 3.41 |
| % CV | 46.7% | 93.0% | % CV | 66.7% | 59.3% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 4 hr | Rabbit Aqueous | Rabbit Aqueous | 4 hr | Rabbit Aqueous | Rabbit Aqueous |
| Animal | Humor | Humor | Animal | Humor | Humor |
| ID | OS Sample | OD Sample | ID | OS Sample | OD Sample |
| 11530 | 3.56 | 2.94 | 11547 | 6.69 | 4.61 |
| 11531 | 4.04 | 2.79 | 11548 | 2.48 | 6.84 |
| 11532 | 2.46 | 2.31 | 11549 | 2.41 | 2.39 |
| Mean | 3.35 | 2.68 | Mean | 3.86 | 4.61 |
| SD | 0.811 | 0.329 | SD | 2.45 | 2.23 |
| % CV | 24.2% | 12.3% | % CV | 63.5% | 48.3% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 8 hr | Rabbit Aqueous | Rabbit Aqueous | 8 hr | Rabbit Aqueous | Rabbit Aqueous |
| Animal | Humor | Humor | Animal | Humor | Humor |
| ID | OS Sample | OD Sample | ID | OS Sample | OD Sample |
| 11404 | 3.34 | 3.90 | 11535 | 6.41 | 11.3 |
| 11405 | 5.09 | 3.40 | 11536 | 9.88 | 8.07 |
| 11406 | 4.17 | 10.0 | 11537 | 28.1 | 13.2 |
| Mean | 4.20 | 5.77 | Mean | 14.8 | 10.9 |
| SD | 0.875 | 3.68 | SD | 11.6 | 2.62 |
| % CV | 20.8% | 63.8% | % CV | 78.7% | 24.1% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 24 hr | Rabbit Aqueous | Rabbit Aqueous | 24 hr | Rabbit Aqueous | Rabbit Aqueous |
| Animal | Humor | Humor | Animal | Humor | Humor |
| ID | OS Sample | OD Sample | ID | OS Sample | OD Sample |
| 11533 | 2.34 | 4.32 | 11575 | 2.30 | 2.34 |
| 11534 | 2.58 | 2.11 | 11576 | 2.36 | 1.61 |
| Mean | 2.46 | 3.22 | Mean | 2.33 | 1.98 |
| SD | 0.175 | 1.56 | SD | 0.0431 | 0.518 |
| % CV | 7.1% | 48.4% | % CV | 1.8% | 26.2% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 30 Min | Rabbit Aqueous | Rabbit Aqueous | 1 hr | Rabbit Aqueous | Rabbit Aqueous |
| Animal | Humor | Humor | Animal | Humor | Humor |
| ID | OS Sample | OD Sample | ID | OS Sample | OD Sample |
| 11582 | 8.54 | 9.70 | 11585 | 7.48 | 5.13 |
| 11583 | 12.7 | 16.5 | 11586 | 6.40 | 6.00 |
| 11584 | 3.10 | 4.63 | 11587 | 5.80 | 3.86 |
| Mean | 8.11 | 10.3 | Mean | 6.56 | 5.00 |
| SD | 4.80 | 5.94 | SD | 0.855 | 1.08 |
| % CV | 59.3% | 57.8% | % CV | 13.0% | 21.5% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 2 hr | Rabbit Aqueous | Rabbit Aqueous | 4 hr | Rabbit Aqueous | Rabbit Aqueous |
| Animal | Humor | Humor | Animal | Humor | Humor |
| ID | OS Sample | OD Sample | ID | OS Sample | OD Sample |
| 11588 | 10.8 | 9.68 | 11591 | 6.53 | 4.11 |
| 11589 | 22.5 | 18.5 | 11592 | 2.82 | 3.82 |
| 11590 | 3.11 | 3.28 | 11593 | 12.6 | 26.8 |
| Mean | 12.1 | 10.5 | Mean | 7.33 | 11.6 |
| SD | 9.76 | 7.62 | SD | 4.96 | 13.2 |
| % CV | 80.5% | 72.8% | % CV | 67.7% | 113.9% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 8 hr | Rabbit Aqueous | Rabbit Aqueous | 24 hr | Rabbit Aqueous | Rabbit Aqueous |
| Animal | Humor | Humor | Animal | Humor | Humor |
| ID | OS Sample | OD Sample | ID | OS Sample | OD Sample |
| 11578 | 9.10 | 3.65 | 11594 | 3.94 | 3.34 |
| 11580 | 4.24 | 3.33 | 11595 | 4.96 | 4.24 |
| 11581 | 5.93 | 10.8 | Mean | 4.45 | 3.79 |
| Mean | 6.42 | 5.92 | SD | 0.717 | 0.639 |
| SD | 2.46 | 4.21 | % CV | 16.1% | 16.9% |
| % CV | 38.4% | 71.2% | | | |

*BQL (Conc.) = Below Quantitation Limit (ng/mL); NA: Not Applicable

### Example 8. In Vivo Topical Administration Study—Rabbit

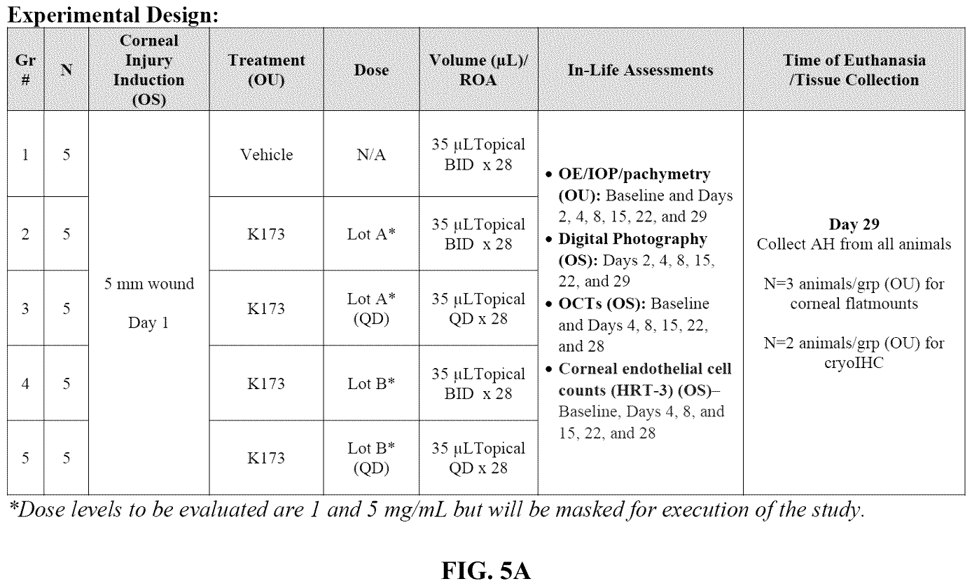

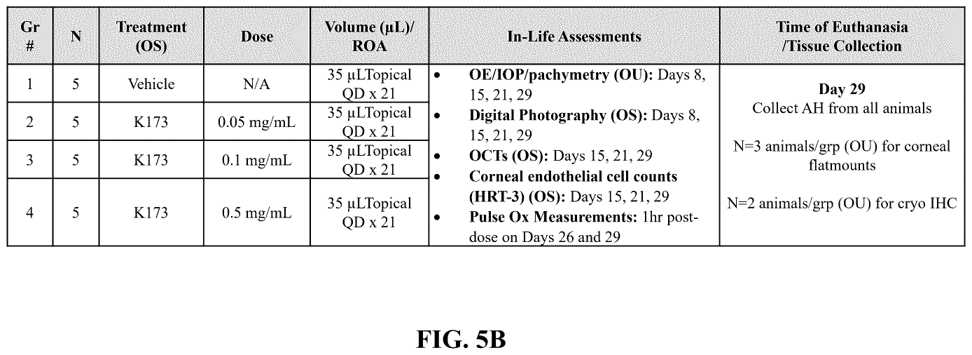

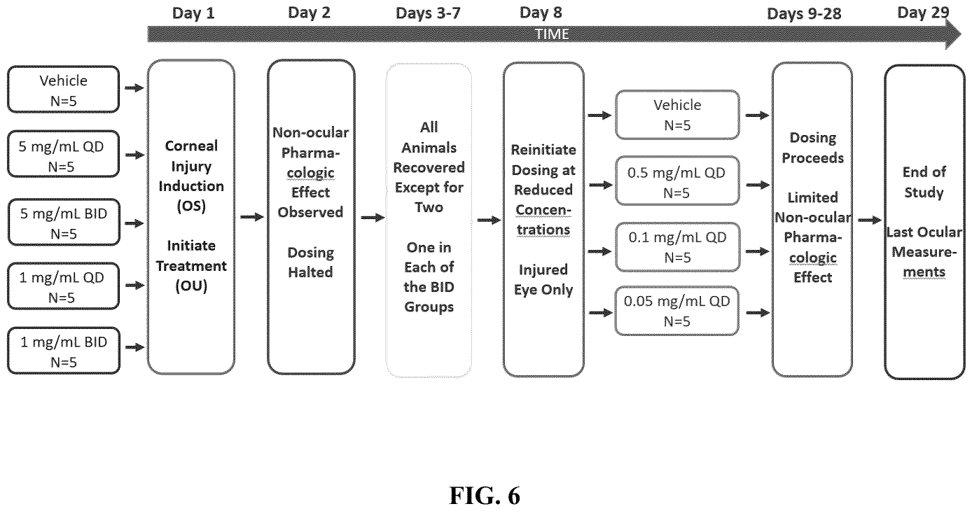

To evaluate the efficacy of a peptide formulation in reducing corneal edema following Descemetorhexis without Endothethelial Keratoplasty (DWEK) surgery a study in rabbits was undertaken. Animals were acclimated to the study and, upon completion of the acclimation period, each animal was physically examined for determination of suitability for study participation. Animals determined to be in good health were randomized into study groups (N=25; 5 animals per study group). Animals will be uniquely identified by corresponding cage card number and ear tag. A Table summarizing the parameters regarding the study is provided in FIGS. 5A-5B and a schematic exemplifying the timeline of the study is provided in FIG. 6.

The K173 (KR-072; SEQ ID NO: 72) peptide was formulated in a 1 mg/ml or 5 mg/ml sterile solution with HPMC, histidine buffer, and mannitol. The designated peptide formulations (35 μL) were administered to the eye of each animal using a calibrated pipette. With the animal manually restrained, the upper eyelid of the eye was gently elevated to expose the cornea. Treatment was then applied to the cornea without contacting the eye with the pipette tip. The animal was then allowed to blink several times while still manually restrained to distribute the applied solution over the eye prior to being returned to the cage. Treatment was administered once (QD) or twice daily (BID) with approximately 6-8 hours between each BID dose for 28 days.

The rabbits were subjected to a surgical procedure prior to administering topical treatment. The animals received buprenorphine (0.01-0.05 mg/kg SC) and were sedated immediately prior to the procedure with ketamine/xylazine cocktail (20-50/4-10 mg/kg IM). Aseptic precautions were taken for the surgical procedure. Following induction of anesthesia, 5% betadine solution was used to clean the periocular (eyelids) area of the left eye. Betadine was then used to irrigate the ocular surface and conjunctival cul-de-sacs of the left eye. Sterile eyewash was then used to irrigate the ocular surface of the left eye. One drop each of 0.5% proparacaine HCL and 10% phenylephrine HCL was applied to the ocular surface of the left eye. The animal's corneal epithelium was marked with a circular ring (5 mm) to create a template for resection of the host tissue. A 2.4 mm keratome was used to create a superior incision. Descemet's membrane was scored peripherally using a reverse Terry-Sinskey hook, and then Descemet's membrane was peeled from the overlying stroma. Descemet's membrane was removed using forceps. Viscoelastic was removed by Irrigation Aspiration (IA) using balanced salt saline (BSS). One (1) ml of epinephrine 1:1000 and 0.5 ml of heparin (10,000 USP units/ml) was added to each 500 ml of irrigation solution (BSS) to facilitate pupil dilation, control inflammation, and reduce fibrin formation. Following closure with 9-0 nylon suture, BSS was injected to normalize the intraocular pressure. The rabbits were given a topical antibiotic (Neo-Poly Gramicidin) following surgery followed by topical Prednisolone Acetate (Prednisolone Acetate ophthalmic suspension, 1.0%), approximately 10 min after the antibiotic drop. The first dose of the test article was administered approximately 10 min after the Prednisolone dose. 4-6 hours after surgery, the rabbits were given another drop of Prednisolone Acetate and then BID for 7 days following surgery (AM and PM). The animals were also given topical antibiotics BID for 3 days following surgery. At least 5 minutes were allowed between Prednisolone Acetate and antibiotic drops. The test article (i.e., K173 formulation) was administered after the Prednisolone and antibiotic drops with at least 5 minutes separating the doses. Animals were given an additional dose of buprenorphine 4-6 hours after completion of the surgery.

After commencement of the study and initial administration of the test articles, certain non-ocular pharmacologic effects were observed and dosing was halted. After the animals recovered dosing was reinitiated at reduced concentrations of 0.5 mg/mL QD, 0.1 mg/mL QD, and 0.05 mg/mL QD and the study proceeded (FIG. 5B).

A veterinary ophthalmologist performed complete ocular examinations (OE) using a slit lamp biomicroscope and indirect ophthalmoscope to evaluate ocular surface morphology and anterior and posterior segment inflammation on all animals prior to injection to serve as a baseline for enrollment into the study as well as at days 2, 4, 8, 15, 22, and 29. Animals were not tranquilized for the examinations.

Digital planar photographs of the eye were obtained at days 2, 4, 8, 15, 22, and 29.

In addition, tonometry was performed by a veterinary ophthalmologist or a trained technician. Intraocular pressure (IOP) was measured in both eyes of all animals at days 2, 4, 8, 15, 22, and 29. Measurements were taken with a Tonovet tonometer. The IOP measurements were performed without the use of a topical anesthetic. The Tonovet tonometer probe was directed to gently contact the central cornea. Six consecutive measurements were obtained and the average IOP shown on the display was recorded. Three independent measurements were obtained and recorded for each eye.

Pachymetry was also performed by a veterinary ophthalmologist or trained technician on days 2, 4, 8, 15, 22, and 29. Corneal thickness was measured in both eyes using an ultrasonic pachymeter. Animals were given a topical anesthetic (0.5% proparacaine HCl per eye). The probe of the pachymeter was applied to the central cornea and the measured displayed was recorded.

Optical coherence tomography (OS only) was also measured in the animals. Initially, the animals were sedated immediately prior to the procedure with ketamine/xylazine cocktail (20-50/4-10 mg/kg IM) and one drop of 0.5% proparacaine HCl was applied OS. The OCT imaging procedures were performed by a trained technician. At baseline and on days 4, 8, 15, 22, and 28, animals underwent OCT imaging procedures of the anterior segment (anterior segment module) of the eye over time. If needed, a wire eyelid speculum was placed, and a central scan of the cornea was taken to measure central corneal thickness. The depth of lesions and overall corneal thickness of the left eye were measured using calipers in the instrument software.

Corneal specular microscopy for corneal endothelial cell (CEC) counts (OS only) was also measured in the animals. The animals were sedated immediately prior to the procedure with ketamine/xylazine cocktail (20-50/4-10 mg/kg IM) and one drop of 0.5% proparacaine HCl was applied OS. Imagine procedures were performed by a trained technician. At baseline and on days 4, 8, 15, 22, and 28, animals underwent imaging procedures of the cornea. A wire eyelid speculum was placed and a thin layer of eye lubricant was applied to the ocular surface. The camera lens was placed centrally to contact the cornea and was focused to visualize the endothelium. Central images were collected to analyze the morphology and density of the endothelial cells. Following imaging, animals were allowed to recover normally or were euthanized at the final timepoint.

Following euthanasia, eyes were enucleated and rinsed with fresh ice-cold PBS. Aqueous humor was removed, placed into a pre-weighted tube, reweighed, and snap frozen by immersion in liquid nitrogen. Care was taken to avoid collecting aqueous humor from the central cornea so as not to impede the subsequent IHC. Samples were stored at −80° C.

Following euthanasia, eyes designated for corneal flatmounts were immediately enucleated, corneas were dissected with a 2-3 mm scleral margin and then fixed in 4% PFA for 5 minutes. Tissues were permeabilized in acetone for 5 minutes at −20° C. Tissues were stored at 4° C. in PBS until processed for immunohistochemistry. Tissue was blocked in PBS containing 4% BSA for 1 hour. Primary antibody incubation (Ki67) occurred overnight at 4° C., then the corneas were washed 3×10 minutes in PBS and all other antibody incubations were in blocking buffer for 1 hour at room temperature (RT) followed by 3×5 minutes PBS washes. All antibody incubations and washes were performed on a nutator to gently agitate the samples. Once the corneas finished their final wash, curved scissors were used to remove nearly all of the sclera margin, leaving one small section to be handled with forceps. Using a #11 scalpel blade, 4-6 small incisions were made around the periphery to flatten the cornea. The cornea was carefully placed within a spacer window on a slide and 3-4 drops of mounting media (90% glycerol, 0.5% N-propyl gallate, 20 mM Tris pH 8) was applied using a 2 mL transfer pipette. The slide was then cover-slipped. Slides were imaged on an Olympus Bx63 upright fluorescent microscope using plan-apochromatic objectives (4×, 10×, 20×, and 40×).

Following euthanasia, eyes designated for cryosectioning and immunohistochemistry (IHC) were immediately enucleated, the superior surface marked with a tissue marker, and the corneas were dissected with a 4-5 mm scleral margin taking care to include the ciliary body. The iris and lens were not removed. All of the corneas were fixed in 4% PFA for 5 minutes. Following fixation, all of the eyes were cryoprotected through a gradient of sucrose (10%-30%), embedded in OCT medium, and frozen on dry ice. The corneas were embedded such that the temporal surface was sectioned through first (sagittal plane) with all sections containing both superior and inferior surfaces. Starting midway between the periphery and central cornea, two (n=2) slides containing serial sections were obtained, then 200-300 um were advanced with sections discarded and two additional slides collected such that the central cornea and immediate surrounding area was sectioned through and a maximum of 30 slides was collected. The slides were stained with the following primary antibodies diluted in blocking buffer: 1/100 mouse anti-Ki67 (Cell Signaling Tech) and 1/200 goat anti-smooth muscle actin (Novus). Following 3×5 minutes of PBS washes, the slides were incubated in 1/100 donkey anti-mouse biotin (Jackson Immuno) for 1 h at RT in blocking buffer. The slides were then washed 3×5 minutes in PBS and incubated with the following fluorphore-conjugated antibodies 1/200 Streptavidin Cy5 (Jackson Immuno), 1/200 donkey anti-goat Cy2 (Jackson Immuno), 1/100 mouse anti-NaK ATPase AlexaFluor 555 (EMD Millipore), and 1/1000 DAPI. The slides were imaged on an Olympus Bx63 fluorescent microscope using plan-apochromatic objectives (4×, 10×, 20×, and 40×).

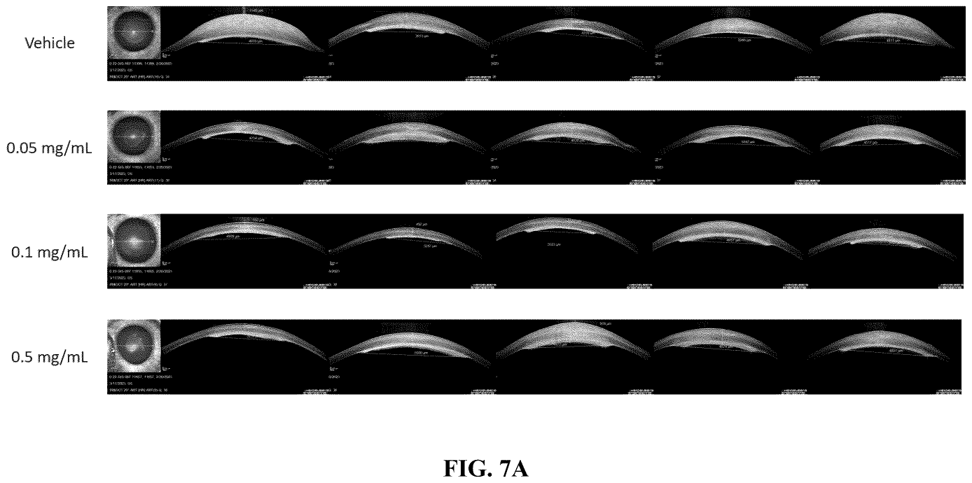

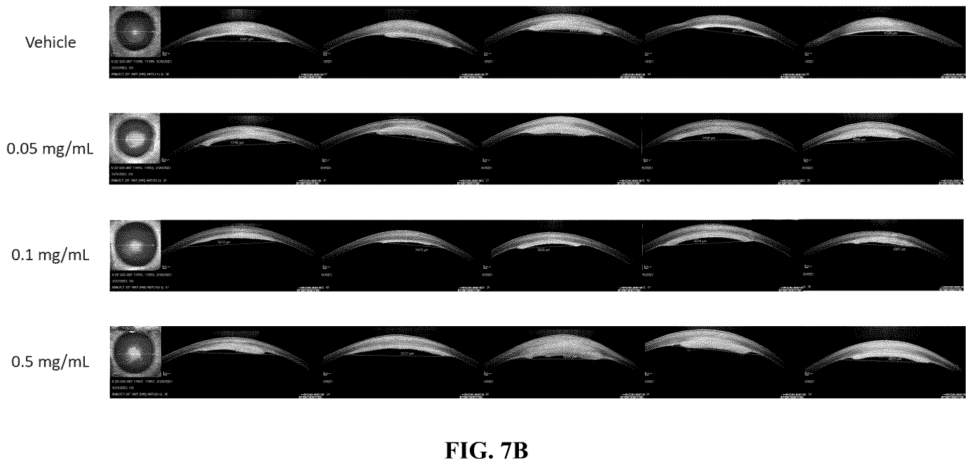

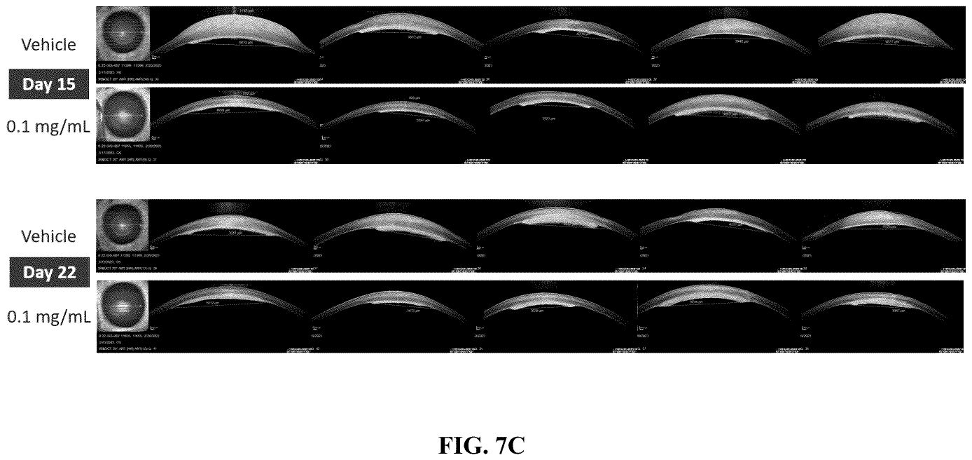

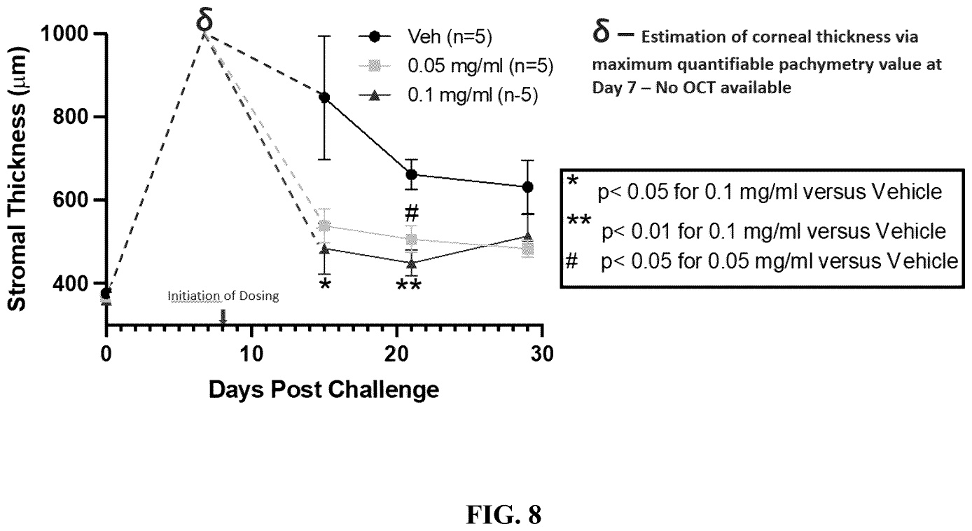

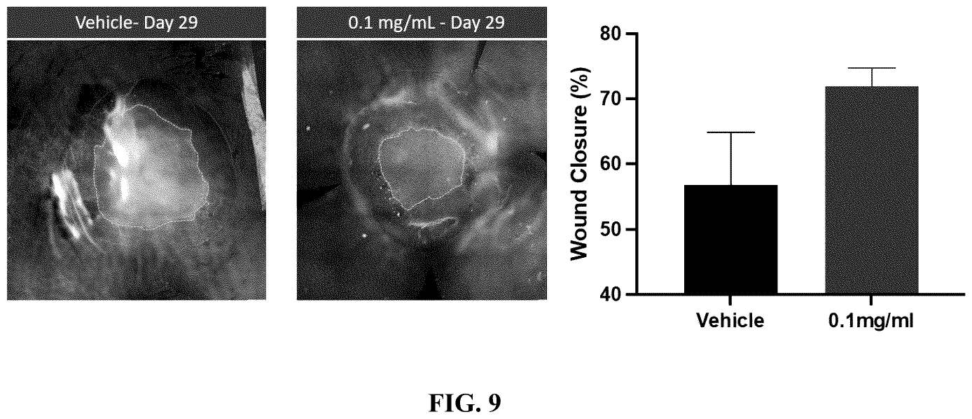

Optical coherence tomography (OCT) images were obtained at Day 15 (FIG. 7A) and Day 22 (FIG. 7B) and show changes to stromal thickness in untreated eyes compared to the eyes treated with 0.05 mg/mL, 0.1 mg/mL, and 0.5 mg/mL K173. A more specific comparison of 0.1 mg/mL K173 against vehicle at Day 15 and Day 22 is provided in FIG. 7C. The stromal thickness provides a measure of corneal endothelial function and edema. K173 was shown to decrease corneal edema as evidenced by the stromal thickness measurements. As shown in FIG. 8, statistically significant efficacy was seen soon after dosing occurred with the two lowest treatment options when assessing corneal stromal thickness by OCT. In addition, corneal flat mount analysis was performed on 3 animals per test group to assess the rate of wound closure and it was shown that the topical administration of K173 enhanced wound healing (FIG. 9).

### Example 9. In Vivo Topical Administration Study—Rat

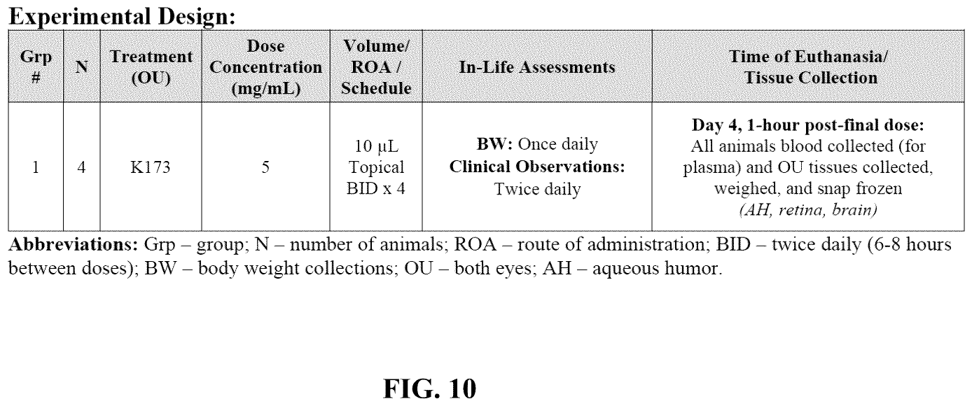

To evaluate the pharmacokinetics of the peptide formulations following topical administration a study in rats was undertaken. Animals were acclimated to the study environment for a minimum of 3 days and, upon completion of the acclimation period, each animal was physically examined for determination of suitability for study participation. Animals determined to be in good health were randomized into study groups (N=4). Animals will be uniquely identified by corresponding cage card number and ear tag. A Table summarizing the parameters regarding the study is provided in FIG. 10.

The K173 (KR-072; SEQ ID NO: 72) peptide was formulated in a 5 mg/ml sterile solution with HPMC, histidine buffer, and mannitol. The designated peptide formulations (10 L) were administered to the eye of each animal using a calibrated pipette. With the animal manually restrained, the upper eyelid of the eye was gently elevated to expose the cornea. Treatment was then be applied to the cornea without contacting the eye with the pipette tip. The animal was then allowed to blink several times while still manually restrained to distribute the applied solution over the eye prior to being returned to the cage. Treatment was administered twice daily (BID) with approximately 6-8 hours between BID doses.

All animals underwent twice daily cage side clinical observations with particular attention paid to the eyes. Animals were not tranquilized for the examinations.

When animals were terminal, blood was collected by inserting a 25G needle into the heart and the animal was exsanguinated and the blood was collected into K₂EDTA tubes for plasma collection. After collection, the tubes were gently mixed by inverting the tubes 5-8 times. The samples were centrifuged at approximately 4° C. for 10 minutes at 2000×g in a swinging bucket refrigerated centrifuge within 20 minutes of the blood collection. Once centrifuged, the clear plasma was transferred to a prelabelled polypropylene tube, snap frozen and stored at approximately −80° C.

Following euthanasia, eyes were enucleated and rinsed twice with fresh ice-cold PBS. Aqueous humor was removed via a 27- or 30-gauge syringe, placed into a pre-weighted polypropylene tube, reweighed, and snap frozen by immersion in liquid nitrogen. The anterior segment and lens were removed and the neurosensory retina was dissected into pre-weighed 2 mL polypropylene tube, re-weighed and snap frozen. The brain was collected and weighted and the hypothalamus, hippocampus, and remining brain were dissected, placed in pre-weighted polypropylene tubes, re-weighed, and snap frozen. All tissues were stored at −80° C.

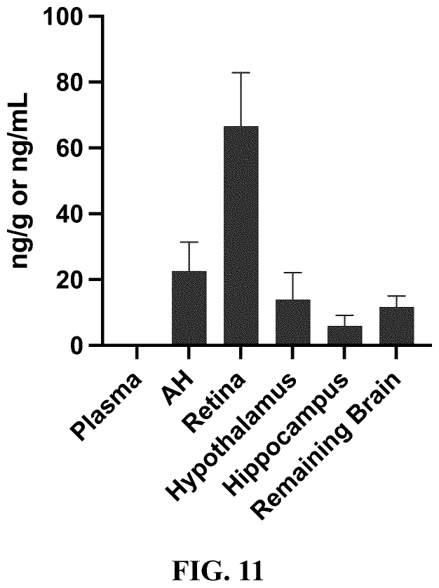

As shown in FIG. 11, K173 was shown to be delivered to the retina as well as to several areas of the brain. Following four days of BID topical dosing plasma levels were at or below BQL (0.1 ng/mL), high levels of K173 was found in the neural retina suggesting both corneal and scleral penetration, and K173 was found in all parts of the brains including the hypothalamus and the hippocampus. The concentrations of K173 in the aqueous humor, retina, hypothalamus, hippocampus, remaining brain tissue, and plasma was measured and the values are provided in Table 7.

TABLE 7
Concentration of K173 in Various Tissues in Rats

| | | K173 Concentrations in Mouse Sample | | | | | |
|---|---|---|---|---|---|---|---|
| | | Aqueous | | | | Remaining | |
| Group 1 | | Humor | Retina | Hypothalamus | Hippocampus | Brain | Plasma |
| Animal | | Concentration Units | | | | | |
| ID | OS/OD | ng/mL | ng/g | ng/g | ng/g | ng/g | ng/mL |
| 02 | OS | 16.2 | 101 | 937 | 0.778 | 14.8 | 0.135 |
| | OD | 55.1 | 40.3 | | | | |
| 03 | OS | BQL(2.00) | 15.9 | 7.61 | 0.535 | 4.62 | BQL |
| | OD | 0.442 | 29.3 | | | | (0.100) |
| 04 | OS | 25.9 | 21.1 | 4.06 | 13.3 | 19.3 | BQL |
| | OD | 450 | 76.6 | | | | (0.100) |
| 05 | OS | 0.524 | 125 | 30.2 | 9.14 | 8.21 | BQL |
| | OD | 37.5 | 124 | | | | (0.100) |

### Example 10. In Vivo Topical Administration Study—Minipig

To evaluate the effects of the peptide formulations following topical administration a study in minipigs was undertaken. Animals were acclimated to the study environment for a minimum of 1 week. At the completion of the acclimation period, each animal was physically examined for determination of suitability for study participation. Examinations included, but were not limited to, the skin and external ears, eyes, and abdomen, neurological behavior, and general body condition. Animals determined to be in good health were randomized into one of three study groups (N=12; 4 animals per study group) and uniquely identified by corresponding cage card number and ear tag.

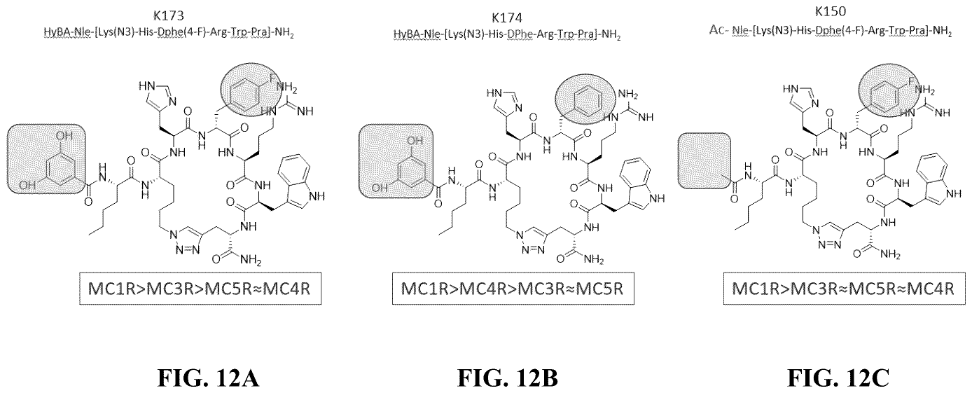

The K150 (KR-049; SEQ ID NO: 49), K173 (KR-072; SEQ ID NO: 72) and K174 (KR-073; SEQ ID NO: 73) peptides, the structures of which are depicted in FIGS. 12A, 12B and 12C, respectively, were each formulated in a 5 mg/mL sterile solution with HPMC, histidine buffer, and mannitol and stored at room temperature protected from light. The designated peptide formulations (35 L) were administered to the eye of each animal using a calibrated pipette. With the animal manually restrained, the upper eyelid of the eye was gently elevated to expose the cornea. Treatment was then be applied to the cornea without contacting the eye with the pipette tip. The animals were then allowed to blink several times while still manually restrained to distribute the applied solution over the eye prior to returning the animal to the cage. Treatment was administered twice daily (BID) with approximately 6-8 hours between each BID dose, with tissue collection one hour after the last dose. Tissues and blood collected include plasma, aqueous humor, iris/ciliary body, vitreous humor, eye cup containing the retinal pigment epithelium, choroid, sclera, cornea, retina, optic nerve, and brain.

Blood (3 mL) was collected IV or ICAR for terminal collections and placed into K₂EDTA tubes for plasma acquisition. After whole blood was added, the tubes were gently mixed by inverting the tubes 5-8 times. Blood samples were stored on wet ice for up to 30 minutes prior to plasma processing. The samples were centrifuged at 4° C. for 10 minutes at 2000 g in a swinging bucket refrigerated centrifuge. After centrifugation, the clear plasma was transferred to prelabelled polypropylene tubes, snap frozen, and stored frozen at approximately −80° C.

Following euthanasia, eyes were enucleated and rinsed twice with fresh ice-cold PBS. Aqueous humor was removed via a 27 or 30 G syringe, weighed in polypropylene screw cap tubes, and snap frozen by immersing in liquid nitrogen. Eyes were dissected while frozen. All tissues were placed into individual vials, weighed, and snap frozen. The brain was collected and weighed. The hypothalamus, hippocampus and remaining brain was then dissected and weighed. 100-200 mg of each section was placed in pre-weighed polypropylene tubes, reweighed, and snap frozen. The remaining tissue was discarded. All tissues were stored frozen at −80° C. on dry ice.

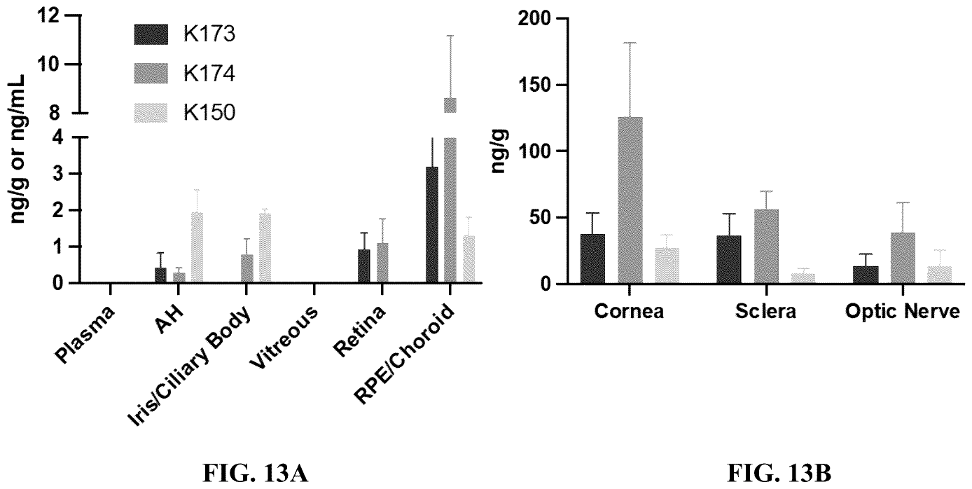

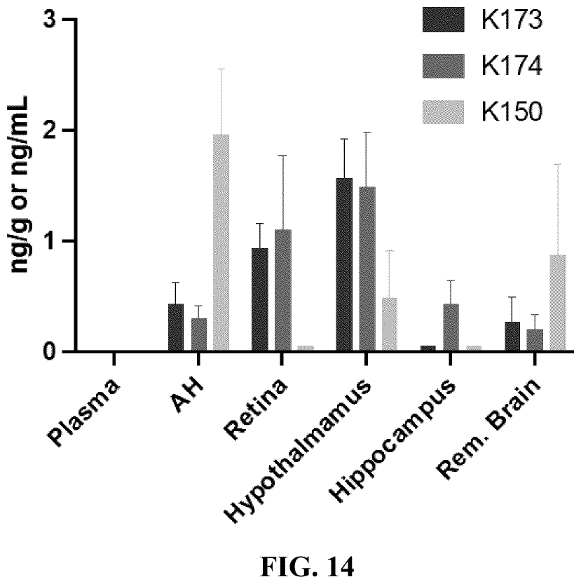

As shown in FIG. 13A and FIG. 13B, the K173 and K174 peptide formulations appeared to have a similar distribution pattern, while the K150 formulation demonstrated decreased scleral and retinal exposure and enhanced melanin binding. The observed distribution pattern was consistent with that observed in the rat study. As shown in FIG. 14, AH levels for all three peptides were lower than that observed in the rabbit and rat studies, and the K150 peptide demonstrated enhanced corneal permeability similar to that observed in the rabbit study. As also shown in FIG. 14, all three peptides were detected in the CNS of the animals, with no behavioral or metabolic changes noted in these animals despite the significant exposure. The foregoing results therefor confirm that the peptides disclosed herein can be delivered topically for retinal indications.

An ideal MCR agonist candidate will have 100× potency over native peptide to MC1R. The MCR agonists in vivo ocular permeability should allow for concentrations well exceeding EC₅₀ values. Assessment of MCR agonist candidates include K110, K150, K172, K173, and K174 (Table 8).

TABLE 8
EC₅₀ Values

| | | | | | | Topical |
|---|---|---|---|---|---|---|
| | | | | | | Ocular |
| Peptide | EC₅₀(nM)- | EC₅₀(nM)- | EC₅₀(nM)- | EC₅₀(nM)- | | Permeability |
| ID | MC1R | MC3R | MC4R | MC5R | Solubility | (nM ± SEM) |
| αMSH | 0.3 | 193.47 | 112.38 | 173.83 | <1 mg/mL | BQL |
| K110 | 0.011 | 44.10 | 45.71 | 140.4 | >5 mg/mL | 1.43 ± 1.01 |
| K150 | 0.019 | 21.3 | 16.11 | 25.19 | 9.95 mg/mL | 23.9 ± 10.8 |
| K172 | 0.0023 | 49.01 | 103.41 | 73.93 | 6.10 mg/mL | 2.5 ± 0.2 |
| K173 | 0.0019 | 43.47 | 98.57 | 78.05 | 6.47 mg/mL | 15.5 ± 2.6 |
| K174 | 0.0041 | 126.55 | 13.89 | 133.66 | 7.69 mg/mL | 12.3 ± 3.8 |

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A peptide of the formula: Z-XAA1-XAA2-XAA3-XAA4-XAA5-XAA6-XAA7-XAA8-XAA9-XAA10-XAA11-XAA12-XAA13-Y, wherein:
Z is Ac, PBA, octanoyl-PEG8G-G, PyAA, HyBA, HyPA, MoPA, HymBA, C7, C8, or C9;
AA1, AA2, AA3 are absent;
AA4 is Met or Nle or Lys(N3) or optionally Lys(N3) forms a cyclic peptide as a triazole group with Pra group at the 11 position;
AA5 is Glu, Lys(N3), D-Lys(N3), Dab(N3), Pra, or Orn(N3) or optionally Lys(N3), D-Lys(N3), Dab(N3), Pra, or Om(N3) form a cyclic peptide as a triazole group with Pra, Dab(N3), Hpra, Lys(N3) or D-Pra group at the 10 position;
AA6 is His, NMe-His, Tyr, Tyr(Me), or Phe(4-NH2);
AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me);
AA8 is Arg, NMe-Arg, Harg, or Arg(Me)2;
AA9 is Trp, NMe-Trp, or Trp(Me);
AA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Om(N3) group at the 10 position;
AA11 is absent, Pra or optionally Pra forms a cyclic peptide as a triazole group with Lys(N3) group at the 4 position;
AA12, AA13 are absent; and Y is NH2 or absent.

2. The peptide of claim 1, comprising an amino acid sequence selected from the group consisting of SEQ ID NO: 8, SEQ ID NO: 9, SEQ ID NO: 10, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, and SEQ ID NO: 75.

3. (canceled)

4. (canceled)

5. (canceled)

6. A peptide of the formula: Z-XAA1-XAA2-XAA3-XAA4-XAA5-XAA6-XAA7-XAA8-XAA9-XAA10-XAA11-XAA12-XAA13-Y, wherein:
(a) Z is absent, or comprises an N-terminus sequence;
(b) AA1 is absent, comprises a Ser¹ amino acid, or comprises a Ser¹ substitution moiety;
(c) AA2 is absent, comprises a Tyr² amino acid, or comprises a Tyr² substitution moiety;
(d) AA3 is absent, comprises a Ser³ amino acid, or comprises a Ser³ substitution moiety;
(e) AA4 is absent, comprises a Met⁴ amino acid, or comprises a Met⁴ substitution moiety;
(f) AA5 is absent, comprises a Glu⁵ amino acid, or comprises a Glu⁵ substitution moiety;
(g) AA6 is absent, comprises a His⁶ amino acid, or comprises a His⁶ substitution moiety;
(h) AA7 is absent, comprises a Phe⁷ amino acid, or comprises a Phe⁷ substitution moiety;
(i) AA8 is absent, comprises a Arg⁸ amino acid, or comprises a Arg⁸ substitution moiety;
(j) AA9 is absent, comprises a Trp⁹ amino acid, or comprises a Trp⁹ substitution moiety;
(k) AA10 is absent, comprises a Gly¹⁰ amino acid, or comprises a Gly¹⁰ substitution moiety;
(l) AA11 is absent, comprises a Lys¹¹ amino acid, or comprises a Lys¹¹ substitution moiety;
(m) AA12 is absent, comprises a Pro¹² amino acid, or comprises a Pro¹² substitution moiety;
(n) AA13 is absent, comprises a Val¹³ amino acid, or comprises a Val¹³ substitution moiety; and
(o) Y is absent, or comprises a C-terminus sequence.

7. The peptide of claim 6, wherein Z comprises an N-terminus sequence selected from: Ac, norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, (S)-2-amino-3-(1H-indazol-3-yl)propanoic acid, Ac-Nle; Ac-Arg; 7-Ahept; BzlSO₂, PyrPropHep; 2-Nac; Nba; Npa; Pba; Ppa; Tos; or a combination thereof; optionally wherein the N-terminus sequence comprises a string of 5 or 6 amino acids (G/L-G/L-G/L-G/L-G/L-G/L), each independently selected from Glu or Lys; or optionally wherein the N-terminus sequence comprises poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., PLy-PGa-α-MSH); and/or
wherein AA1 comprises a Ser¹ amino acid, or comprises a Ser¹ substitution moiety selected from: D-Ser, NMe-Ser, Ile, Thr, Tyr, Tyr(Me), or D-stereoisomers thereof; and/or
wherein AA2 comprises a Tyr² amino acid, or comprises a Tyr² substitution moiety selected from: D-Tyr, Ile, 2-Nal, 2-Pal, 3-Pal, Phe, 3Cl-Phe, 4F-Phe, 4C1-Phe, 4M-Phe, 4T-Phe, Phe(2,4-DiCl), Ser, Thr, Tic, Tyr(Me), or D-stereoisomers thereof; and/or
wherein AA3 comprises a Ser³ amino acid, or comprises a Ser³ substitution moiety selected from: D-Ser, Ile, Leu, Nle, Tyr, Val, BrAc, or D-stereoisomers thereof, and optionally forms a cyclic peptide with group at the 9, 10, 11 or 12 position; and/or
wherein AA4 comprises a Met⁴ amino acid, or comprises a Met⁴ substitution moiety selected from: Lys, Lys(N3), BrAc, R4-R10, S4-S10, D-Met, Asp, Can, Cba, Cha, Cpna, Cpra, Cys, D-Cys, hCys, D-hCys, Glu, Gly, Hcy, Hle, Ile, Leu, (cyclohexyl)Gly, Nle, Nle(Met), Pen, D-Pen, Ser, Tyr, or Val, or D-stereoisomers thereof and optionally forms a cyclic peptide with group at the 9, 10, 11 or 12 position; and/or
wherein AA5 comprises a Glu⁵ amino acid, or comprises a Glu⁵ substitution moiety selected from: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OmN3, Nle, Orn, Ser, Succ, Tyr, or 4-aminobutyric acid, or D-stereoisomers thereof and optionally forms a cyclic peptide with group at the 9, 10, 11 or 12 position; and/or
wherein AA6 comprises a His⁶ amino acid, or comprises a His⁶ substitution moiety selected from: D-His, NMe-His, NMe-D-His, Phe-4-NH2, Aib, Aic, Ala, D-Ala, Arg, Asn, Asp, Cha, Cha, Cit, Cys, D-Cys, Dab, Dap, Gln, Glu, His(1-Me), His(3-Me), Hyp, Hyp(Bzl), Ile, Leu, Lys, Met, Met(O), Met(O2), Nle, D-Nle, Om, 2-Pal, 3-Pal, 4-Pal, Phe, Pro, Sar, Ser, Ser(Bzl), Thr, Thr(OBzl), Tic, Tle, Trp, Tyr, Tyr(Me), or Val, cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, (3-furyl)alanine, a Phe moiety optionally substituted by halogen, hydroxyl, alkoxy, nitro, benzoyl, methyl, trifluoromethyl, amino, or cyano groups, or D-stereoisomers thereof and optionally forms a cyclic peptide with group at the 9, 10, 11 or 12 position; and/or
wherein AA7 comprises a Phe⁷ amino acid, or comprises a Phe⁷ substitution moiety selected from: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), D-Phe (2,4-diCl), Phe(2,4-diMe), D-Phe(2,4-diMe), Phe(3-Cl), D-Phe(3-Cl), Phe(3-CN), Phe(4-NH2), D-Phe(4-NH2), Phe(3,4-diF), D-Phe(3-CN), Phe(3-F), D-Phe(3-F), Phe(4-F), D-Phe(4-F), Phe(3-Me), D-Phe(3-Me), D-Phe (3,4-DiCl), Phe(4-CF3), D-Phe(4-CF3), Phe(4-Cl), D-Phe(4-Cl), Phe(4-CN), D-Phe(4-CN), Phe(4-F), D-Phe(4-F), Phe(4-Me), D-Phe(4-Me), Phe(4-OMe), D-Phe(4-OMe), Tic, Thi, D-Thi, Trp, Tyr, D-Tyr, Tyr(Me), Tyr(Me), D-Tyr(Me), Val, or D-Val, or D-stereoisomers thereof; and/or
wherein AA8 comprises a Arg⁸ amino acid, or comprises a Arg⁸ substitution moiety selected from: hArg, norArg, D-Arg, Arg(Me), Arg(Me)2, Ala, Cys, D-Cys, Dab, Dap, Dpr(beta-Ala), Leu, Lys, hLys, Nle, (Nlys)Gly, NMe-Arg, Om, Phe, Phe(4-Cl), D-Phe(4-Cl), Ser, or Trp, or D-stereoisomers thereof; and/or
wherein AA9 comprises a Trp⁹ amino acid, or comprises a Trp⁹ substitution moiety selected from: D-Trp, Trp(6-Me), Trp(7-Me), Aic, Atc, Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq, Tpi, or D-Tpi, R4-R10, S4-S10, Ndab, Orn or D-stereoisomers thereof and optionally forms a cyclic peptide with group at the 3, 4 or 5 position; and/or
wherein AA10 comprises a Gly¹⁰ amino acid, or comprises a Gly¹⁰ substitution moiety selected from: D-Gly, Ala, D-Ala, Arg, 6-Ahx, Cys, D-Cys, Daa, Dab, DabN3, Glu, Lys, D-Lys, hLys, LysN3, Orn, Pra, Hpra, Trp, R4-R10, S4-S10, or 5-aminopentanoic acid, or D-stereoisomers thereof and optionally forms a cyclic peptide with group at the 3, 4 or 5 position; and/or
wherein AA1l comprises a Lys¹¹ amino acid, or comprises a Lys¹¹ substitution moiety selected from: D-Lys, Ala, Asn, Asp, Cys, D-Cys, Dab, Glu, hGlu, Hgin, Gly, Lys(Me)2, Pra, or Orn, or D-stereoisomers thereof and optionally forms a cyclic peptide with group at the 3, 4 or 5 position; and/or
wherein AA12 comprises a Pro¹² amino acid, or comprises a Pro¹² substitution moiety selected from: D-Pro, Ala, Asp, Cys, Gly, Ile, Leu, Met, Phe, Ser, Trp, Val, or D-stereoisomers thereof and optionally forms a cyclic peptide with group at the 3, 4 or 5 position; and/or
wherein AA13 comprises a Val¹³ amino acid, or comprises a Val¹³ substitution moiety selected from: D-Val, NMe-Val, Ala, Can, Cba, Cha, Cpna, Cpra, Cys, Hcy, Hle, Ile, Leu, Met, Nle, Pro, or D-stereoisomers thereof; and/or
wherein Y comprises a C-terminus sequence selected from: NH₂, OH, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, or Trp-NH₂.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The peptide of claim 6, wherein Z is Ac, PBA, octanoyl-PEG8G-G, PyAA, HyBA, HyPA, MoPA, HymBA, C7, C8, or C9; AA1 is Ser or absent; AA2 is Tyr or absent; AA3 is Met or absent; AA4 is Met or Nle or absent; AA5 is Glu; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Trp, or Ala; AA11 is Lys, Gly, Asn, HgIn, Lys(Me)2, or absent; AA12 is Pro or absent; AA13 is Val or absent; and Y is NH2 or absent.

23. The peptide of claim 22, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 35.

24. The peptide of claim 6, wherein Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Glu, Lys(N3), D-Lys(N3), Dab(N3), Pra, or Orn(N3) or optionally Lys(N3), D-Lys(N3), Dab(N3), Pra, or Om(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; AA11, AA12, AA13 are absent; and Y is NH2 or absent.

25. The peptide of claim 24, comprising an amino acid sequence selected from the group consisting of SEQ ID NO: 8, SEQ ID NO: 9, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, and SEQ ID NO: 75.

26. The peptide of claim 6, wherein Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Glu, Lys(N3), D-Lys(N3), Dab(N3), Pra, or Om(N3) or optionally Lys(N3), D-Lys(N3), Dab(N3), Pra, or Om(N3) form a cyclic peptide as a triazole group with Pra, Dab(N3), Hpra, Lys(N3) or D-Pra group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; AA11 is Lys, Gly, Asn, HgIn, or Lys(Me)2; AA12, AA13 are absent; and Y is NH2 or absent.

27. The peptide of claim 26, comprising an amino acid sequence selected from the group consisting of SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71.

28. The peptide of claim 6, wherein Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; AA11, AA12, AA13 are absent; and Y is NH2 or absent.

29. The peptide of claim 28, comprising an amino acid sequence selected from the group consisting of SEQ ID NO: 38 and SEQ ID NO: 39.

30. (canceled)

31. The peptide of claim 6, wherein Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Cys or α-Me-Cys or optionally Cys or α-Me-Cys form a cyclic peptide with Cys or α-Me-Cys group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Cys or α-Me-Cys or optionally Cys or α-Me-Cys form a cyclic peptide with Cys or α-Me-Cys group at the 5 position; AA11 is absent, Lys, Gly, Asn, HgIn, or Lys(Me)2; AA12, AA13 are absent; and Y is NH2 or absent.

32. The peptide of claim 31, comprising an amino acid sequence of SEQ ID NO: 40.

33. The peptide of claim 6, wherein Z is Ac, PBA, octanoyl-PEG8G-G, PyAA, HyBA, HyPA, MoPA, HymBA, C7, C8, or C9; AA1, AA2, AA3 are absent; AA4 is Met or Nle; AA5 is Dab or optionally Dab forms a cyclic peptide with Dab group at the 10 position; AA6 is His, NMe-His, Tyr, Tyr(Me), or Phe(4-NH2); AA7 is Phe, D-Phe, NMe-Ph, D-NMe-Ph, Phe(4-CF3), Phe(4-F), D-Phe(4-CF3), D-Phe(4-F),Tyr, Tyr(Me), D-Tyr, or D-Tyr(Me); AA8 is Arg, NMe-Arg, Harg, or Arg(Me)2; AA9 is Trp, NMe-Trp, or Trp(Me); AA10 is Dab or optionally Dab forms a cyclic peptide with Dab group at the 5 position; AA11 is absent, Lys, Gly, Asn, HgIn, or Lys(Me)2; AA12, AA13 are absent; and Y is NH2 or absent.

34. The peptide of claim 33, comprising an amino acid sequence of SEQ ID NO: 41.

35. (canceled)

36. A peptide comprising an amino acid sequence selected from the group consisting of SEQ ID NO: 1, SEQ ID NO: 2, SEQ ID NO: 3, and SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, and SEQ ID NO: 75.

37. An α-MSH analog comprising a peptide of claim 1.

38. An α-MSH analog comprising a peptide portion conjugated to a non-peptide portion, wherein the peptide portion comprises a peptide of claim 1.

39. (canceled)

40. (canceled)

41. (canceled)

42. (canceled)

43. (canceled)

44. A pharmaceutical composition comprising an α-MSH analog of claim 37, and at least one pharmaceutically acceptable excipient.

45. A method for treating or preventing one or more ophthalmic indications in a subject, the method comprising administering to the subject a therapeutically effective amount of the pharmaceutical composition of claim 44.

46. (canceled)

47. (canceled)

48. (canceled)

49. (canceled)
